SEQUENCE LISTING (299 sequences)

SEQ ID NO: 1 - protein, 98 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 2 - protein, 11 aa, synthetic construct
SGGGGSGGGGS

SEQ ID NO: 3 - protein, 13 aa, synthetic construct
SGGGGSGGGGSAP

SEQ ID NO: 4 - protein, 5 aa, synthetic construct
NFSQP

SEQ ID NO: 5 - protein, 5 aa, synthetic construct
KRTVA

SEQ ID NO: 6 - protein, 15 aa, synthetic construct
GGGGSGGGGSGGGGS

SEQ ID NO: 7 - protein, 107 aa, synthetic construct [sequence omitted]

SEQ ID NO: 8 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 9 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 11 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 12 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 13 - protein, 18 aa, synthetic construct
RTVAAPSVFIFPPSDEQL

SEQ ID NO: 14 - protein, 19 aa, synthetic construct
GQPKANPTVTLFPPSSEEL

SEQ ID NO: 15 - protein, 19 aa, synthetic construct
GQPKAAPSVTLFPPSSEEL

SEQ ID NO: 16 - protein, 12 aa, synthetic construct
WTFGQGTKVEIK

SEQ ID NO: 17 - protein, 12 aa, synthetic construct
YTFGQGTKLEIK

SEQ ID NO: 18 - protein, 12 aa, synthetic construct
FTFGPGTKVDIK

SEQ ID NO: 19 - protein, 12 aa, synthetic construct
LTFGGGTKVEIK

SEQ ID NO: 20 - protein, 12 aa, synthetic construct
ITFGQGTRLEIK

SEQ ID NO: 21 - protein, 11 aa, synthetic construct
TFGQGTKVEIK

SEQ ID NO: 22 - protein, 11 aa, synthetic construct
TFGQGTKLEIK

SEQ ID NO: 23 - protein, 11 aa, synthetic construct
TFGPGTKVDIK

SEQ ID NO: 24 - protein, 11 aa, synthetic construct
TFGGGTKVEIK

SEQ ID NO: 25 - protein, 11 aa, synthetic construct
TFGQGTRLEIK

SEQ ID NO: 26 - protein, 12 aa, synthetic construct
YVFGTGTKVTVL

SEQ ID NO: 27 - protein, 12 aa, synthetic construct
VVFGGGTKLTVL

SEQ ID NO: 28 - protein, 12 aa, synthetic construct
FVFGGGTQLIIL

SEQ ID NO: 29 - protein, 12 aa, synthetic construct
WVFGEGTELTVL

SEQ ID NO: 30 - protein, 12 aa, synthetic construct
AVFGGGTQLTVL

SEQ ID NO: 31 - protein, 11 aa, synthetic construct
VFGTGTKVTVL

SEQ ID NO: 32 - protein, 11 aa, synthetic construct
VFGTGTKVTVL

SEQ ID NO: 33 - protein, 11 aa, synthetic construct
VFGGGTQLIIL

SEQ ID NO: 34 - protein, 11 aa, synthetic construct
VFGEGTELTVL

SEQ ID NO: 35 - protein, 11 aa, synthetic construct
VFGGGTQLTVL

SEQ ID NO: 36 - protein, 29 aa, synthetic construct
TFGQGTKVEIKRTVAAPSVFIFPPSDEQL

SEQ ID NO: 37 - protein, 29 aa, synthetic construct
TFGQGTKLEIKRTVAAPSVFIFPPSDEQL

SEQ ID NO: 38 - protein, 29 aa, synthetic construct
TFGPGTKVDIKRTVAAPSVFIFPPSDEQL

SEQ ID NO: 39 - protein, 29 aa, synthetic construct
TFGGGTKVEIKRTVAAPSVFIFPPSDEQL

SEQ ID NO: 40 - protein, 29 aa, synthetic construct
TFGQGTRLEIKRTVAAPSVFIFPPSDEQL

SEQ ID NO: 41 - protein, 30 aa, synthetic construct
VFGTGTKVTVLGQPKANPTVTLFPPSSEEL

SEQ ID NO: 42 - protein, 30 aa, synthetic construct
VFGTGTKVTVLGQPKAAPSVTLFPPSSEEL

SEQ ID NO: 43 - protein, 30 aa, synthetic construct
VFGGGTKLTVLGQPKANPTVTLFPPSSEEL

SEQ ID NO: 44 - protein, 30 aa, synthetic construct
VFGGGTKLTVLGQPKAAPSVTLFPPSSEEL

SEQ ID NO: 45 - protein, 30 aa, synthetic construct
VFGGGTQLIILGQPKANPTVTLFPPSSEEL

SEQ ID NO: 46 - protein, 30 aa, synthetic construct
VFGGGTQLIILGQPKAAPSVTLFPPSSEEL

SEQ ID NO: 47 - protein, 30 aa, synthetic construct
VFGEGTELTVLGQPKANPTVTLFPPSSEEL

SEQ ID NO: 48 - protein, 30 aa, synthetic construct
VFGEGTELTVLGQPKAAPSVTLFPPSSEEL

SEQ ID NO: 49 - protein, 30 aa, synthetic construct
VFGGGTQLTVLGQPKANPTVTLFPPSSEEL

SEQ ID NO: 50 - protein, 30 aa, synthetic construct
VFGGGTQLTVLGQPKAAPSVTLFPPSSEEL

SEQ ID NO: 51 - protein, 29 aa, synthetic construct
  VARIANT 18: note = wherein X is any amino acid but preferably T or S, and it is preferably replaced by A
TFGQGTKVEIKRTVAAPXVFIFPPSDEQL

SEQ ID NO: 52 - protein, 29 aa, synthetic construct
  VARIANT 18: note = wherein X is any amino acid but preferably T or S, and it is preferably replaced by A
TFGQGTKLEIKRTVAAPXVFIFPPSDEQL

SEQ ID NO: 53 - protein, 29 aa, synthetic construct
  VARIANT 18: note = wherein X is any amino acid but preferably T or S, and it is preferably replaced by A
TFGPGTKVDIKRTVAAPXVFIFPPSDEQL

SEQ ID NO: 54 - protein, 29 aa, synthetic construct
  VARIANT 18: note = wherein X is any amino acid but preferably T or S, and it is preferably replaced by A
TFGGGTKVEIKRTVAAPXVFIFPPSDEQL

SEQ ID NO: 55 - protein, 29 aa, synthetic construct
  VARIANT 18: note = wherein X is any amino acid but preferably T or S, and it is preferably replaced by A
TFGQGTRLEIKRTVAAPXVFIFPPSDEQL

SEQ ID NO: 56 - protein, 11 aa, synthetic construct
  VARIANT 10: note = wherein X is any amino acid but preferably T or S, and it is preferably replaced by A
EIKRTVAAPXV

SEQ ID NO: 57 - protein, 11 aa, synthetic construct
  VARIANT 10: note = wherein X is any amino acid but preferably T or S, and it is preferably replaced by A
DIKRTVAAPXV

SEQ ID NO: 58 - protein, 8 aa, synthetic construct
  VARIANT 7: note = wherein X is any amino acid but preferably T or S, and it is preferably replaced by A
RTVAAPXV

SEQ ID NO: 59 - skipped

SEQ ID NO: 60 - protein, 30 aa, synthetic construct
  VARIANT 19: note = wherein X is any amino acid but preferably T or S, and it is preferably replaced by A
VFGTGTKVTVLGQPKANPXVTLFPPSSEEL

SEQ ID NO: 61 - protein, 30 aa, synthetic construct
  VARIANT 19: note = wherein X is any amino acid but preferably T or S, and it is preferably replaced by A
VFGTGTKVTVLGQPKAAPXVTLFPPSSEEL

SEQ ID NO: 62 - protein, 30 aa, synthetic construct
  VARIANT 19: note = wherein X is any amino acid but preferably T or S, and it is preferably replaced by A
VFGGGTKLTVLGQPKANPXVTLFPPSSEEL

SEQ ID NO: 63 - protein, 30 aa, synthetic construct
  VARIANT 19: note = wherein X is any amino acid but preferably T or S, and it is preferably replaced by A
VFGGGTKLTVLGQPKAAPXVTLFPPSSEEL

SEQ ID NO: 64 - protein, 30 aa, synthetic construct
  VARIANT 19: note = wherein X is any amino acid but preferably T or S, and it is preferably replaced by A
VFGGGTQLIILGQPKANPXVTLFPPSSEEL

SEQ ID NO: 65 - protein, 30 aa, synthetic construct
  VARIANT 19: note = wherein X is any amino acid but preferably T or S, and it is preferably replaced by A
VFGGGTQLIILGQPKAAPXVTLFPPSSEEL

SEQ ID NO: 66 - protein, 30 aa, synthetic construct
  VARIANT 19: note = wherein X is any amino acid but preferably T or S, and it is preferably replaced by A
VFGEGTELTVLGQPKANPXVTLFPPSSEEL

SEQ ID NO: 67 - protein, 30 aa, synthetic construct
  VARIANT 19: note = wherein X is any amino acid but preferably T or S, and it is preferably replaced by A
VFGEGTELTVLGQPKAAPXVTLFPPSSEEL

SEQ ID NO: 68 - protein, 30 aa, synthetic construct
  VARIANT 19: note = wherein X is any amino acid but preferably T or S, and it is preferably replaced by A
VFGGGTQLTVLGQPKANPXVTLFPPSSEEL

SEQ ID NO: 69 - protein, 30 aa, synthetic construct
  VARIANT 19: note = wherein X is any amino acid but preferably T or S, and it is preferably replaced by A
VFGGGTQLTVLGQPKAAPXVTLFPPSSEEL

SEQ ID NO: 70 - protein, 11 aa, synthetic construct
  VARIANT 10: note = wherein X is any amino acid but preferably T or S, and it is preferably replaced by A
VLGQPKANPXV

SEQ ID NO: 71 - protein, 11 aa, synthetic construct
  VARIANT 10: note = wherein X is any amino acid but preferably T or S, and it is preferably replaced by A
VLGQPKAAPXV

SEQ ID NO: 72 - protein, 11 aa, synthetic construct
  VARIANT 10: note = wherein X is any amino acid but preferably T or S, and it is preferably replaced by A
ILGQPKANPXV

SEQ ID NO: 73 - protein, 11 aa, synthetic construct
  VARIANT 10: note = wherein X is any amino acid but preferably T or S, and it is preferably replaced by A
ILGQPKAAPXV

SEQ ID NO: 74 - protein, 9 aa, synthetic construct
  VARIANT 8: note = wherein X is any amino acid but preferably T or S, and it is preferably replaced by A
GQPKANPXV

SEQ ID NO: 75 - protein, 9 aa, synthetic construct
  VARIANT 5: note = wherein X is any amino acid but preferably T or S, and it is preferably replaced by A
VAAPXVFIF

SEQ ID NO: 76 - protein, 31 aa, Homo sapiens
AVLQSSGLYSLSSVVTVPSSSLGTQTYICNV

SEQ ID NO: 77 - protein, 31 aa, Homo sapiens
AVLQSSGLYSLSSVVTVPSSNFGTQTYTCNV

SEQ ID NO: 78 - protein, 31 aa, Homo sapiens
AVLQSSGLYSLSSVVTVPSSSLGTQTYTCNV

SEQ ID NO: 79 - protein, 31 aa, Homo sapiens
AVLQSSGLYSLSSVVTVPSSSLGTKTYTCNV

SEQ ID NO: 80 - protein, 31 aa, synthetic construct
  VARIANT 16: note = wherein X is any amino acid but preferably T, and it is preferably replaced by E or D
AVLQSSGLYSLSSVVXVPSSSLGTQTYICNV

SEQ ID NO: 81 - protein, 31 aa, synthetic construct
  VARIANT 16: note = wherein X is any amino acid but preferably T, and it is preferably replaced by E or D
AVLQSSGLYSLSSVVXVPSSNFGTQTYTCNV

SEQ ID NO: 82 - protein, 31 aa, synthetic construct
  VARIANT 16: note = wherein X is any amino acid but preferably T, and it is preferably replaced by E or D
  VARIANT 16: note = wherein X is any amino acid but preferably T, and it is preferably replaced by E or D
AVLQSSGLYSLSSVVXVPSSSLGTQTYTCNV

SEQ ID NO: 83 - protein, 31 aa, synthetic construct
  VARIANT 16: note = wherein X is any amino acid but preferably T, and it is preferably replaced by E or D
AVLQSSGLYSLSSVVXVPSSSLGTKTYTCNV

SEQ ID NO: 84 - protein, 12 aa, synthetic construct
  VARIANT 10: note = wherein X is any amino acid but preferably T, and it is preferably replaced by E or D
GLYSLSSVVXVP

SEQ ID NO: 85 - skipped

SEQ ID NO: 86 - protein, 9 aa, synthetic construct
  VARIANT 5: note = X is any amino acid but preferably T, and it is preferably replaced by E or D and the primary mutation at position 187 in the CH1 domain is at position X
SSVVXVPSS

SEQ ID NO: 87 - protein, 9 aa, synthetic construct
  VARIANT 1: note = wherein X is any amino acid
  VARIANT 4: note = wherein X is any amino acid
  VARIANT 6: note = wherein X is any amino acid
XKRXVXAPA

SEQ ID NO: 88 - protein, 9 aa, synthetic construct
  VARIANT 4: note = wherein X is any amino acid
  VARIANT 6: note = wherein X is any amino acid
IKRXVXAPA

SEQ ID NO: 89 - protein, 9 aa, synthetic construct
  VARIANT 4: note = wherein X is any amino acid
  VARIANT 6: note = wherein X is any amino acid
LGQXKXNPA

SEQ ID NO: 90 - protein, 9 aa, synthetic construct
  VARIANT 4: note = wherein X is any amino acid
  VARIANT 6: note = wherein X is any amino acid
LGQXKXAPA

SEQ ID NO: 91 - protein, 9 aa, synthetic construct
  VARIANT 1: note = wherein X is any amino acid
  VARIANT 4: note = wherein X is any amino acid
  VARIANT 6: note = wherein X is any amino acid
  VARIANT 9: note = wherein X is any amino acid
XSLXSXVEX

SEQ ID NO: 92 - protein, 9 aa, synthetic construct
  VARIANT 1: note = wherein X is any amino acid
  VARIANT 4: note = wherein X is any amino acid
  VARIANT 6: note = wherein X is any amino acid
  VARIANT 9: note = wherein X is any amino acid
XSLXSXVDX

SEQ ID NO: 93 - protein, 9 aa, synthetic construct
YSLSSVVEV

SEQ ID NO: 94 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTKVXIKRXVXAPAVFIFPPSDEQL

SEQ ID NO: 95 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTKLXIKRXVXAPAVFIFPPSDEQL

SEQ ID NO: 96 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGPGTKVXIKRXVXAPAVFIFPPSDEQL

SEQ ID NO: 97 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGGGTKVXIKRXVXAPAVFIFPPSDEQL

SEQ ID NO: 98 - protein, 29 aa, synthetic construct
  VARIANT 9: note = wherein X is any amino acid but preferably A, and it is preferably replaced by R or T or W or V
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTRLXIKRXVXAPAVFIFPPSDEQL

SEQ ID NO: 99 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTKVXIKRXVXAPSVFIFPPSDEQL

SEQ ID NO: 100 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTKLXIKRXVXAPSVFIFPPSDEQL

SEQ ID NO: 101 - protein, 29 aa, synthetic construct
  VARIANT 9: note = Wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = is any amino acid but preferably preferably A, and it is preferably replaced by R or T or W or V
TFGPGTKVXIKRXVXAPSVFIFPPSDEQL

SEQ ID NO: 102 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGGGTKVXIKRXVXAPSVFIFPPSDEQL

SEQ ID NO: 103 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTRLXIKRXVXAPSVFIFPPSDEQL

SEQ ID NO: 104 - protein, 16 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTKVXIKRXVXA

SEQ ID NO: 105 - protein, 16 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTKLXIKRXVXA

SEQ ID NO: 106 - protein, 16 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGPGTKVXIKRXVXA

SEQ ID NO: 107 - protein, 16 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGGGTKVXIKRXVXA

SEQ ID NO: 108 - protein, 16 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGQGTRLXIKRXVXA

SEQ ID NO: 109 - protein, 12 aa, synthetic construct
  VARIANT 2: note = wherein X is any amino acid but preferably I, and it is preferably replaced by A or G or H or N or P or Q or S or T
  VARIANT 6: note = wherein X is any amino acid but preferably T, and it is preferably replaced by P
  VARIANT 8: note = wherein X is any amino acid but preferably A, and it is preferably replaced by R or T or W or V
VXIKRXVXAPAV

SEQ ID NO: 110 - protein, 12 aa, synthetic construct
  VARIANT 2: note = wherein X is any amino acid but preferably I, and it is preferably replaced by A or G or H or N or P or Q or S or T
  VARIANT 6: note = wherein X is any amino acid but preferably T, and it is preferably replaced by P
  VARIANT 8: note = wherein X is any amino acid but preferably A, and it is preferably replaced by R or T or W or V
LXIKRXVXAPAV

SEQ ID NO: 111 - protein, 9 aa, synthetic construct
  VARIANT 2: note = wherein X is any amino acid but preferably I, and it is preferably replaced by A or G or H or N or P or Q or S or T
  VARIANT 6: note = wherein X is any amino acid but preferably T, and it is preferably replaced by P
  VARIANT 8: note = wherein X is any amino acid but preferably A, and it is preferably replaced by R or T or W or V
VXIKRXVXA

SEQ ID NO: 112 - protein, 9 aa, synthetic construct
  VARIANT 2: note = wherein X is any amino acid but preferably I, and it is preferably replaced by A or G or H or N or P or Q or S or T
  VARIANT 6: note = wherein X is any amino acid but preferably T, and it is preferably replaced by P
  VARIANT 8: note = wherein X is any amino acid but preferably A, and it is preferably replaced by R or T or W or V
LXIKRXVXA

SEQ ID NO: 113 - protein, 8 aa, synthetic construct
  VARIANT 2: note = wherein X is any amino acid but preferably T, and it is preferably replaced by P
  VARIANT 4: note = wherein X is any amino acid but preferably A, and it is preferably replaced by R or T or W or V
RXVXAPAV

SEQ ID NO: 114 - skipped

SEQ ID NO: 115 - protein, 6 aa, synthetic construct
  VARIANT 2: note = wherein X is any amino acid but preferably A, and it is preferably replaced by R or T or W or V
VXAPAV

SEQ ID NO: 116 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGTGTKVTVLGQXKXNPAVTLFPPSSEEL

SEQ ID NO: 117 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGTGTKVTVLGQXKXAPAVTLFPPSSEEL

SEQ ID NO: 118 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTKLTVLGQXKXNPAVTLFPPSSEEL

SEQ ID NO: 119 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTKLTVLGQXKXAPAVTLFPPSSEEL

SEQ ID NO: 120 - protein, 30 aa, synthetic construct
  VARIANT 14: note = wherein X is any amino acid but preferably T, and it is preferably replaced by P
  VARIANT 16: note = wherein X is any amino acid but preferably T, and it is preferably replaced by P
VFGGGTQLIILGQXKXNPAVTLFPPSSEEL

SEQ ID NO: 121 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLIILGQXKXAPAVTLFPPSSEEL

SEQ ID NO: 122 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGEGTELTVLGQXKXNPAVTLFPPSSEEL

SEQ ID NO: 123 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGEGTELTVLGQXKXAPAVTLFPPSSEEL

SEQ ID NO: 124 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLTVLGQXKXNPAVTLFPPSSEEL

SEQ ID NO: 125 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLTVLGQXKXAPAVTLFPPSSEEL

SEQ ID NO: 126 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGTGTKVTVLGQXKXNPTVTLFPPSSEEL

SEQ ID NO: 127 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGTGTKVTVLGQXKXAPSVTLFPPSSEEL

SEQ ID NO: 128 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTKLTVLGQXKXNPTVTLFPPSSEEL

SEQ ID NO: 129 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTKLTVLGQXKXAPSVTLFPPSSEEL

SEQ ID NO: 130 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLIILGQXKXNPTVTLFPPSSEEL

SEQ ID NO: 131 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLIILGQXKXAPSVTLFPPSSEEL

SEQ ID NO: 132 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGEGTELTVLGQXKXNPTVTLFPPSSEEL

SEQ ID NO: 133 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGEGTELTVLGQXKXAPSVTLFPPSSEEL

SEQ ID NO: 134 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLTVLGQXKXNPTVTLFPPSSEEL

SEQ ID NO: 135 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLTVLGQXKXAPSVTLFPPSSEEL

SEQ ID NO: 136 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGTGTKVTVLGQXKXN

SEQ ID NO: 137 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGTGTKVTVLGQXKXA

SEQ ID NO: 138 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTKLTVLGQXKXN

SEQ ID NO: 139 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTKLTVLGQXKXA

SEQ ID NO: 140 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLIILGQXKXN

SEQ ID NO: 141 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLIILGQXKXA

SEQ ID NO: 142 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGEGTELTVLGQXKXN

SEQ ID NO: 143 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGEGTELTVLGQXKXA

SEQ ID NO: 144 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLTVLGQXKXN

SEQ ID NO: 145 - protein, 17 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
VFGGGTQLTVLGQXKXA

SEQ ID NO: 146 - protein, 9 aa, synthetic construct
  VARIANT 3: note = wherein X is any amino acid but preferably T, and it is preferably replaced by P
  VARIANT 5: note = wherein X is any amino acid but preferably A, and it is preferably replaced by R or T or W or V
GQXKXNPAV

SEQ ID NO: 147 - protein, 9 aa, synthetic construct
  VARIANT 3: note = wherein X is any amino acid but preferably T, and it is preferably replaced by P
  VARIANT 5: note = wherein X is any amino acid but preferably A, and it is preferably replaced by R or T or W or V
GQXKXAPAV

SEQ ID NO: 148 - protein, 6 aa, synthetic construct
  VARIANT 3: note = wherein X is any amino acid but preferably T, and it is preferably replaced by P
  VARIANT 5: note = wherein X is any amino acid but preferably A, and it is preferably replaced by R or T or W or V
GQXKXN

SEQ ID NO: 149 - protein, 6 aa, synthetic construct
  VARIANT 3: note = wherein X is any amino acid but preferably T, and it is preferably replaced by P
  VARIANT 5: note = wherein X is any amino acid but preferably A, and it is preferably replaced by R or T or W or V
GQXKXA

SEQ ID NO: 150 - protein, 6 aa, synthetic construct
  VARIANT 2: note = wherein X is any amino acid but preferably A, and it is preferably replaced by R or T or W or V
KXNPAV

SEQ ID NO: 151 - protein, 6 aa, synthetic construct
  VARIANT 2: note = wherein X is any amino acid but preferably A, and it is preferably replaced by R or T or W or V
KXAPAV

SEQ ID NO: 152 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein is any amino acid but preferably V, and it is preferably replaced by G
AVLQSSGLXSLXSVVTXPSSSLGTQTYICNV

SEQ ID NO: 153 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVTXPSSNFGTQTYTCNV

SEQ ID NO: 154 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVTXPSSSLGTQTYTCNV

SEQ ID NO: 155 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVTXPSSSLGTKTYTCNV

SEQ ID NO: 156 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVEXPSSSLGTQTYICNV

SEQ ID NO: 157 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVEXPSSNFGTQTYTCNV

SEQ ID NO: 158 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVEXPSSSLGTQTYTCNV

SEQ ID NO: 159 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVEXPSSSLGTKTYTCNV

SEQ ID NO: 160 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVDXPSSSLGTQTYICNV

SEQ ID NO: 161 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVDXPSSNFGTQTYTCNV

SEQ ID NO: 162 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVDXPSSSLGTQTYTCNV

SEQ ID NO: 163 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 17: note = wherein X is any amino acid but preferably V, and it is preferably replaced by G
AVLQSSGLXSLXSVVDXPSSSLGTKTYTCNV

SEQ ID NO: 164 - protein, 11 aa, synthetic construct
  VARIANT 2: note = wherein X is any amino acid but preferably Y, and it is preferably replaced by A or G or I or N or S or T or V or W
  VARIANT 5: note = wherein X is any amino acid but preferably S, and it is preferably replaced by N or T
  VARIANT 10: note = wherein X is any amino acid but preferably V, and it is preferably replaced by G
LXSLXSVVTXP

SEQ ID NO: 165 - protein, 11 aa, synthetic construct
  VARIANT 2: note = wherein X is any amino acid but preferably Y, and it is preferably replaced by A or G or I or N or S or T or V or W
  VARIANT 5: note = wherein X is any amino acid but preferably S, and it is preferably replaced by N or T
  VARIANT 10: note = wherein X is any amino acid but preferably V, and it is preferably replaced by G
LXSLXSVVEXP

SEQ ID NO: 166 - protein, 11 aa, synthetic construct
  VARIANT 2: note = wherein X is any amino acid but preferably Y, and it is preferably replaced by A or G or I or N or S or T or V or W
  VARIANT 5: note = wherein X is any amino acid but preferably S, and it is preferably replaced by N or T
  VARIANT 10: note = wherein X is any amino acid but preferably V, and it is preferably replaced by G
LXSLXSVVDXP

SEQ ID NO: 167 - protein, 98 aa, synthetic construct
  VARIANT 58: note = wherein X is any amino acid but preferably T, and it is preferably replaced by E or A or I
EVQLLESGGGLVQPGGSLRLSCAASGFTFSSYAMSWVRQAPGKGLEWVSAISGSGGSXYYADSVKGRFTISRDNSKNTLYLQMNSLRAEDTAVYYCAK

SEQ ID NO: 168 - protein, 28 aa, synthetic construct
  VARIANT 18: note = wherein X is any amino acid but preferably T, and it is preferably replaced by E or A or I
PGKGLEWVSAISGSGGSXYYADSVKGRF

SEQ ID NO: 169 - protein, 17 aa, synthetic construct
  VARIANT 12: note = wherein X is any amino acid but preferably T, and it is preferably replaced by E or A or I
WVSAISGSGGSXYYADS

SEQ ID NO: 170 - protein, 10 aa, synthetic construct
  VARIANT 9: note = wherein X is any amino acid but preferably T, and it is preferably replaced by E or A or I
AISGSGGSXY

SEQ ID NO: 171 - protein, 8 aa, synthetic construct
  VARIANT 8: note = wherein X is any amino acid but preferably T, and it is preferably replaced by E or A or I
ISGSGGSX

SEQ ID NO: 172 - protein, 103 aa, synthetic construct
  VARIANT 51..53: note = wherein X is any amino acid
  VARIANT 54..62: note = wherein X is any amino acid or is absent
  VARIANT 63: note = wherein X is any amino acid but preferably T, and it is preferably replaced by E or A or I
EVQLLESGGGLVQPGGSLRLSCAASGFTFSSYAMSWVRQAPGKGLEWVSAXXXXXXXXXXXXXYYADSVKGRFTISRDNSKNTLYLQMNSLRAEDTAVYYCAK

SEQ ID NO: 173 - protein, 33 aa, synthetic construct
  VARIANT 11..13: note = wherein X is any amino acid
  VARIANT 14..22: note = wherein X is any amino acid or is absent
  VARIANT 23: note = wherein X is any amino acid but preferably T, and it is preferably replaced by E or A or I
PGKGLEWVSAXXXXXXXXXXXXXYYADSVKGRF

SEQ ID NO: 174 - protein, 22 aa, synthetic construct
  VARIANT 5..7: note = wherein X is any amino acid
  VARIANT 8..16: note = wherein X is any amino acid or is absent
  VARIANT 17: note = wherein X is any amino acid but preferably T, and it is preferably replaced by E or A or I
WVSAXXXXXXXXXXXXXYYADS

SEQ ID NO: 175 - skipped

SEQ ID NO: 176 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
  VAR_SEQ 18: note = wherein X is any amino acid but preferably T or S, and it is preferably replaced by A
TFGQGTKVXIKRXVXAPXVFIFPPSDEQL

SEQ ID NO: 177 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
  VARIANT 18: note = wherein X is any amino acid but preferably T or S, and it is preferably replaced by A
TFGQGTKLXIKRXVXAPXVFIFPPSDEQL

SEQ ID NO: 178 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
  VARIANT 18: note = wherein X is any amino acid but preferably T or S, and it is preferably replaced by A
TFGPGTKVXIKRXVXAPXVFIFPPSDEQL

SEQ ID NO: 179 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
  VARIANT 18: note = wherein X is any amino acid but preferably T or S, and it is preferably replaced by A
TFGGGTKVXIKRXVXAPXVFIFPPSDEQL

SEQ ID NO: 180 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
  VARIANT 18: note = wherein X is any amino acid but preferably T or S, and it is preferably replaced by A
TFGQGTRLXIKRXVXAPXVFIFPPSDEQL

SEQ ID NO: 181 - protein, 12 aa, synthetic construct
  VARIANT 2: note = wherein X is any amino acid but preferably I, and it is preferably replaced by A or G or H or N or P or Q or S or T
  VARIANT 6: note = wherein X is any amino acid but preferably T, and it is preferably replaced by P
  VARIANT 8: note = wherein X is any amino acid but preferably A, and it is preferably replaced by R or T or W or V
  VARIANT 11: note = wherein X is any amino acid but preferably T or S, and it is preferably replaced by A
VXIKRXVXAPXV

SEQ ID NO: 182 - protein, 12 aa, synthetic construct
  VARIANT 2: note = wherein X is any amino acid but preferably I, and it is preferably replaced by A or G or H or N or P or Q or S or T
  VARIANT 6: note = wherein X is any amino acid but preferably T, and it is preferably replaced by P
  VARIANT 8: note = wherein X is any amino acid but preferably A, and it is preferably replaced by R or T or W or V
  VARIANT 11: note = wherein X is any amino acid but preferably T or S, and it is preferably replaced by A
LXIKRXVXAPXV

SEQ ID NO: 183 - protein, 8 aa, synthetic construct
  VARIANT 2: note = wherein X is any amino acid but preferably T, and it is preferably replaced by P
  VARIANT 4: note = wherein X is any amino acid but preferably A, and it is preferably replaced by R or T or W or V
  VARIANT 7: note = wherein X is any amino acid but preferably T or S, and it is preferably replaced by A
RXVXAPXV

SEQ ID NO: 184 - protein, 6 aa, synthetic construct
  VARIANT 2: note = wherein X is any amino acid but preferably A, and it is preferably replaced by R or T or W or V
  VARIANT 5: note = wherein X is any amino acid but preferably T or S, and it is preferably replaced by A
VXAPXV

SEQ ID NO: 185 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGTGTKVTVLGQXKXNPXVTLFPPSSEEL

SEQ ID NO: 186 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGTGTKVTVLGQXKXAPXVTLFPPSSEEL

SEQ ID NO: 187 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGGGTKLTVLGQXKXNPXVTLFPPSSEEL

SEQ ID NO: 188 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGGGTKLTVLGQXKXAPXVTLFPPSSEEL

SEQ ID NO: 189 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGGGTQLIILGQXKXNPXVTLFPPSSEEL

SEQ ID NO: 190 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGGGTQLIILGQXKXAPXVTLFPPSSEEL

SEQ ID NO: 191 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGEGTELTVLGQXKXNPXVTLFPPSSEEL

SEQ ID NO: 192 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGEGTELTVLGQXKXAPXVTLFPPSSEEL

SEQ ID NO: 193 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 18: note = wherein X is any amino acid but preferably T or S, and it is preferably replaced by A
VFGGGTQLTVLGQXKXNPXVTLFPPSSEEL

SEQ ID NO: 194 - protein, 30 aa, synthetic construct
  VARIANT 14: note = wherein X is any amino acid but preferably T, and it is preferably replaced by P
  VARIANT 16: note = wherein X is any amino acid but preferably A, and it is preferably replaced by R or T or W or V
  VARIANT 19: note = wherein X is any amino acid but preferably T or S, and it is preferably replaced by A
VFGGGTQLTVLGQXKXAPXVTLFPPSSEEL

SEQ ID NO: 195 - protein, 9 aa, synthetic construct
  VARIANT 3: note = wherein X is any amino acid but preferably T, and it is preferably replaced by P
  VARIANT 5: note = wherein X is any amino acid but preferably A, and it is preferably replaced by R or T or W or V
  VARIANT 8: note = wherein X is any amino acid but preferably T or S, and it is preferably replaced by A
GQXKXNPXV

SEQ ID NO: 196 - protein, 9 aa, synthetic construct
  VARIANT 3: note = wherein X is any amino acid but preferably T, and it is preferably replaced by P
  VARIANT 5: note = wherein X is any amino acid but preferably A, and it is preferably replaced by R or T or W or V
  VARIANT 8: note = wherein X is any amino acid but preferably T or S, and it is preferably replaced by A
GQXKXAPXV

SEQ ID NO: 197 - protein, 6 aa, synthetic construct
  VARIANT 2: note = wherein X is any amino acid but preferably A, and it is preferably replaced by R or T or W or V
  VARIANT 5: note = wherein X is any amino acid but preferably T or S, and it is preferably replaced by A
KXNPXV

SEQ ID NO: 198 - protein, 6 aa, synthetic construct
  VARIANT 2: note = wherein X is any amino acid but preferably A, and it is preferably replaced by R or T or W or V
  VARIANT 5: note = wherein X is any amino acid but preferably T or S, and it is preferably replaced by A
KXAPXV

SEQ ID NO: 199 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 16: note = wherein X is any amino acid but preferably T, and it is preferably replaced by E or D
  VARIANT 17: note = wherein X is any amino acid but preferably V, and it is preferably replaced by G
AVLQSSGLXSLXSVVXXPSSSLGTQTYICNV

SEQ ID NO: 200 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 16: note = wherein X is any amino acid but preferably T, and it is preferably replaced by E or D
  VAR_SEQ 17: note = wherein X is any amino acid but preferably V, and it is preferably replaced by G
AVLQSSGLXSLXSVVXXPSSNFGTQTYTCNV

SEQ ID NO: 201 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 16: note = wherein X is any amino acid but preferably T, and it is preferably replaced by E or D
  VARIANT 17: note = wherein X is any amino acid but preferably V, and it is preferably replaced by G
AVLQSSGLXSLXSVVXXPSSSLGTQTYTCNV

SEQ ID NO: 202 - protein, 31 aa, synthetic construct
  VARIANT 9: note = wherein X is any amino acid but preferably Y, and it is preferably replaced by A or G or I or N or S or T or V or W
  VARIANT 12: note = wherein X is any amino acid but preferably S, and it is preferably replaced by N or T
  VARIANT 16: note = wherein X is any amino acid but preferably T, and it is preferably replaced by E or D
  VARIANT 17: note = wherein X is any amino acid but preferably V, and it is preferably replaced by G
AVLQSSGLXSLXSVVXXPSSSLGTKTYTCNV

SEQ ID NO: 203 - protein, 11 aa, synthetic construct
  VARIANT 2: note = wherein X is any amino acid but preferably Y, and it is preferably replaced by A or G or I or N or S or T or V or W
  VARIANT 5: note = wherein X is any amino acid but preferably S, and it is preferably replaced by N or T
  VARIANT 9: note = wherein X is any amino acid but preferably T, and it is preferably replaced by E or D
  VARIANT 10: note = wherein X is any amino acid but preferably V, and it is preferably replaced by G
LXSLXSVVXXP

SEQ ID NO: 204 - protein, 31 aa, synthetic construct
  UNSURE 9: note = wherein X marks the core amino acids, which are predicted to interact with the MHC II protein and can be mutated to reduce immunogenicity risks
  UNSURE 12: note = wherein X marks the core amino acids, which are predicted to interact with the MHC II protein and can be mutated to reduce immunogenicity risks
  UNSURE 14: note = wherein X marks the core amino acids, which are predicted to interact with the MHC II protein and can be mutated to reduce immunogenicity risks
  UNSURE 17: note = wherein X marks the core amino acids, which are predicted to interact with the MHC II protein and can be mutated to reduce immunogenicity risks
AVLQSSGLXSLXSXVEXPSSSLGTQTYICNV

SEQ ID NO: 205 - protein, 29 aa, synthetic construct
  UNSURE 10: note = wherein X marks the core amino acids, which are predicted to interact with the MHC II protein and can be mutated to reduce immunogenicity risks
  UNSURE 13: note = wherein X marks the core amino acids, which are predicted to interact with the MHC II protein and can be mutated to reduce immunogenicity risks
  UNSURE 15: note = wherein X marks the core amino acids, which are predicted to interact with the MHC II protein and can be mutated to reduce immunogenicity risks
TFGAGTKLEXKRXVXAPAVFIFPPSDEQL

SEQ ID NO: 206 - protein, 29 aa, synthetic construct
TFGAGTKLEIKRTVAAPSVFIFPPSDEQL

SEQ ID NO: 207 - protein, 16 aa, synthetic construct
TKLEIKRTVAAPAVFI

SEQ ID NO: 208 - protein, 19 aa, synthetic construct
TKLEIKRTVAAPAVFIFPP

SEQ ID NO: 209 - protein, 18 aa, synthetic construct
SGTASVVCLLKNFYPREA

SEQ ID NO: 210 - protein, 15 aa, synthetic construct
GTASVVCLLKNFYPR

SEQ ID NO: 211 - protein, 14 aa, synthetic construct
TASVVCLLKNFYPR

SEQ ID NO: 212 - protein, 15 aa, synthetic construct
SSGLYSLSSVVEVPS

SEQ ID NO: 213 - protein, 16 aa, synthetic construct
SSGLYSLSSVVEVPSS

SEQ ID NO: 214 - protein, 14 aa, synthetic construct
SGLYSLSSVVEVPS

SEQ ID NO: 215 - protein, 15 aa, synthetic construct
SGLYSLSSVVEVPSS

SEQ ID NO: 216 - protein, 19 aa, synthetic construct
EQDSKDSTYSLWSTLTLSK

SEQ ID NO: 217 - protein, 20 aa, synthetic construct
EQDSKDSTYSLWSTLTLSKA

SEQ ID NO: 218 - protein, 21 aa, synthetic construct
EQDSKDSTYSLWSTLTLSKAD

SEQ ID NO: 219 - protein, 19 aa, synthetic construct
QDSKDSTYSLWSTLTLSKA

SEQ ID NO: 220 - protein, 17 aa, synthetic construct
DSKDSTYSLWSTLTLSK

SEQ ID NO: 221 - protein, 18 aa, synthetic construct
DSKDSTYSLWSTLTLSKA

SEQ ID NO: 222 - protein, 19 aa, synthetic construct
DSKDSTYSLWSTLTLSKAD

SEQ ID NO: 223 - protein, 20 aa, synthetic construct
DSKDSTYSLWSTLTLSKADY

SEQ ID NO: 224 - protein, 17 aa, synthetic construct
SKDSTYSLWSTLTLSKA

SEQ ID NO: 225 - protein, 18 aa, synthetic construct
SKDSTYSLWSTLTLSKAD

SEQ ID NO: 226 - protein, 13 aa, synthetic construct
KDSTYSLWSTLTL

SEQ ID NO: 227 - protein, 15 aa, synthetic construct
KDSTYSLWSTLTLSK

SEQ ID NO: 228 - protein, 16 aa, synthetic construct
KDSTYSLWSTLTLSKA

SEQ ID NO: 229 - protein, 17 aa, synthetic construct
KDSTYSLWSTLTLSKAD

SEQ ID NO: 230 - protein, 18 aa, synthetic construct
KDSTYSLWSTLTLSKADY

SEQ ID NO: 231 - protein, 14 aa, synthetic construct
DSTYSLWSTLTLSK

SEQ ID NO: 232 - protein, 15 aa, synthetic construct
DSTYSLWSTLTLSKA

SEQ ID NO: 233 - protein, 16 aa, synthetic construct
DSTYSLWSTLTLSKAD

SEQ ID NO: 234 - protein, 16 aa, synthetic construct
SGTASVVCLLKNFYPR

SEQ ID NO: 235 - protein, 17 aa, synthetic construct
SGTASVVCLLKNFYPRE

SEQ ID NO: 236 - protein, 18 aa, synthetic construct
SGTASVVCLLKNFYPREA

SEQ ID NO: 237 - protein, 24 aa, synthetic construct
SGTASVVCLLKNFYPREAKVQWKV

SEQ ID NO: 238 - protein, 15 aa, synthetic construct
GTASVVCLLKNFYPR

SEQ ID NO: 239 - protein, 14 aa, synthetic construct
TASVVCLLKNFYPR

SEQ ID NO: 240 - protein, 15 aa, synthetic construct
SSGLYSLSSVVEVPS

SEQ ID NO: 241 - protein, 16 aa, synthetic construct
SSGLYSLSSVVEVPSS

SEQ ID NO: 242 - protein, 17 aa, synthetic construct
SSGLYSLSSVVEVPSSS

SEQ ID NO: 243 - protein, 14 aa, synthetic construct
SGLYSLSSVVEVPS

SEQ ID NO: 244 - protein, 19 aa, synthetic construct
EQDSKDSTYSLWSTLTLSK

SEQ ID NO: 245 - protein, 20 aa, synthetic construct
EQDSKDSTYSLWSTLTLSKA

SEQ ID NO: 246 - protein, 21 aa, synthetic construct
EQDSKDSTYSLWSTLTLSKAD

SEQ ID NO: 247 - protein, 17 aa, synthetic construct
DSKDSTYSLWSTLTLSK

SEQ ID NO: 248 - protein, 18 aa, synthetic construct
DSKDSTYSLWSTLTLSKA

SEQ ID NO: 249 - protein, 19 aa, synthetic construct
DSKDSTYSLWSTLTLSKAS

SEQ ID NO: 250 - protein, 20 aa, synthetic construct
DSKDSTYSLWSTLTLSKADY

SEQ ID NO: 251 - protein, 17 aa, synthetic construct
SKDSTYSLWSTLTLSKA

SEQ ID NO: 252 - protein, 18 aa, synthetic construct
SKDSTYSLWSTLTLSKAD

SEQ ID NO: 253 - protein, 15 aa, synthetic construct
KDSTYSLWSTLTLSK

SEQ ID NO: 254 - protein, 16 aa, synthetic construct
KDSTYSLWSTLTLSKA

SEQ ID NO: 255 - protein, 17 aa, synthetic construct
KDSTYSLWSTLTLSKAD

SEQ ID NO: 256 - protein, 18 aa, synthetic construct
KDSTYSLWSTLTLSKADY

SEQ ID NO: 257 - protein, 14 aa, synthetic construct
DSTYSLWSTLTLSK

SEQ ID NO: 258 - protein, 15 aa, synthetic construct
DSTYSLWSTLTLSKA

SEQ ID NO: 259 - protein, 16 aa, synthetic construct
DSTYSLWSTLTLSKAD

SEQ ID NO: 260 - protein, 13 aa, synthetic construct
TYSLWSTLTLSKA

SEQ ID NO: 261 - protein, 682 aa, synthetic construct
EVQLLESGGGLVQPGGSLRLSCAASGFTFSSYAMSWVRRAPGKGLEWVSGIGSYGGGTYYADSVKGRFTISRDNSKNTLYLQMNSLRAEDTAVYYCARYVNFGMDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSELVMTQSPSSLTVTAGEKVTMSCKSSQSLLNSGNQKNYLTWYQRKPGQPPKLLIYWASTRESGVPDRFTGSGSGTDFTLTISSVQAEDLAVYYCQNDYSYPLTFGAGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 223 aa, synthetic construct
EVQLLEQSGAELVRPGTSVKISCKASGYAFTNYWLGWVKERPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSC

SEQ ID NO: 263 - protein, 214 aa, synthetic construct
DIQMTQSPSSLSASVGDRVTITCRASQSISSYLNWYQEKPGKAPKLLIYAASSLQSGVPSRFSGSGSGTDFTLTISSLQPEDFATYYCQQYGRNPPTFGQGTKLEIKRTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 264 - protein, 677 aa, synthetic construct
EVQLLESGGGLVQPGGSLRLSCAASGFTFSSYAMSWVRRAPGKGLEWVSGIGSYGGGTYYADSVKGRFTISRDNSKNTLYLQMNSLRAEDTAVYYCARYVNFGMDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQSISSYLNWYQRKPGKAPKLLIYAASSLQSGVPSRFSGSGSGTDFTLTISSLQPEDFATYYCQQSSHGPLLTFGQGTKLEIKRPVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 221 aa, synthetic construct
QVQLVQSGAEVKKPGSSVKVSCKASGGTFGYYAIHWVREAPGQGLEWMGGIGSIFGTANYAQKFQGRVTITADESTSTAYMELSSLRSEDTAVYYCARAWSSDHMDYWGQGTLVTVSSASTKGPSVFPLAPSSKSTSGGTAALGCLVKDYFPEPVTVSWNSGALTSGVHTFPAVLQSSGLYSLTSVVEVPSSSLGTQTYICNVNHKPSNTKVDKKVEPKSC

SEQ ID NO: 266 - protein, 214 aa, synthetic construct; identical to SEQ ID NO: 263

SEQ ID NO: 267 - protein, 680 aa, synthetic construct
EVQLLESGGGLVQPGGSLRLSCAASGFTFSSYAMSWVRRAPGKGLEWVSAISGSGGSTYYADSVKGRFTISRDNSKNTLYLQMNSLRAEDTAVYYCARYYGGYYSAWMDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQSISSYLNWYQRKPGKAPKLLIYAASSLQSGVPSRFSGSGSGTDFTLTISSLQPEDFATYYCQQYGRNPPTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 214 aa, synthetic construct
DIQMTQSPSSLSASVGDRVTITCRASQSISSYLNWYQEKPGKAPKLLIYAASSLQSGVPSRFSGSGSGTDFTLTISSLQPEDFATYYCQQTYGYLHTFGQGTKLEIKRTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 269 - protein, 220 aa, synthetic construct
EVQLLESGGGLVQPGGSLRLSCAASGFTFSSYAMSWVREAPGKGLEWVSGIGSYGGGTYYADSVKGRFTISRDNSKNTLYLQMNSLRAEDTAVYYCARYVNFGMDYWGQGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSC

SEQ ID NO: 270 - protein, 678 aa, synthetic construct
EVQLLESGGGLVQPGGSLRLSCAASGFTFSTYGMHWVRRAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQSISSYLNWYQRKPGKAPKLLIYAASSLQSGVPSRFSGSGSGTDFTLTISSLQPEDFATYYCQQTYGYLHTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 217 aa, synthetic construct
QSVLTQPPSASGTPGQRVTISCTGSSSNIGAGYNVYWYQELPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTECS

SEQ ID NO: 272 - protein, 224 aa, synthetic construct
EVQLLESGGGLVQPGGSLRLSCAASGFTFSSYAMSWVREAPGKGLEWVSAISGSGGSTYYADSVKGRFTISRDNSKNTLYLQMNSLRAEDTAVYYCARYYGGYYSAWMDYWGQGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSC

SEQ ID NO: 273 - protein, 676 aa, synthetic construct
EVQLLESGGGLVQPGGSLRLSCAASGFTFSSYAMSWVRRAPGKGLEWVSGIGSYGGGTYYADSVKGRFTISRDNSKNTLYLQMNSLRAEDTAVYYCARYVNFGMDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDIQMTQSPSSLSASVGDRVTITCRASQSISSYLNWYQRKPGKAPKLLIYAASSLQSGVPSRFSGSGSGTDFTLTISSLQPEDFATYYCQQTYGYLHTFGQGTKLEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 214 aa, synthetic construct; identical to SEQ ID NO: 263

SEQ ID NO: 275 - protein, 224 aa, synthetic construct; identical to SEQ ID NO: 272

SEQ ID NO: 276 - protein, 683 aa, synthetic construct
EVQLLESGGGLVQPGGSLRLSCAASGFTFSSSYMGWVRRAPGKGLEWVSSIGSGSYSTSYADSVKGRFTISRDNSKNTLYLQMNSLRAEDTAVYYCARYPSVPFPLHLDYWGQGTLVTVSSASTKGPSVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCTGSSSNIGAGYNVYWYQRLPGTAPKLLIYGNINRPSGVPDRFSGSKSGTSASLAISGLRSEDEADYYCAAWDKSISGLVFGGGTKLTVLGQPKAAPAVTLFPPSSEELQANKATLVCLIKDFYPGAVTVAWKADSSPVKAGVETTTPSKQSNNKYAAWSYLSLTPEQWKSHRSYSCQVTHEGSTVEKTVAPTECS

SEQ ID NO: 277 - protein, 213 aa, synthetic construct
DIQMTQSPSSLSASVGDRVTITCRASQSISSYLNWYQEKPGKAPKLLIYAASSLQSGVPSRFSGSGSGTDFTLTISSLQPEDFATYYCQQAGNPHTFGQGTKLEIKRTVAAPSVFIFPPSDEQLKSGTASVVCYLNNFYPREAKVQWKVDNALQSGNSQESVTEQDSKDSTYSLWSTLTLSKADYEKHKVYACEVTHQGLSSPVTKSFNRGEC

SEQ ID NO: 278 - protein, 222 aa, synthetic construct
EVQLLESGGGLVQPGGSLRLSCAASGFTFSTYGMHWVREAPGKGLEWLSYISGGSSYIFYADSVKGRFTISRDNSKNTLYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLTSVVEVPSSSLGTQTYICNVNHKPSNTKVDKKVEPKSC

SEQ ID NO: 279 - protein, 683 aa, synthetic construct; identical to SEQ ID NO: 276

SEQ ID NO: 280 - protein, 213 aa, synthetic construct; identical to SEQ ID NO: 277

SEQ ID NO: 281 - protein, 222 aa, synthetic construct
EVQLLESGGGLVQPGGSLRLSCAASGFTFSTYGMHWVREAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSC

SEQ ID NO: 282 - protein, 447 aa, synthetic construct
EVQLLESGGGLVQPGGSLRLSCAASGFTFSSYAMSWVRQAPGKGLEWVSGIGSYGGGTYYADSVKGRFTISRDNSKNTLYLQMNSLRAEDTAVYYCARYVNFGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 283 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYGRNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 11 aa, synthetic construct
TFGAGTKLEIK

SEQ ID NO: 285 - protein, 29 aa, synthetic construct
  VARIANT 18: note = wherein X is any amino acid but preferably T or S, and it is preferably replaced by A
TFGAGTKLEIKRTVAAPXVFIFPPSDEQL

SEQ ID NO: 286 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V.
TFGAGTKLXIKRXVXAPAVFIFPPSDEQL

SEQ ID NO: 287 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGAGTKLXIKRXVXAPSVFIFPPSDEQL

SEQ ID NO: 288 - protein, 29 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
  VARIANT 18: note = wherein X is any amino acid but preferably T or S, and it is preferably replaced by A
TFGAGTKLXIKRXVXAPXVFIFPPSDEQL

SEQ ID NO: 289 - protein, 9 aa, synthetic construct
  VARIANT 8: note = wherein X is any amino acid but preferably T or S, and it is preferably replaced by A
GQPKAAPXV

SEQ ID NO: 290 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 291 - protein, 98 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 292 - protein, 98 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 293 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 294 - protein, 16 aa, synthetic construct
  VARIANT 9: note = wherein X is any amino acid but preferably I, and it is preferably replaced by A or G or H or N or P or Q or S or T
  VARIANT 13: note = wherein X is any amino acid but preferably T, and it is preferably replaced by P
  VARIANT 15: note = wherein X is any amino acid but preferably A, and it is preferably replaced by R or T or W or V
TFGAGTKLXIKRXVXA

SEQ ID NO: 295 - protein, 6 aa, synthetic construct
  VARIANT 5..6: note = wherein SG is repeated 1 to 5 times or is absent
SGSGSG

SEQ ID NO: 296 - protein, 7 aa, synthetic construct
  VARIANT 2: note = wherein X is any amino acid
  VARIANT 4: note = wherein X is any amino acid
RXVXAPA

SEQ ID NO: 297 - protein, 8 aa, synthetic construct
  VARIANT 3: note = wherein X is any amino acid
  VARIANT 5: note = wherein X is any amino acid
GQXKXNPA

SEQ ID NO: 298 - protein, 8 aa, synthetic construct
  VARIANT 3: note = wherein X is any amino acid
  VARIANT 5: note = wherein X is any amino acid
GQXKXAPA

SEQ ID NO: 299 - protein, 9 aa, synthetic construct
GPSNDQEKR

(19) United States
(12) Patent Application Publication
(10) Pub. No.: US 2026/0001961 A1
(43) Pub. Date: Jan. 1, 2026

(54) NOVEL POLYPEPTIDES

(71) Applicant: ALLIGATOR BIOSCIENCE AB, Lund (SE)

(72) Inventors: Laura von Schantz, Lund (SE); Barnabas NYESIGA, Lund (SE); Mattias Levin, Lund (SE); Anna SÄLL, Lund (SE)

(21) Appl. No.: 18/703,623

(22) PCT Filed: Nov. 4, 2022
(86) PCT No.: PCT/EP22/80860

§ 371 Date: Apr. 22, 2024

(30) Foreign Application Priority Data
Nov. 5, 2021 (GB) 2115925.6
Mar. 30, 2022 (GB) 2204539.7
Sep. 2, 2022 (GB) 2212801.1

(51) Int. Cl. C07K 16/30; A61P 35/00; C07K 16/28

(52) U.S. Cl. CPC C07K 16/30; A61P 35/00; C07K 16/2875; C07K 2317/31; C07K 2317/92

(57) ABSTRACT

The present invention relates to polypeptides (in particular, bispecific antibodies) including new mutations that reduce immunogenicity and/or improve purity during manufacture.

This application is a § 371 application of PCT/EP2022/080860, filed Nov. 4, 2022, which claims priority to GB 2115925.6, filed Nov. 5, 2021; GB 2204539.7, filed Mar. 30, 2022; and GB 2212801.1, filed Sep. 2, 2022. The foregoing applications are incorporated by reference herein.

Incorporated herein by reference in its entirety is the Sequence Listing being concurrently submitted as a XML file named SeqList, created Apr. 21, 2024, and having a size of 429,369 bytes.

### FIELD OF THE INVENTION

The present invention relates to polypeptides (in particular, bispecific antibodies) including new mutations that reduce immunogenicity and/or improve purity during manufacture.

### BACKGROUND TO THE INVENTION

Cancer is a leading cause of premature deaths in the developed world. Immunotherapy of cancer aims to mount an effective immune response against tumour cells. This may be achieved by, for example, breaking tolerance against tumour antigen, augmenting anti-tumour immune responses, and stimulating local cytokine responses at the tumour site. The key effector cell of a long lasting anti-tumour immune response is the activated tumour-specific effector T cell. Potent expansion of activated effector T cells can redirect the immune response towards the tumour. In this context, regulatory T cells (Treg) play a role in inhibiting the anti-tumour immunity. Depleting, inhibiting, reverting or inactivating Tregs may therefore provide anti-tumour effects and revert the immune suppression in the tumour microenvironment. Further, incomplete activation of effector T cells by, for example, dendritic cells can cause T cell anergy, which results in an inefficient anti-tumour response, whereas adequate induction by dendritic cells can generate a potent expansion of activated effector T cells, redirecting the immune response towards the tumour. In addition, natural killer (NK) cells play an important role in tumour immunology by attacking tumour cells with down-regulated human leukocyte antigen (HLA) expression and by inducing antibody-dependent cellular cytotoxicity (ADCC). Stimulation of NK cells may thus also reduce tumour growth.

Therapeutic polypeptides (and, in particular, bispecific antibodies) have utility in cancer treatment as they allow for dual targeting of cells. For example, bispecific antibodies may be capable of activating the host immune cells in the vicinity of tumour cells and are thus an alternative to existing monospecific drugs that target only one antigen.

Bispecific antibodies are more difficult to generate compared to monoclonal antibodies. Many different formats have been invented and all formats have different strengths and limitations.

Challenging to developing bispecific antibodies is designing structures and amino acid sequences that are practical to produce at a good yield and purity, whilst avoiding any modifications (particularly, mutations) that lead to increased immunogenicity which would reduce the suitability for therapeutic use.

Against this background, the inventors have surprisingly identified polypeptide mutations that reduce immunogenicity and/or improve the purity during manufacture.

### SUMMARY OF INVENTION

The following invention provides new mutations for polypeptides (in particular, bispecific antibodies, preferably of the RUBY format structure shown in FIG. 11). These mutations (the “secondary mutations” described herein) further reduce the immunogenicity of other mutations introduced into the antibody (the “primary mutations” described herein) and/or improve the purity and the ease of manufacturing of the polypeptides (the “improved purity mutations” described herein).

It should be noted that the new mutations described herein further greatly improve the properties of the polypeptides to which they are applied, which themselves may already have a good immunogenicity profile and ease of manufacture. For example, the exemplified polypeptides to which the new mutations are applied were already superior to many previously available and safe for the clinic. The new mutations described herein improve those excellent previous characteristics. Based on this, these new mutations can be referred to as being “optimised”.

### DETAILED DESCRIPTION OF THE INVENTION

In a first aspect, the invention provides a polypeptide having specificity for an antigen, wherein the polypeptide comprises a heavy chain region and a light chain region;
i. wherein the polypeptide comprises one or more primary mutations to promote an association of the heavy chain region with the light chain region, and wherein such mutations increase the predicted risk of inducing immunogenic responses immunogenicity and/or increase the immunogenicity of the polypeptide; and
ii. wherein the polypeptide comprises one or more secondary mutations to reduce or avoid the predicted risk of inducing immunogenic responses and/or reduce or avoid the predicted risk immunogenicity caused by the one or more primary mutations.

The term “polypeptide” is used herein in its broadest sense to refer to a compound of two or more subunit amino acids, amino acid analogues, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogues and peptidomimetics.

In a preferred embodiment, the polypeptide is an antibody.

In a further preferred embodiment, the polypeptide is a bispecific polypeptide, more preferably a bispecific antibody. In an alternative embodiment, the polypeptide is a monospecific polypeptide.

As used herein, the terms “antibody” or “antibodies” refer to molecules that contain an antigen binding site, e.g. immunoglobulin molecules and immunologically active fragments of immunoglobulin molecules that contain an antigen binding site.

Immunoglobulin molecules can be of any type (e.g. IgG, IgE, IgM, IgD, IgA and IgY), class (e.g. IgG1, IgG2, IgG3, IgG4, IgA1 and IgA2) or a subclass of immunoglobulin molecule. Antibodies include, but are not limited to, synthetic antibodies, monoclonal antibodies, single domain antibodies, single chain antibodies, recombinantly produced antibodies, multi-specific antibodies (including bispecific antibodies), human antibodies, humanized antibodies, chimeric antibodies, intrabodies, scFvs (e.g. including mono-specific and bi-specific, etc.), Fab fragments, F(ab′) fragments, disulfide-linked Fvs (sdFv), anti-idiotypic (anti-Id) antibodies, and epitope-binding fragments of any of the above.

The term “bispecific” as used herein means the polypeptide is capable of specifically binding two distinct target entities. Bispecific polypeptides, e.g. antibodies, targeting two targets, have the potential to induce specific activation of the immune system in locations where both targets are over expressed.

The term antibody “having specificity for” refers to an antibody that is constructed to direct its binding specificity(ies) at a certain target/marker/epitope/antigen, i.e. an antibody that immunospecifically binds to a target/marker/epitope/antigen. Also, the expressions antibodies “selective for”, “directed to”, “binding”, “against” or “directed against” a certain target/marker/epitope/antigen may be used, having the same definition as “having specificity for”. A bispecific antibody having specificity for two different targets/markers/epitopes/antigens binds immunospecifically to both targets/markers/epitopes/antigens. If an antibody is directed to a certain target antigen, such as CD40, it is thus assumed that said antibody could be directed to any suitable epitope present on said target antigen structure.

One skilled in immunology would understand and appreciate the concept of to “promote an association of the heavy chain region with the light chain region”. Here, we include that the primary mutations prevent incorrect binding of the heavy chain region and the light chain region, i.e. the mutations prevent the formation of manufacturing by-products. Alternatively, or additionally, the primary mutations may prevent self-aggregation of the heavy chain region and the light chain region i.e. the mutations prevent the formation of aggregates. It will be appreciated by persons skilled in the art, that in one embodiment the primary mutations may prevent the formation of aggregates and/or a by-products by generating steric hindrance and/or incompatibility between charges—these terms are discussed in further detail below.

The term “immunogenicity” would be well understood by one skilled in immunology, and we include the characteristics of the polypeptide that cause it to stimulate an unwanted immune response, directed against the said polypeptide, in an individual, to whom the polypeptide has been administered.

In a preferred embodiment, the increase in the immunogenicity is characterised as a higher predicted immunogenicity risk when compared to the predicted immunogenicity risk of a primary mutation control polypeptide and/or a higher immunogenicity when compared to the immunogenicity of a primary mutation control polypeptide, preferably the primary mutation control polypeptide does not comprise the one or more primary mutations.

In a particular embodiment, the primary mutation control polypeptide comprises or consists of an amino acid sequence that is the same as the polypeptide prior to the one or more primary mutations being introduced.

In a preferred embodiment, the reduction in the immunogenicity is characterised as a lower predicted immunogenicity risk when compared to the predicted immunogenicity risk of a first secondary mutation control polypeptide and/or a lower immunogenicity when compared to the immunogenicity of a first secondary mutation control polypeptide, preferably the first secondary mutation control polypeptide comprises the one or more primary mutations but does not comprise the one or more secondary mutations.

In a particular embodiment, the first secondary mutation control polypeptide comprises or consists of an amino acid sequence that is the same as the polypeptide comprising one or more of the primary mutations prior to the one or more secondary mutations being introduced.

In a preferred embodiment, the avoidance of immunogenicity is characterised as a similar, or the same, predicted immunogenicity risk when compared to the predicted immunogenicity risk of a second secondary mutation control polypeptide and/or immunogenicity when compared to the immunogenicity of a second secondary mutation control polypeptide, preferably the second secondary mutation control polypeptide does not comprise of the one or more primary mutations or the one or more secondary mutations.

In a particular embodiment, the second secondary mutation control polypeptide comprises or consists of an amino acid sequence that is the same as the polypeptide prior to the one or more primary mutations being introduced and prior to the one or more secondary mutations being introduced.

As explained in the Examples (in particular, Example 1), one method of predicting or identifying the immunogenicity of a polypeptide is to conduct in silico predictions of whether its amino acid sequence will bind to molecules and/or receptors in an individual that will lead to an unwanted immune response, such as considering whether the amino acid sequence would bind to major histocompatibility complex II (MHCII). This work could be conducted using the AbEpiAnalyzer tool (EIR Sciences), amongst other techniques. It would be appreciated by one skilled in immunology and/or computational biology how to conduct such predictions within the embodiments mentioned above, such as comparing amino acid sequences of the polypeptide of the invention with a primary mutation control polypeptide, a first secondary mutation control polypeptide, and/or a second secondary mutation control polypeptide.

As would also be appreciated, in vivo and/or in vitro experiments regarding the immunogenicity of the polypeptides of the invention (including in conjunction with the control polypeptides discussed herein) could also be conducted; for example, using appropriate model organisms.

In one embodiment, the immunogenicity is antigen-presenting cell (APC) immunogenicity and/or T-cell dependent immunogenicity.

The terms antigen-presenting cells (APCs) and T-cells would be known to one skilled in immunology; in particular in relation to APCs these are cells that process and present antigens within the context of MHC. The APCs are then able to present those antigens to other immune cells, such as T-cells.

In a particular embodiment, the APC is one or more APC selected from the list comprising of: a dendritic cell; a macrophage; or a B cell, preferably a dendritic cell.

In a preferred embodiment, the secondary mutations reduce or avoid the polypeptide binding to an MHC class II and/or a T-cell receptor (TCR), preferably to MHC class II.

The concept of a mutation causing a reduction or the avoidance of the binding of the polypeptide to MHC class II and/or a TCR would be appreciated and understood by one skilled in immunology. By “reduce the polypeptide binding”, we include that the affinity of the polypeptide with the secondary mutations for MHC class II and/or a TCR is lower than the polypeptide without those secondary mutations (such as the first secondary mutation control polypeptide described above). By “avoid the polypeptide binding”, we include that there is no detectable binding between the polypeptide with the secondary mutations and MHC class II and/or a TCR. An exemplary method for measuring binding affinity is a competition immunoassay (such as described in Wang et al., 2008 PLoS Comput Biol. 4(4): e1000048) or Octet, as described in the Examples.

In a second aspect, the invention provides a polypeptide having specificity for an antigen, wherein the polypeptide comprises a heavy chain variable domain (VH) comprising a protein A binding site, wherein the VH domain comprises a mutation that reduces or avoids the binding of protein A to the protein A binding site.

As would be known by one skilled in immunology, protein A was originally found in Staphylococcus aureus, and has a high affinity for a number of mammalian proteins including IgGs (such as the VH domain). Due to its affinity for IgG, protein A is often used to selectively isolate polypeptides during purification. Whilst this is a useful tool, if the polypeptide is complex, and particularly if it is made of multiple polypeptide chains such as a bispecific antibody described herein, protein A sites in the polypeptide can lead to co-purification of the complete polypeptide of interest with unwanted misfolded or unbound component polypeptide chains. As will be appreciated, this co-purification can lead to contamination with the unwanted polypeptide, which is undesirable. Accordingly, the inventors have identified that it can be desirable to mutate protein A binding sites in polypeptide regions that are particularly prone to forming unwanted polypeptides during the production process. The inventors have surprisingly found that mutating the protein A binding site in a VH domain can improve purity of polypeptides of interest during the production process. The mutations of the protein A binding site described herein are “optimised RUBY” mutations, and may also be referred to as “improved purity” mutations.

The concept of a mutation causing a reduction or the avoidance of the binding of protein A to a protein A binding site would be appreciated and understood by one skilled in immunology. By “reduces the binding of protein A to the protein A binding site”, we include that the affinity of protein A to the protein A binding site with the mutations is lower than the affinity of protein A to the protein A binding site without the mutations and/or that the mutations reduce the affinity of protein A to an extent that it no longer can be bound (and/or co-purified) by protein A-specific methods during antibody production. By “avoids the binding of protein A to the protein A binding site”, we include that there is no detectable binding between protein A and the protein binding site with the mutations. An exemplary method for measuring binding affinity is Octet, as described in the Examples.

In a particular embodiment, the mutation that reduces or avoids the binding of protein A to the protein A binding site is one or more mutations that reduce or avoid the binding of protein A to the protein A binding site.

In one embodiment of the second aspect of the invention, the VH is derived from the IGHV3 germline gene family.

A VH derived from the IGHV3 germline gene family has a protein A binding site with protein A binding domains making up that site being identified in conserved parts of framework regions 1, 3 and the CDRH2, so is particularly amenable to mutation to reduce or avoid protein A binding. This is further discussed in Bach, Lewis et al. 2015 J Chromatogr A.; 1409:60-9, Potter et al. 1996, J Immunol.; 157(7):2982-8. PMID: 8816406; and Potter et al 1997, Int Rev Immunol.; 14(4):291-308. doi: 10.3109/08830189709116521. PMID: 9186782.

In one embodiment, the mutation to the protein A binding site is in one or more of: the framework 1 region of a VH derived from the IGHV3 germline gene family; the framework 2 region of a VH derived from the IGHV3 germline gene family; and CDRH2 of a VH derived from the IGHV3 germline gene family, preferably the CDRH2 of a VH derived from the IGHV3 germline gene family.

An exemplary IGHV3 germline gene family member is IGHV3-23*01, which has the sequence of:

(CDR2 underlined)
SEQ ID NO: 1
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAK

In a preferred embodiment, the mutation to the protein A binding site is a mutation to the last amino acid (i.e. the C-terminal amino acid) of the CDRH2 of a VH derived from the IGHV3 germline gene family.

It is included that the mutations of the first aspect of the invention can be combined with the mutations of the second aspect of the invention, in the same polypeptide. Such potential combinations of the mutations discussed herein are disclosed below.

In a preferred embodiment of the first and second aspect, the polypeptide is a bispecific antibody and comprises:
(a) an immunoglobulin molecule having specificity for a first antigen, the immunoglobulin molecule comprising a first heavy chain region and a first light chain region; and
(b) at least one Fab fragment having specificity for a second antigen, the Fab fragment comprising a second heavy chain region and a second light chain region, wherein the second light chain region is fused to the C-terminus of the first heavy chain region,
and wherein the one or more primary mutations promote association of the first heavy chain region with the first light chain region and/or promote association of the second heavy chain region with the second light chain region. This particular embodiment includes a polypeptide having a RUBY format structure, as generally shown in FIG. 11.

It will therefore be appreciated by persons skilled in the art that the term “immunoglobulin” includes immunoglobulin molecules and immunologically active fragments of immunoglobulin molecules that contain an antigen binding site, as described above.

As used herein, the term “antibody fragment” is a portion of an antibody such as F(ab′)2, F(ab)2, Fab′, Fab, Fv, scFv and the like. Regardless of structure, an antibody fragment binds with the same antigen that is recognized by the intact antibody. For example, an anti-CD40 antibody fragment binds to CD40. The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues.

By “Fab fragment”, we include Fab fragments (comprising a complete light chain and the variable region and CH1 region of a heavy chain) which are capable of binding the same antigen that is recognized by the intact antibody. Fab fragment is a term known in the art, and Fab fragments comprise one constant and one variable domain of each of the heavy and the light chain.

In one embodiment of the invention, one of binding domain B1 or binding domain B2 is an immunoglobulin molecule, and one of binding domain B1 or binding domain B2 is a Fab fragment, wherein the Fab fragment is fused to the C terminus of the heavy chain of the immunoglobulin via the light chain of the Fab fragment.

For example, the polypeptide may have a format as shown in FIG. 11. Such a format is referred to as the “RUBY™ format” (as described in pending UK patent application 1820556.7 and the PCT application WO 2020/127354). Antibodies in the “RUBY™ format” and “optimised RUBY™ format”, as described herein, are particularly preferred, for the bispecific polypeptides of the invention.

In one embodiment, the one or more Fab fragment(s) is linked to the C-terminal end of the immunoglobulin via a linker.

In one embodiment of the invention, the bispecific polypeptide is tetravalent, capable of binding bivalently to each of the two antigens.

In one embodiment, the bispecific polypeptide comprises an immunoglobulin arranged as an antibody with two arms and therefore two binding sites for the first antigen, and two of the Fab fragments, each providing a binding site for the second antigen. Thus, there are two binding sites for the first antigen and two binding sites for the second antigen. The bispecific polypeptide of this embodiment may comprise three polypeptide chains: (1) chain H1 which comprises the heavy chain of the IgG a linker and the light chain of a Fab; (2) chain L1 is the light chain for the IgG; and (3) chain H2 is the heavy chain for the appended (attached) Fab. In a preferred embodiment, the bispecific polypeptide may comprise six polypeptide chains: (a) two chain H1, which comprise the heavy chain of the IgG a linker and the light chain of a Fab; (b) two chain L1, which are the light chain for the IgG; and (c) two chain H2, which are the heavy chain for the appended (attached) Fab. This structure can be used for both the “RUBY™ format” and “optimised RUBY™ format” antibodies, and these “H1”, “H2” and “L1” chains are described below with reference to specific mutations.

In one embodiment, binding domain B1 is an immunoglobulin and binding domain B2 is a Fab. In an alternative embodiment, binding domain B1 is a Fab and binding domain B2 is an immunoglobulin.

Thus, a first heavy chain polypeptide is a polypeptide comprising or consisting of the heavy chain of a first antibody or antigen-binding fragment, and a first light chain polypeptide is a polypeptide comprising or consisting of the light chain of a first antibody or antigen-binding fragment.

Accordingly, a second heavy chain polypeptide is a polypeptide comprising or consisting of the heavy chain of a second antibody or antigen-binding fragment, and a second light chain polypeptide is a polypeptide comprising or consisting of the light chain of a second antibody or antigen-binding fragment.

The first and second antibodies or antigen-binding fragments have specificity for a first antigen and a second antigen, respectively.

In one embodiment of the bispecific antibody of the invention, the immunoglobulin molecule comprises two copies of the first heavy chain polypeptide and/or two copies of the first light chain polypeptide.

Thus, in one embodiment the immunoglobulin molecule comprises two heavy chain polypeptides and two corresponding light chain polypeptides. By “corresponding” we mean that the heavy and light chains assemble into an antibody format, i.e. the heavy chains associate with the light chains via the CH1 and CKappa or CLambda regions, and the VH and VL regions, and the heavy chains are also linked together via the CH2 and CH3 regions.

In one embodiment of the bispecific antibody of the invention, the immunoglobulin molecule comprises two copies of the first heavy chain polypeptide and/or two copies of the first light chain polypeptide.

Thus, in one embodiment the immunoglobulin molecule comprises two heavy chain polypeptides and two corresponding light chain polypeptides. By “corresponding” we mean that the heavy and light chains assemble into an antibody format, i.e. the heavy chains associate with the light chains via the CH1 and CKappa or CLambda regions, and the VH and VL regions, and the heavy chains are also linked together via the CH2 and CH3 regions.

In one embodiment the bispecific antibody comprises two Fab fragments according to (b), i.e. two Fab fragments according to having specificity for a second antigen, the Fab fragments each comprising a second heavy chain polypeptide and a second light chain polypeptide.

Thus, in one embodiment the bispecific antibody comprises
(a) an immunoglobulin molecule with specificity for a first antigen, and comprising two heavy chain polypeptides and two light chain polypeptides, and
(b) two Fab fragments (with specificity for a second antigen) and comprising a second heavy chain polypeptide and a second light chain polypeptide

Accordingly, in one embodiment, the bispecific antibody comprises:
an immunoglobulin molecule comprising two copies of the first heavy chain polypeptide and two copies of the first light chain polypeptide,
and the bispecific antibody further comprises two Fab fragments according to having specificity for a second antigen, the Fab fragments each comprising a second heavy chain polypeptide and a second light chain polypeptide,
and the first Fab fragment is fused to the C-terminus of the first copy of the first heavy chain polypeptide via the light chain polypeptide of the Fab fragment,
and the second Fab fragment is fused to the C-terminus of the second copy of the first heavy chain polypeptide via the light chain polypeptide of the Fab fragment.

In one embodiment of the bispecific antibody according to first aspect of the invention, the immunoglobulin molecule comprises a human Fc region or a variant of a said region, where the region is an IgG1, IgG2, IgG3 or IgG4 region, preferably an IgG1 or IgG4 region.

The constant (Fc) regions of the antibodies may mediate the binding of the immunoglobulin to host tissues or factors, including various cells of the immune system (e.g. effector cells) and the first component (Clq) of the classical complement system. The Fc region is preferably a human Fc region, or a variant of a said region. The Fc region may be an IgG1, IgG2, IgG3 or IgG4 region, preferably an IgG1 or IgG4 region. A variant of an Fc region typically binds to Fc receptors, such as FcgammaR and/or neonatal Fc receptor (FcRn) with altered affinity providing for improved function and/or half-life of the polypeptide. The biological function and/or the half-life may be either increased or decreased relative to the half-life of a polypeptide comprising a native Fc region. Examples of such biological functions which may be modulated by the presence of a variant Fc region include antibody-dependent cell cytotoxicity (ADCC), antibody-dependent cellular phagocytosis (ADCP), complement-dependent cytotoxicity (CDC), and/or apoptosis.

It will be appreciated by persons skilled in the art that in one embodiment the Fc region is a naturally occurring (i.e. wildtype) human Fc region. In an alternative embodiment the Fc region is a non-naturally occurring (e.g. mutated) human Fc region.

In one embodiment the Fc region of the immunoglobulin may have modified glycosylation. For example, the Fc region may be afucosylated.

By “afucosylated”, “defucosylated” or “non-focusylated” antibodies we mean that the Fc region of the antibody does not have any fucose sugar units attached, or has a decreased content of fucose sugar units. Decreased content may be defined by the relative amount of fucose on the modified antibody compared to the fucosylated ‘wild type’ antibody, e.g. fewer fucose sugar units per immunoglobulin molecule compared to the equivalent antibody expressed in the absence of an inhibitor of mannosidase and/or in the presence of GDP-6-deoxy-D-lyxo-4-hexulose reductase.

In one embodiment, the one or more Fab fragment(s) is linked to the C-terminal end of the immunoglobulin via a linker. Accordingly, the linker may be a peptide linker linking the second light chain polypeptide to the first heavy chain polypeptide. In one embodiment, where there are two Fab fragments (and therefore two second light chain polypeptides), and wherein there are also two first heavy chain polypeptides in the immunoglobulin, each Fab fragment is attached to one of the first heavy chain polypeptides via a linker.

In one embodiment, the linker is a peptide with an amino acid sequence selected from:

| (SEQ ID NO: 2) | |
|---|---|
| | SGGGGSGGGGS, |
| (SEQ ID NO: 3) | |
| | SGGGGSGGGGSAP, |
| (SEQ ID NO: 4) | |
| | NFSQP, |
| (SEQ ID NO: 5) | |
| | KRTVA, |
| (SEQ ID NO: 6) | |
| | GGGGSGGGGGGGGS |
| | or |
| | (SG)m, |
| | where m = 1 to 7. |

### Primary Mutations

In one embodiment, the light chain region comprises a CKappa or CLambda domain, and the primary mutation is at position 114 in the CKappa or CLambda (preferably CKappa) domain (according to the EU or Kabat numbering systems) and/or the heavy chain region comprises a CH1 domain and the primary mutation is at position 187 in the CH1 domain (according to EU numbering system). In a specific embodiment, the light chain region comprises a CKappa or CLambda domain, and the primary mutation is at position 114 in the CKappa or CLambda (preferably CKappa) domain. In an alternative specific embodiment, the heavy chain region comprises a CH1 domain and the primary mutation is at position 187 in the CH1 domain. In a further embodiment, the primary mutations are at position 114 in the CKappa or CLambda (preferably CKappa) domain (according to the EU or Kabat numbering systems) and at position 187 in the CH1 domain (according to EU numbering system).

In a preferred embodiment, the primary mutation is X114A in the CKappa or CLambda (preferably CKappa) domain and/or at position X187E/D in the CH1 domain. In a specific embodiment, the primary mutation is X114A in the CKappa or CLambda (preferably CKappa) domain. In an alternative specific embodiment, the primary mutation is X187E/D in the CH1 domain. In a further embodiment, the primary mutation is X114A in the CKappa or CLambda (preferably CKappa) domain (according to the EU or Kabat numbering systems) and X187E/D in the CH1 domain (according to EU numbering system). X refers to any amino acid.

In an alternative preferred embodiment, the primary mutation is T/S114X in the CKappa or CLambda (preferably CKappa) domain and/or is T187X in the CH1 domain. In a specific embodiment, the primary mutation is T/S114X in the CKappa or CLambda (preferably CKappa) domain. In an alternative specific embodiment, the primary mutation is T187X in the CH1 domain. In a further embodiment, the primary mutation is T/S114X in the CKappa or CLambda (preferably CKappa) domain (according to the EU or Kabat numbering systems) and T187X in the CH1 domain (according to EU numbering system). X refers to any amino acid.

The use of “/” in the context of discussing mutations is to illustrate alternative possible amino acids; for example, “X187E/D” indicates that E or D can be included at position 187, as a substitute for the amino acid “X”.

In a further preferred embodiment, the primary mutation is T/S114A in the CKappa or CLambda (preferably CKappa) domain and/or is T187E/D in the CH1 domain. In a specific embodiment, the primary mutation is T/S114A in the CKappa or CLambda (preferably CKappa) domain. In an alternative specific embodiment, the primary mutation is T187E/D in the CH1 domain. In a further embodiment, the primary mutation is T/S114A in the CKappa or CLambda (preferably CKappa) domain (according to the EU or Kabat numbering systems) and T187E/D in the CH1 domain (according to EU numbering system).

In a preferred embodiment, the primary mutation at position 187 in the CHQ domain is in the “H1” chain as described herein and/or the “H2” chain as described here, preferably in the “H2” chain.

In a preferred embodiment, the primary mutation at position 114 in the CKappa or CLambda (preferably CKappa) domain is in the “H1” chain as described herein and/or the “L1” chain as described here, preferably in the “H1” chain.

TABLE A
Relevant wildtype or starting material CKappa,
CLambda, and VL J segment domain amino acid
sequences. As explained below, the boundary of
the CKappa domain is at position 108 (i.e. the
most N-terminal amino acid of the domain),
according to the EU or Kabat numbering systems,
and the boundary of the CLambda domain is at
position 107A, according to Kabat numbering
system. The adjacent amino acid is in the VL
domain, and is numbered position 127 (i.e. the
most N-terminal amino acid of the domain),
according to the IMGT numbering system. In the
table below, the last VL domain amino acid is
italicised and the first CKappa/CLambda domain
amino acid is emboldened.

| CKappa + VL or | | SEQ ID |
|---|---|---|
| CLambda + VL | Amino acid sequence | NO: |
| CKappa | RTVAAPSVFIFPPSDEQLKSGTASVVCLLN | 7 |
| | NFYPREAKVQWKVDNALQSGNSQESVTEQD | |
| | SKDSTYSLSSTLTLSKADYEKHKVYACEVT | |
| | HQGLSSPVTKSFNRGEC | |
| CLambda (1) | GQPKANPTVTLFPPSSEELQANKATLVCLI | 8 |
| | SDFYPGAVTVAWKADGSPVKAGVETTKPSK | |
| | QSNNKYAASSYLSLTPEQWKSHRSYSCQVT | |
| | HEGSTVEKTVAPTECS | |
| CLambda (2) | GQPKAAPSVTLFPPSSEELQANKATLVCLI | 9 |
| | SDFYPGAVTVAWKADSSPVKAGVETTTPSK | |
| | QSNNKYAASSYLSLTPEQWKSHRSYSCQVT | |
| | HEGSTVEKTVAPTECS | |
| CLambda (3) | GQPKAAPSVTLFPPSSEELQANKATLVCLI | 10 |
| | SDFYPGAVTVAWKADSSPVKAGVETTTPSK | |
| | QSNNKYAASSYLSLTPEQWKSHKSYSCQVT | |
| | HEGSTVEKTVAPTECS | |
| CLambda (6) | GQPKAAPSVTLFPPSSEELQANKATLVCLI | 11 |
| | SDFYPGAVKVAWKADGSPVNTGVETTTPSK | |
| | QSNNKYAASSYLSLTPEQWKSHRSYSCQVT | |
| | HEGSTVEKTVAPAECS | |
| CLambda (7) | GQPKAAPSVTLFPPSSEELQANKATLVCLV | 12 |
| | SDFNPGAVTVAWKADGSPVKVGVETTKPSK | |
| | QSNNKYAASSYLSLTPEQWKSHRSYSCRVT | |
| | HEGSTVEKTVAPAECS | |
| CKappa - relevant section | RTVAAPSVFIFPPSDEQL | 13 |
| CLambda (1) - relevant | GQPKANPTVTLFPPSSEEL | 14 |
| section | | |
| CLambda (2), (3), (6) or | GQPKAAPSVTLFPPSSEEL | 15 |
| (7) - relevant section | | |
| CKappa J1 of VL | WTFGQGTKVEIK | 16 |
| CKappa J2 of VL | YTFGQGTKLEIK | 17 |
| CKappa J3 of VL | FTFGPGTKVDIK | 18 |
| CKappa J4 of VL | LTFGGGTKVEIK | 19 |
| CKappa J5 of VL | ITFGQGTRLEIK | 20 |
| Further CKappa J of VL | TFGAGTKLEIK | 284 |
| CKappa J1 of VL- relevant | TFGQGTKVEIK | 21 |
| section | | |
| CKappa J2 of VL- relevant | TFGQGTKLEIK | 22 |
| section | | |
| CKappa J3 of VL- relevant | TFGPGTKVDIK | 23 |
| section | | |
| CKappa J4 of VL- relevant | TFGGGTKVEIK | 24 |
| section | | |
| CKappa J5 of VL- relevant | TFGQGTRLEIK | 25 |
| section | | |
| CLambda J1 of VL | YVFGTGTKVTVL | 26 |
| CLambda J2 of VL | VVFGGGTKLTVL | 27 |
| CLambda J4 of VL | FVFGGGTQLIIL | 28 |
| CLambda J5 of VL | WVFGEGTELTVL | 29 |
| CLambda J7 of VL | AVFGGGTQLTVL | 30 |
| CLambda J1 of VL - | VFGTGTKVTVL | 31 |
| relevant section | | |
| CLambda J2 of VL - relevant | VFGGGTKLTVL | 32 |
| section | | |
| CLambda J4 of VL - relevant | VFGGGTQLIIL | 33 |
| section | | |
| CLambda J5 of VL - relevant | VFGEGTELTVL | 34 |
| section | | |
| CLambda J7 of V L- relevant | VFGGGTQLTVL | 35 |
| section | | |
| CKappa + VL J1 - relevant | TFGQGTKVEIKRTVAAPSVFIFPPSDEQL | 36 |
| sections | | |
| CKappa + VL J2 - relevant | TFGQGTKLEIKRTVAAPSVFIFPPSDEQL | 37 |
| sections | | |
| CKappa + VL J3 - relevant | TFGPGTKVDIKRTVAAPSVFIFPPSDEQL | 38 |
| sections | | |
| CKappa + VL J4 - relevant | TFGGGTKVEIKRTVAAPSVFIFPPSDEQL | 39 |
| sections | | |
| CKappa + VL J5 - relevant | TFGQGTRLEIKRTVAAPSVFIFPPSDEQL | 40 |
| sections | | |
| CKappa + VL J - relevant | TFGAGTKLEIKRTVAAPSVFIFPPSDEQL | 206 |
| sections | | |
| CLambda (1) + VL J1 - | VFGTGTKVTVLGQPKANPTVTLFPPSSEEL | 41 |
| relevant sections | | |
| CLambda (2), (3), (6) or | VFGTGTKVTVLGQPKAAPSVTLFPPSSEEL | 42 |
| (7) + VL J1 - relevant | | |
| sections | | |
| CLambda (1) + VL J2 - | VFGGGTKLTVLGQPKANPTVTLFPPSSEEL | 43 |
| relevant sections | | |
| CLambda (2), (3), (6) or | VFGGGTKLTVLGQPKAAPSVTLFPPSSEEL | 44 |
| (7) + VL J2 - relevant | | |
| sections | | |
| CLambda (1) + VL J4 - | VFGGGTQLIILGQPKANPTVTLFPPSSEEL | 45 |
| relevant sections | | |
| CLambda (2), (3), (6) or | VFGGGTQLIILGQPKAAPSVTLFPPSSEEL | 46 |
| (7) + VL J4 - relevant | | |
| sections | | |
| CLambda (1) + VL J5 - | VFGEGTELTVLGQPKANPTVTLFPPSSEEL | 47 |
| relevant sections | | |
| CLambda (2), (3), (6) or | VFGEGTELTVLGQPKAAPSVTLFPPSSEEL | 48 |
| (7) + VL J5 - relevant | | |
| sections | | |
| CLambda (1) + VL J7 - | VFGGGTQLTVLGQPKANPTVTLFPPSSEEL | 49 |
| relevant sections | | |
| CLambda (2), (3), (6) or | VFGGGTQLTVLGQPKAAPSVTLFPPSSEEL | 50 |
| (7) + VL J7 - relevant | | |
| sections | | |

TABLE B
Primary mutations in CKappa/CLambda and VL, or
CKappa/CLambda, amino acid sequences relevant
to the invention. Position 114 of the CKappa/
CLambda domain is at X, according to the EU or
Kabat numbering systems. As explained for Table
A, the last VL domain amino acid is italicised
and the first CKappa/CLambda domain amino acid
is emboldened.

| CKappa, | | |
|---|---|---|
| CLambda, | | |
| CKappa + VL, | | SEQ |
| or | | ID |
| CLambda + VL | Amino acid sequence | NO: |
| CKappa + VL | TFGQGTKVEIKRTVAAPXVFIFPPSDEQL | 51 |
| CKappa + VL | TFGQGTKLEIKRTVAAPXVFIFPPSDEQL | 52 |
| CKappa + VL | TFGPGTKVDIKRTVAAPXVFIFPPSDEQL | 53 |
| CKappa + VL | TFGGGTKVEIKRTVAAPXVFIFPPSDEQL | 54 |
| CKappa + VL | TFGQGTRLEIKRTVAAPXVFIFPPSDEQL | 55 |
| CKappa + VL | TFGAGTKLEIKRTVAAPXVFIFPPSDEQL | 285 |
| CKappa + VL | EIKRTVAAPXV | 56 |
| CKappa + VL | DIKRTVAAPXV | 57 |
| CKappa | RTVAAPXV | 58 |
| CKappa or | PXV | 59 |
| CLambda | | |
| CLambda + VL | VFGTGTKVTVLGQPKANPXVTLFPPSSEEL | 60 |
| CLambda + VL | VFGTGTKVTVLGQPKAAPXVTLFPPSSEEL | 61 |
| CLambda + VL | VFGGGTKLTVLGQPKANPXVTLFPPSSEEL | 62 |
| CLambda + VL | VFGGGTKLTVLGQPKAAPXVTLFPPSSEEL | 63 |
| CLambda + VL | VFGGGTQLIILGQPKANPXVTLFPPSSEEL | 64 |
| CLambda + VL | VFGGGTQLIILGQPKAAPXVTLFPPSSEEL | 65 |
| CLambda + VL | VFGEGTELTVLGQPKANPXVTLFPPSSEEL | 66 |
| CLambda + VL | VFGEGTELTVLGQPKAAPXVTLFPPSSEEL | 67 |
| CLambda + VL | VFGGGTQLTVLGQPKANPXVTLFPPSSEEL | 68 |
| CLambda + VL | VFGGGTQLTVLGQPKAAPXVTLFPPSSEEL | 69 |
| CLambda + VL | VLGQPKANPXV | 70 |
| CLambda + VL | VLGQPKAAPXV | 71 |
| CLambda + VL | ILGQPKANPXV | 72 |
| CLambda + VL | ILGQPKAAPXV | 73 |
| CLambda | GQPKANPXV | 74 |
| CLambda | GQPKAAPXV | 289 |

X is any amino acid but preferably T or S, and it is preferably replaced by A.

In one embodiment, the primary mutation at position 114 in the CKappa or CLambda (preferably CKappa) domain is at position X in an amino acid sequence selected from Table B.

In a particular embodiment, the primary mutation at position 114 in the CKappa or CLambda (preferably CKappa) domain is at position X in a 3mer, 5mer, 7mer, 9mer, 11mer, 13mer, 15mer, 17mer, 19mer, 21mer, or 23mer of an amino acid sequence selected from Table B centered on position X; for example, a 9mer of this embodiment in sequence TFGQGTKVEIKRTVAAPXVFIFPPSDEQL (SEQ ID NO: 51) is VAAPXVFIF (SEQ ID NO: 75).

In a further embodiment, the primary mutation at position 114 in the CKappa or CLambda (preferably CKappa) domain is at position X in an amino acid sequence that comprises the sequence PXV and which has a 10% or more; 20% or more; 30% or more; 40% or more; 50% or more; 60% or more; 70% or more; 80% or more; 90% or more; or 95% or more sequence identity to an amino acid sequence selected from Table B.

In a preferred embodiment, the primary mutation at position 114 in the CKappa domain is at position X in an amino acid sequence TFGQGTKLEIKRTVAAPXVFIFPPSDEQL (SEQ ID NO: 52) or EIKRTVAAPXV (SEQ ID NO: 56) or PXV (SEQ ID NO 59).

In a further preferred embodiment, the primary mutation at position 114 in the CKappa domain is at position X in an amino acid sequence TFGAGTKLEIKRTVAAPXVFIFPPSDEQL (SEQ ID NO: 285).

In a particularly preferred embodiment, as discussed further above, the amino acid that replaces the amino acid at position 114 in any of the amino acid sequences in Table B is A.

TABLE C
Relevant wildtype CH1 amino acid sequences.

| | | SEQ ID |
|---|---|---|
| Fc Isotype | Amino acid sequence | NO: |
| IgG1 | ASTKGPSVFPLAPSSKSTSGGTAALGCLVKDYFPEPV | 290 |
| | TVSWNSGALTSGVHTFPAVLQSSGLYSLSSVVTVPS | |
| | SSLGTQTYICNVNHKPSNTKVDKKV | |
| IgG2 | ASTKGPSVFPLAPCSRSTSESTAALGCLVKDYFPEPV | 291 |
| | TVSWNSGALTSGVHTFPAVLQSSGLYSLSSVVTVPS | |
| | SNFGTQTYTCNVDHKPSNTKVDKTV | |
| IgG3 | ASTKGPSVFPLAPCSRSTSGGTAALGCLVKDYFPEPV | 292 |
| | TVSWNSGALTSGVHTFPAVLQSSGLYSLSSVVTVPS | |
| | SSLGTQTYTCNVNHKPSNTKVDKRV | |
| IgG4 | ASTKGPSVFPLAPCSRSTSESTAALGCLVKDYFPEPV | 293 |
| | TVSWNSGALTSGVHTFPAVLQSSGLYSLSSVVTVPS | |
| | SSLGTKTYTCNVDHKPSNTKVDKRV | |
| IgG1 - relevant | AVLQSSGLYSLSSVVTVPSSSLGTQTYICNV | 76 |
| sections | | |
| IgG2 - relevant | AVLQSSGLYSLSSVVTVPSSNFGTQTYTCNV | 77 |
| sections | | |
| IgG3 - relevant | AVLQSSGLYSLSSVVTVPSSSLGTQTYTCNV | 78 |
| sections | | |
| IgG4 - relevant | AVLQSSGLYSLSSVVTVPSSSLGTKTYTCNV | 79 |
| sections | | |

In one embodiment, the CH1 domain is from an Fc Isotype selected from the list comprising: IgG1; IgG2; IgG3; and IgG4.

Most preferably, the CH1 domain is from IgG1.

TABLE D
Primary mutations in CH1 amino acid sequences relevant to
the invention. Position 187 of the CH1 domain is at X,
according to the EU numbering system.

| | | SEQ ID |
|---|---|---|
| Fc Isotype | Amino acid sequence | NO: |
| IgG1 | AVLQSSGLYSLSSVVXVPSSSLGTQTYICNV | 80 |
| IgG2 | AVLQSSGLYSLSSVVXVPSSNFGTQTYTCNV | 81 |
| IgG3 | AVLQSSGLYSLSSVVXVPSSSLGTQTYTCNV | 82 |
| IgG4 | AVLQSSGLYSLSSVVXVPSSSLGTKTYTCNV | 83 |
| IgG1/IgG2/IgG3/IgG4 | GLYSLSSVVXVP | 84 |
| IgG1/IgG2/IgG3/IgG4 | VXV | 85 |

X is any amino acid but preferably T, and it is preferably replaced by E or D.

In one embodiment, the primary mutation at position 187 in the CH1 domain is at position X in the amino acid sequence selected from Table D.

In a particular embodiment, the primary mutation at position 187 in the CH1 domain is at position X in a 3mer, 5mer, 7mer, 9mer, 11mer, 13mer, 15mer, 17mer, 19mer, 21mer, 23mer, 25mer, 27mer, 29mer, or 31mer of the amino acid sequence selected from Table D; for example, a 9mer of this embodiment in sequence AVLQSSGLYSLSSVVXVPSSSLGTQTYICNV (SEQ ID NO: 80) is SSVVXVPSS (SEQ ID NO: 86).

In a further embodiment, the primary mutation at position 187 in the CH1 domain is at position X in an amino acid sequence that comprises the sequence VXV and which has a 10% or more; 20% or more; 30% or more; 40% or more; 50% or more; 60% or more; 70% or more; 80% or more; 90% or more; or 95% or more sequence identity to the amino acid sequence selected from Table D.

It will be appreciated that although the positioning of the mutation in the amino acid sequences described herein is with reference to a numerical value (for example, “the primary mutation at position 187 in the CH1 domain is at position X in the amino acid sequence AVLQSSGLYSLSSVVXVPSSSLGTQTYICNV (SEQ ID NO: 80)”), that is to allow a cross-reference between these particular embodiments and other embodiments further discussing those mutations. Therefore, it is encompassed that the mutations as described within the amino acid sequences disclosed may also be included in the context of that sequence but not at the overall position specified by the associated number. Accordingly, and as an example, by the primary mutation being at position 187 in the CH1 domain being at position X in the amino acid sequence AVLQSSGLYSLSSVVXVPSSSLGTQTYICNV (SEQ ID NO: 80) or GLYSLSSVVXVP (SEQ ID NO: 84) or VXV (SEQ ID NO: 85), it is also included that the primary mutation can be at position X in the amino acid sequence AVLQSSGLYSLSSVVXVPSSSLGTQTYICNV (SEQ ID NO: 80) or GLYSLSSVVXVP (SEQ ID NO: 84) or VXV (SEQ ID NO: 85).

### Secondary Mutations

The “secondary mutations” described herein can be referred to as the mutations of “optimised RUBY™”; in particular “mutation set 2”—including “set 2a” and/or “set 2b”.

Although bispecific polypeptides in the “RUBY™ format” are designed for the clinic and have a low immunogenicity, bispecific polypeptides in the “optimised RUBY™ format” with the “secondary mutations” have been engineered to carry an even further reduced risk of provoking immunogenic responses directed against the bispecific polypeptide itself.

It will be appreciated by the skilled person that various combinations of these secondary mutations could be used in a polypeptide (preferably bispecific polypeptide) of the invention, as well as in combination with any of the primary mutations described above. The combinations of the primary mutations and secondary mutations, used in the same polypeptide, are described below. It will also be appreciated that the variations of those mutations as described herein would also work as part of the invention. All mutations in variable domains (VH or VL) are numbered according to the IMGT numbering system, all mutations in the constant CKappa light chain domains are numbered according to the EU or Kabat numbering systems, all mutations in the constant CLambda light chain domains are numbered according to the Kabat numbering system, and all constant heavy chain mutations are numbered according to the EU numbering system.

In one embodiment, one or more secondary mutation is at one or more position 8 or fewer amino acids upstream and/or downstream from the primary mutation, in a sequence of contiguous amino acids.

In a specific embodiment, one or more secondary mutation is at one or more position 8 or fewer amino acids upstream and/or downstream from the primary mutation at position 114 in the CKappa or CLambda (preferably Ckappa) domain, in a sequence of contiguous amino acids (such as an amino acid sequence in Table B).

When discussing the positioning of amino acids upstream of position 114 in the CKappa or CLambda domain, the range might extend into the VL domain which is to the N-terminus of the CKappa or CLambda domain.

According to the EU or Kabat numbering systems, the N-terminal boundary of the CKappa domain is position 108, before which the adjacent amino acid is in the VL domain and numbered position 127, according to IMGT numbering. Accordingly, the position numbering between the CKappa domain and VL domain is as follows (CKappa domain underlined and VL emboldened—X denotes an amino acid and the number its position): X¹²³X¹²⁴X¹²⁵X¹²⁶X¹²⁷X¹⁰⁸X¹⁰⁹X¹¹⁰X¹¹¹X¹¹²X¹¹³X¹¹⁴X¹¹⁵X¹¹⁶—the boundary of the domains is as follows in the following sequence of the application: TFGQGTKVEIKRTVAAPSVFIFPPSDEOL (SEQ ID NO: 36—CKappa domain, as relevant for this sequence, underlined and VL emboldened).

According to the Kabat numbering systems, the N-terminal boundary of the CLambda domain is position 107A, before which the adjacent amino acid is in the VL domain and numbered position 127, according to IMGT numbering. Accordingly, the position numbering between the CLambda domain and VL domain is as follows (CLambda domain underlined and VL emboldened—X denotes an amino acid and the number its position): X¹²³X¹²⁴X¹²⁵X¹²⁶X¹²⁷X¹⁰⁷X¹⁰⁸X¹⁰⁹X¹¹⁰X¹¹¹X¹¹²X¹¹³X¹¹⁴X¹¹⁵X¹¹⁶—VFGTGTKVTVLGOPKANPTVTLFPPSSEEL (SEQ ID NO: 41—CLambda domain, as relevant for this sequence, underlined and VL emboldened).

Accordingly, in one embodiment the one or more secondary mutation is in a region from position 126 in the VL domain to position 122 in the CKappa domain. In an alternative embodiment, the one or more secondary mutation is in a region from position 127 in the VL domain to position 123 in the CLambda domain.

In a further specific embodiment, one or more secondary mutation is at one or more position 8 or fewer amino acids upstream and/or downstream from the primary mutation at position 187 in the CH1 domain, in a sequence of contiguous amino acids (such as an amino acid sequence selected from Table D); for example the one or more secondary mutation is in a region from position 179 to position 195 in the CH1 domain.

In one embodiment, the secondary mutation is one or more mutation at a position in an amino acid sequence selected from Table B, wherein the mutation is not at position X and wherein X is the amino acid A; and/or the secondary mutation is one or more mutation at a position in an amino acid sequence selected from Table D, wherein the mutation is not at position X and wherein X is the amino acid E or D.

In a particular embodiment, the secondary mutation is one or more mutation at a position in a 3mer, 5mer, 7mer, 9mer, 11mer, 13mer, 15mer, 17mer, 19mer, 21mer, or 23mer amino acid sequence selected from Table B centered on position X, wherein the mutation is not at position X and wherein X is the amino acid A.

In a further embodiment, the secondary mutation is one or more mutation at a position in an amino acid sequences that comprises the sequence PXV and which has a 10% or more; 20% or more; 30% or more; 40% or more; 50% or more; 60% or more; 70% or more; 80% or more; 90% or more; or 95% or more sequence identity to an amino acid sequence selected from Table B, wherein the mutation is not at position X and wherein X is the amino acid A.

In a particular embodiment, the secondary mutation is one or more mutation at a position in a 3mer, 5mer, 7mer, 9mer, 11mer, 13mer, 15mer, 17mer, 19mer, 21mer, 23mer, 25mer, 27mer, 29mer, or 31mer of the amino acid sequence selected from Table D centered on position X, wherein the mutation is not at position X and wherein X is the amino acid E or D.

In a further embodiment, the secondary mutation is one or more mutation at a position in an amino acid sequence that comprises the sequence VXV and which has a 10% or more; 20% or more; 30% or more; 40% or more; 50% or more; 60% or more; 70% or more; 80% or more; 90% or more; or 95% or more sequence identity to the amino acid sequence selected from Table D, wherein the mutation is not at position X and wherein X is the amino acid E or D.

In one embodiment, the secondary mutation is at one or more of positions 1, 4 and 6 of the amino acid sequence XKRXVXAPA (SEQ ID NO: 87) in a CKappa and VL domain wherein X is any amino acid, wherein position 9 is the primary mutation, and wherein XK is part of the VL domain and RXVXAPA is part of the CKappa domain. In a preferred embodiment, the secondary mutation is at position 4 and/or 6 of the amino acid sequence IKRXVXAPA (SEQ ID NO: 88) in a CKappa and VL domain wherein X is any amino acid, wherein position 9 is the primary mutation, and wherein IK is part of the VL domain and RXVXAPA is part of the CKappa domain. Positions 1, 4 and 6 in this embodiment correspond to positions 126 of the VL domain, and position 109 and 111 in the CKappa domain of embodiments herein. Longer versions of the amino acid sequences of this embodiment are described in Table B, and those longer sequences including the secondary mutations described in this embodiment would be appreciated by the skilled person.

In an alternative embodiment, the secondary mutation is at position 4 and/or 6 of the amino acid sequence LGQXKXNPA (SEQ ID NO: 89) in a CLambda and VL domain wherein X is any amino acid, wherein position 9 is the primary mutation, and wherein L is part of the VL domain and GQXKXNPA is part of the CLambda domain. In a further alternative embodiment, the secondary mutation is at position 4 and/or 6 of the amino acid sequence LGQXKXAPA (SEQ ID NO: 90) in a CLambda and VL domain wherein X is any amino acid, wherein position 9 is the primary mutation, and wherein L is part of the VL domain and GQXKXAPA is part of the CLambda domain. Position 4 and 6 in this embodiment correspond to position 109 and 111 in the CLambda domain of embodiments herein. Longer versions of the amino acid sequences of this embodiment are described in Table A and B, and those longer sequences including the secondary mutations described in this embodiment would be appreciated by the skilled person.

In one embodiment, the secondary mutation is at one or more of positions 1, 4, 6 and 9 of the amino acid sequence XSLXSXVEX (SEQ ID NO: 91) in a CH1 domain, wherein X is any amino acid and wherein position 8 is the primary mutation; XSLXSXVDX (SEQ ID NO: 92) in a CH1 domain, wherein X is any amino acid and wherein position 8 is the primary mutation; YSLSSVVEV (SEQ ID NO: 93) in a CH1 domain, wherein position 8 is the primary mutation; or YSLSSVVEV (SEQ ID NO: 93) in a CH1 domain, wherein position 8 is the primary mutation; preferably positions 1 and 4, more preferably position 4. Positions 1, 4, 6 and 9 in this embodiment correspond to positions 180, 183, 185 and 188 in the embodiments below. Longer versions of the amino acid sequences of this embodiment are described in Table C and D, and those longer sequences including the secondary mutations described in this embodiment would be appreciated by the skilled person.

As discussed further below and in the Examples, the inventors have surprisingly identified that the relative position of the secondary mutation(s) to the primary mutation can be relevant for reducing the immunogenicity of the primary mutation. That is because the relative positions of the primary and secondary mutations can be important to changing the binding characteristics of the polypeptide for receptors that cause immunogenicity, such as MHC Class II.

In one embodiment, the heavy chain region comprises a CH1 domain and the secondary mutation is at position 180, 183, and/or 188 in the CH1 domain, preferably 180 or 183, more preferably 183 (according to the EU numbering system).

In one embodiment, the light chain region comprises a CKappa or Clambda (preferably CKappa) domain and the secondary mutation is at position 111 and/or 109 in the CKappa or Clambda (preferably CKappa) domain.

In one embodiment, the light chain region comprises a VL domain and the secondary mutation is at position 126 in the VL domain.

In a particular embodiment, the secondary mutations are one or more of the following positions in the CKappa or CLambda (preferably CKappa) domain: 111 and 109 (according to the EU or Kabat numbering system); and 126 position in the VL domain (according to the IMGT numbering system).

In one embodiment of the invention the secondary mutations are at positions selected from the group consisting of:
(a) one or more of the following positions in the CH1 domain: 180; 183; and 188, preferably 180 (according to the EU numbering system); and/or
(b) one or more of the following positions in the CKappa or CLambda (preferably CKappa) domain: 111 and 109 (according to the EU or Kabat numbering systems); and/or
(c) the 126 position in the VL domain (according to the IMGT numbering system).

In a particular embodiment, the secondary mutations are at two or more of the following positions: 180 in the CH1 domain; 183 in the CH1 domain; 188 in the CH1 domain; 111 in the CKappa or CLambda (preferably CKappa) domain; 109 in the CKappa or CLambda (preferably CKappa) domain; and 126 in the VL domain, for example: three or more; four or more; five or more; or all six of those positions.

In one embodiment of the invention the secondary mutations are at positions selected from the group consisting of:
(a) one or more of the following positions in the CH1 domain: Y180; S183; and V188, preferably Y180 (according to the EU numbering system); and/or
(b) one or more of the following positions in the CKappa domain: A111 and T109 or one or more of the following positions in the CLambda domain: A111 and P109 (according to the EU or Kabat numbering systems); and/or
(c) the 1126 position in the VL domain (according to the IMGT numbering system).

In a particular embodiment, the secondary mutations are one or more of the following positions in the CH1 domain: Y180; S183; and V188, preferably Y180 or S183, more preferably S183 (according to the EU numbering system).

In a particular embodiment, the secondary mutations are one or more of the following positions in the CKappa domain: A111 and T109 (according to the EU or Kabat numbering system); and/or the 1126 position in the VL (according to the IMGT numbering system). In a particular alternative embodiment, the secondary mutations are one or more of the following positions in the CLambda domain: A111 and P109 (according to the Kabat numbering system).

In one embodiment of the invention the secondary mutations are selected from the group consisting of:
(a) one or more of the following mutations in the CH1 domain: Y180X; S183X; and V188X; preferably, Y180X (according to the EU numbering system); and/or
(b) one or more of the following mutations in the CKappa domain: A111X; and T109X or one or more of the following positions in the CLambda domain: A111X and P109X (according to the EU or Kabat numbering systems); and/or
(c) I126X in the VL domain (according to the IMGT numbering system).
*X refers to any amino acid

In a particular embodiment, the secondary mutation is one or more of the following mutations in the CH1 domain: Y180X; S183X; and V188X; preferably, Y180X or S183X, more preferably S183X (according to the EU numbering system).
*X refers to any amino acid

In a particular embodiment, the secondary mutation is one or more of the following mutations in the CKappa domain: A111X; and T109X (according to the EU or Kabat numbering systems); and/or the mutation is I126X in the VL domain (according to the IMGT numbering system). In a particular alternative embodiment, the secondary mutations are one or more of the following positions in the CLambda domain: A111X and P109X (according to the EU numbering system).
*X refers to any amino acid

In one embodiment of the invention the secondary mutations are selected from the group consisting of:
(a) one or more of the following mutations in the CH1 domain: X180A/G/I/N/S/T/V/W; X183N/T; and X188G; preferably, X180T (according to the EU numbering system); and/or
(b) one or more of the following mutations in the CKappa or CLambda (preferably CKappa) domain: X111R/T/W/V and X109P, preferably X111V and X109P (according to the EU or Kabat numbering systems); and/or
(c) X126A/G/H/N/P/Q/S/T in the VL (according to the IMGT numbering system).
*X refers to any amino acid

The use of “/” in the context of discussing mutations is to illustrate alternative possible amino acids; for example, “X180A/G/I/N/S/T/V/W” indicates that A or G or I or or S or T or V or W can be included at position 180, as a substitute for the amino acid “X”.

In a particular embodiment, the secondary mutation is one or more of the following mutations in the CH1 domain: X180A/G/I/N/S/T/V/W; X183N/T; and X188G; preferably, X180T or X183N/T, more preferably X183N/T (according to the EU numbering system).
*X refers to any amino acid

In a particular embodiment, the secondary mutation is one or more of the following mutations in the CKappa or CLambda (preferably CKappa) domain: X111R/T/W/V and X109P, preferably X111V and X109P (according to the EU or Kabat numbering systems); and/or the mutation is X126A/G/H/N/P/Q/S/T in the VL (according to the IMGT numbering system).
*X refers to any amino acid

For example, the secondary mutations may be selected from the group consisting of:
(a) one or more of the following mutations in the CH1 domain: Y180A; Y180G; Y180I; Y180N; Y180S; Y180T; Y180V; or Y180W, and/or S183N or S183T, and/or V188G, preferably Y180T (according to the EU numbering system); and/or
(b) one or more of the following mutations in the CKappa or CLambda (preferably CKappa) domain A111R; A111T; A111W; or A111V, and/or T109P, preferably T109P and A111V (according to the EU or Kabat numbering systems); and/or
(c) one of the following mutations in the VL domain: I126A; I126G; I126H; I126N; I126P; I126Q; I126S; or I126T (according to the IMGT numbering system).

In a particular example, the secondary mutations are one or more of the following mutations in the CH1 domain: Y180A; Y180G; Y180I; Y180N; Y180S; Y180T; Y180V; or Y180W, and/or S183N or S183T, and/or V188G, preferably Y180T; S183N; and/or S183T, more preferably S183N and/or S183T (according to the EU numbering system).

In a particular example, the secondary mutations are one or more of the following mutations in the CKappa or CLambda (preferably CKappa) domain: A111R; A111T; A111W; or A111V, and/or T109P, preferably T109P and/or A111V (according to the EU or Kabat numbering systems); and/or one of the following mutations in the VL domain: I126A; I126G; I126H; I126N; I126P; I126Q; I126S; or I126T (according to the IMGT numbering system).

In one embodiment, the mutation is not A111R.

In a preferred embodiment, in the bispecific antibody described herein the secondary mutation at position 180 in the CH1 domain is in the “H1” chain as described herein and/or the “H2” chain as described here, preferably in the “H2” chain.

In a preferred embodiment, in the bispecific antibody described herein the secondary mutation at position 183 in the CH1 domain is in the “H1” chain as described herein and/or the “H2” chain as described here, preferably in the “H2” chain.

In a preferred embodiment, in the bispecific antibody described herein the secondary mutation at position 188 in the CH1 domain is in the “H1” chain as described herein and/or the “H2” chain as described here, preferably in the “H2” chain.

In a preferred embodiment, in the bispecific antibody described herein the secondary mutation at position 109 in the CKappa or CLambda (preferably CKappa) domain is in the “H1” chain as described herein and/or the “L1” chain as described here, preferably in the “H1” chain.

In a preferred embodiment, in the bispecific antibody described herein the secondary mutation at position 111 in the CKappa or CLambda (preferably CKappa) domain is in the “H1” chain as described herein and/or the “L1” chain as described here, preferably in the “H1” chain.

In a preferred embodiment, in the bispecific antibody described herein the secondary mutation at position 126 in the VL domain, is in the “L1” chain as described herein and/or the “L1” chain as described here, preferably in the “H1” chain.

In a particularly preferred embodiment, the secondary mutations are secondary mutation pairs; for example, a mutation at position 126 of the VL domain and a mutation at position 180 of the CH1 domain.

In a preferred embodiment, the secondary mutation pairs are at positions selected from the list consisting of:
position 126 of the VL domain and position 180 of the CH1 domain;
position 126 of the VL domain and position 183 of the CH1 domain;
position 126 of the VL domain and position 188 of the CH1 domain;
position 109 of the CKappa domain and position 180 of the CH1 domain;
position 109 of the CKappa domain and position 183 of the CH1 domain;
position 109 of the CKappa domain and position 188 of the CH1 domain;
position 111 of the CKappa domain and position 180 of the CH1 domain;
position 111 of the CKappa domain and position 183 of the CH1 domain; and
position 111 of the CKappa domain and position 188 of the CH1 domain, preferably position 109 of the CKappa domain and position 183 of the CH1 domain.

In a particularly preferred embodiment, the secondary mutation pairs are selected from the list consisting of:
mutation I126A in the VL domain and mutation Y180I in the CH1 domain;
mutation I126A in the VL domain and mutation Y180W in the CH1 domain;
mutation I126A in the VL domain and mutation S183T in the CH1 domain;
mutation I126A in the VL domain and mutation V188G in the CH1 domain;
mutation I126G in the VL domain and mutation Y180I in the CH1 domain;
mutation I126G in the VL domain and mutation Y180W in the CH1 domain;
mutation I126G in the VL domain and mutation S183T in the CH1 domain;
mutation I126G in the VL domain and mutation V188G in the CH1 domain;
mutation I126S in the VL domain and mutation Y180I in the CH1 domain;
mutation I126S in the VL domain and mutation Y180W in the CH1 domain;
mutation I126S in the VL domain and mutation S183T in the CH1 domain;
mutation I126S in the VL domain and mutation V188G in the CH1 domain;
mutation T109P in the CKappa domain and mutation Y180I in the CH1 domain;
mutation T109P in the CKappa domain and mutation Y180W in the CH1 domain;
mutation T109P in the CKappa domain and mutation S183T in the CH1 domain;
mutation T109P in the CKappa domain and mutation V188G in the CH1 domain;
mutation A111V in the CKappa domain and mutation Y180I in the CH1 domain;
mutation A111V in the CKappa domain and mutation Y180W in the CH1 domain;
mutation A111V in the CKappa domain and mutation S183T in the CH1 domain; and
mutation A111V in the CKappa domain and mutation V188G in the CH1 domain, preferably:
mutation I126S in the VL domain and mutation S183T in the CH1 domain
mutation I126S in the VL domain and mutation V188G in the CH1 domain
mutation T109P in the CKappa domain and mutation S183T in the CH1 domain;
mutation T109P in the CKappa domain and mutation V188G in the CH1 domain;
mutation A111V in the CKappa domain and mutation S183T in the CH1 domain; and
mutation A111V in the CKappa domain and mutation V188G in the CH1 domain; most preferably mutation T109P in the CKappa domain and mutation S183T in the CH1 domain.

TABLE E
Secondary mutations in CKappa and VL, or CKappa/CLambda, amino
acid sequences relevant to the invention. Position 109 of the CKappa/CLambda
domain is at X¹⁰⁹ and position 111 of the CKappa/CLambda domain is at X¹¹¹ (according
to the EU or Kabat numbering systems), and position 126 of the VL domain is at X¹²⁶
(according to the IMGT numbering system). As explained for Table A, the last VL
domain amino acid is italicised and the first CKappa/CLambda domain amino acid is
emboldened.

| CKappa + VL, or | | SEQ ID |
|---|---|---|
| CLambda + VL | Amino acid sequence | NO: |
| CKappa + VL | TFGQGTKVX¹²⁶IKRX¹⁰⁹VX¹¹¹APAVFIFPPSDEQL | 94 |
| CKappa + VL | TFGQGTKLX¹²⁶IKRX¹⁰⁹VX¹¹¹APAVFIFPPSDEQL | 95 |
| CKappa + VL | TFGPGTKVX¹²⁶IKRX¹⁰⁹VX¹¹¹APAVFIFPPSDEQL | 96 |
| CKappa + VL | TFGGGTKVX¹²⁶IKRX¹⁰⁹VX¹¹¹APAVFIFPPSDEQL | 97 |
| CKappa + VL | TFGQGTRLX¹²⁶IKRX¹⁰⁹VX¹¹¹APAVFIFPPSDEQL | 98 |
| CKappa + VL | TFGAGTKLX¹²⁶IKRX¹⁰⁹VX¹¹¹APAVFIFPPSDEQL | 286 |
| CKappa + VL | TFGQGTKVX¹²⁶IKRX¹⁰⁹VX¹¹¹APSVFIFPPSDEQL | 99 |
| CKappa + VL | TFGQGTKLX¹²⁶IKRX¹⁰⁹VX¹¹¹APSVFIFPPSDEQL | 100 |
| CKappa + VL | TFGPGTKVX¹²⁶IKRX¹⁰⁹VX¹¹¹APSVFIFPPSDEQL | 101 |
| CKappa + VL | TFGGGTKVX¹²⁶IKRX¹⁰⁹VX¹¹¹APSVFIFPPSDEQL | 102 |
| CKappa + VL | TFGQGTRLX¹²⁶IKRX¹⁰⁹VX¹¹¹APSVFIFPPSDEQL | 103 |
| CKappa + VL | TFGAGTKLX¹²⁶IKRX¹⁰⁹VX¹¹¹APSVFIFPPSDEQL | 287 |
| CKappa + VL | TFGQGTKVX¹²⁶IKRX¹⁰⁹VX¹¹¹A | 104 |
| CKappa + VL | TFGQGTKLX¹²⁶IKRX¹⁰⁹VX¹¹¹A | 105 |
| CKappa + VL | TFGPGTKVX¹²⁶IKRX¹⁰⁹VX¹¹¹A | 106 |
| CKappa + VL | TFGGGTKVX¹²⁶IKRX¹⁰⁹VX¹¹¹A | 107 |
| CKappa + VL | TFGQGTRLX¹²⁶IKRX¹⁰⁹VX¹¹¹A | 108 |
| CKappa + VL | TFGAGTKLX¹²⁶IKRX¹⁰⁹VX¹¹¹A | 294 |
| CKappa + VL | VX¹²⁶IKRX¹⁰⁹VX¹¹¹APAV | 109 |
| CKappa + VL | LX¹²⁶IKRX¹⁰⁹VX¹¹¹APAV | 110 |
| CKappa + VL | VX¹²⁶IKRX¹⁰⁹VX¹¹¹A | 111 |
| CKappa + VL | LX¹²⁶IKRX¹⁰⁹VX¹¹¹A | 112 |
| CKappa | RX¹⁰⁹VX¹¹¹APAV | 113 |
| CKappa | RX¹⁰⁹VX¹¹¹A | 114 |
| CKappa | VX¹¹¹APAV | 115 |
| CLambda + VL | VFGTGTKVTVLGQX¹⁰⁹KX¹¹¹NPAVTLFPPSSEEL | 116 |
| CLambda + VL | VFGTGTKVTVLGQX¹⁰⁹KX¹¹¹APAVTLFPPSSEEL | 117 |
| CLambda + VL | VFGGGTKLTVLGQX¹⁰⁹KX¹¹¹NPAVTLFPPSSEEL | 118 |
| CLambda + VL | VFGGGTKLTVLGQX¹⁰⁹KX¹¹¹APAVTLFPPSSEEL | 119 |
| CLambda + VL | VFGGGTQLIILGQX¹⁰⁹KX¹¹¹NPAVTLFPPSSEEL | 120 |
| CLambda + VL | VFGGGTQLIILGQX¹⁰⁹KX¹¹¹APAVTLFPPSSEEL | 121 |
| CLambda + VL | VFGEGTELTVLGQX¹⁰⁹KX¹¹¹NPAVTLFPPSSEEL | 122 |
| CLambda + VL | VFGEGTELTVLGQX¹⁰⁹KX¹¹¹APAVTLFPPSSEEL | 123 |
| CLambda + VL | VFGGGTQLTVLGQX¹⁰⁹KX¹¹¹NPAVTLFPPSSEEL | 124 |
| CLambda + VL | VFGGGTQLTVLGQX¹⁰⁹KX¹¹¹APAVTLFPPSSEEL | 125 |
| CLambda + VL | VFGTGTKVTVLGQX¹⁰⁹KX¹¹¹NPTVTLFPPSSEEL | 126 |
| CLambda + VL | VFGTGTKVTVLGQX¹⁰⁹KX¹¹¹APSVTLFPPSSEEL | 127 |
| CLambda + VL | VFGGGTKLTVLGQX¹⁰⁹KX¹¹¹NPTVTLFPPSSEEL | 128 |
| CLambda + VL | VFGGGTKLTVLGQX¹⁰⁹KX¹¹¹APSVTLFPPSSEEL | 129 |
| CLambda + VL | VFGGGTQLIILGQX¹⁰⁹KX¹¹¹NPTVTLFPPSSEEL | 130 |
| CLambda + VL | VFGGGTQLIILGQX¹⁰⁹KX¹¹¹APSVTLFPPSSEEL | 131 |
| CLambda + VL | VFGEGTELTVLGQX¹⁰⁹KX¹¹¹NPTVTLFPPSSEEL | 132 |
| CLambda + VL | VFGEGTELTVLGQX¹⁰⁹KX¹¹¹APSVTLFPPSSEEL | 133 |
| CLambda + VL | VFGGGTQLTVLGQX¹⁰⁹KX¹¹¹NPTVTLFPPSSEEL | 134 |
| CLambda + VL | VFGGGTQLTVLGQX¹⁰⁹KX¹¹¹APSVTLFPPSSEEL | 135 |
| CLambda + VL | VFGTGTKVTVLGQX¹⁰⁹KX¹¹¹N | 136 |
| CLambda + VL | VFGTGTKVTVLGQX¹⁰⁹KX¹¹¹A | 137 |
| CLambda + VL | VFGGGTKLTVLGQX¹⁰⁹KX¹¹¹N | 138 |
| CLambda + VL | VFGGGTKLTVLGQX¹⁰⁹KX¹¹¹A | 139 |
| CLambda + VL | VFGGGTQLIILGQX¹⁰⁹KX¹¹¹N | 140 |
| CLambda + VL | VFGGGTQLIILGQX¹⁰⁹KX¹¹¹A | 141 |
| CLambda + VL | VFGEGTELTVLGQX¹⁰⁹KX¹¹¹N | 142 |
| CLambda + VL | VFGEGTELTVLGQX¹⁰⁹KX¹¹¹A | 143 |
| CLambda + VL | VFGGGTQLTVLGQX¹⁰⁹KX¹¹¹N | 144 |
| CLambda + VL | VFGGGTQLTVLGQX¹⁰⁹KX¹¹¹A | 145 |
| CLambda | GQX¹⁰⁹KX¹¹¹NPAV | 146 |
| CLambda | GQX¹⁰⁹KX¹¹¹APAV | 147 |
| CLambda | GQX¹⁰⁹KX¹¹¹N | 148 |
| CLambda | GQX¹⁰⁹KX¹¹¹A | 149 |
| CLambda | KX¹¹¹NPAV | 150 |
| CLambda | KX¹¹¹APAV | 151 |

X¹⁰⁹ is any amino acid but preferably T, and it is preferably replaced by P.
X¹¹¹ is any amino acid but preferably A, and it is preferably replaced by R or T or W or V.
X¹²⁶ is any amino acid but preferably I, and it is preferably replaced by A or G or H or N or P or Q or S or T.

In one embodiment, the secondary mutation at position 109 in the CKappa or CLambda domain is at position X¹⁰⁹ and/or the secondary mutation at position 111 in the CKappa or CLambda domain is at position X¹¹¹ and/or the secondary mutation at position 126 in the VL domain is at position X¹²⁶ in the amino acid sequence selected from Table E. In a particular embodiment, the secondary mutation at position 109 in the CKappa domain is at position X¹⁰⁹ and the secondary mutation at position 111 in the CKappa domain is at position X¹¹¹ and the secondary mutation at position 126 in the VL domain is at position X¹²⁶ in the amino acid sequence selected from Table E. In a further embodiment, the secondary mutation at position 109 in the CKappa or CLambda domain is at position X¹⁰⁹ and the secondary mutation at position 111 in the CKappa or CLambda domain is at position X¹¹¹ in the amino acid sequence selected from Table E. In a further embodiment, the secondary mutation at position 109 in the CKappa domain is at position X¹⁰⁹ and the secondary mutation at position 126 in the VL domain is at position X¹²⁶ in the amino acid sequence selected from Table E. In a further embodiment, the secondary mutation at position 111 in the CKappa domain is at position X¹¹¹ and the secondary mutation at position 126 in the VL domain is at position X¹²⁶ in the amino acid sequence selected from Table E.

In a particular embodiment, the secondary mutation at position 109 in the CKappa or CLambda domain is at position X¹⁰⁹ in the amino acid sequence selected from Table E. In an alternative particular embodiment, the secondary mutation at position 111 in the CKappa or CLambda domain is at position X¹¹¹. In an alternative particular embodiment, the secondary mutation at position 126 in the VL domain is at position X¹²⁶ in the amino acid sequence selected from Table E. As will be appreciated, where a mutation is not present at one or two of positions X¹⁰⁹, X¹¹¹ and X¹²⁶ the amino acid in the same position from the corresponding wildtype amino acid sequence in Table A will be included.

In one embodiment, the amino acid sequence comprises a sequence comprising X¹⁰⁹ and/or X¹¹¹ and/or X¹²⁶ which has a 10% or more; 20% or more; 30% or more; 40% or more; 50% or more; 60% or more; 70% or more; 80% or more; 90% or more; or 95% or more sequence identity to an amino acid sequence selected from Table E.

TABLE F
Secondary mutations in CH1 amino acid sequences relevant to the
invention. Position 180 is at X¹⁸⁰ and position 183 is at X¹⁸³
and position 188 is at X¹⁸⁸, according to the EU numbering system.

| Fc Isotype | Amino acid sequence | SEQ ID NO: |
|---|---|---|
| IgG1 | AVLQSSGLX¹⁸⁰SLX¹⁸³SVVTX¹⁸⁸PSSSLGTQTYICNV | 152 |
| IgG2 | AVLQSSGLX¹⁸⁰SLX¹⁸³SVVTX¹⁸⁸PSSNFGTQTYTCNV | 153 |
| IgG3 | AVLQSSGLX¹⁸⁰SLX¹⁸³SVVTX¹⁸⁸PSSSLGTQTYTCNV | 154 |
| IgG4 | AVLQSSGLX¹⁸⁰SLX¹⁸³SVVTX¹⁸⁸PSSSLGTKTYTCNV | 155 |
| IgG1 | AVLQSSGLX¹⁸⁰SLX¹⁸³SVVEX¹⁸⁸PSSSLGTQTYICNV | 156 |
| IgG2 | AVLQSSGLX¹⁸⁰SLX¹⁸³SVVEX¹⁸⁸PSSNFGTQTYTCNV | 157 |
| IgG3 | AVLQSSGLX¹⁸⁰SLX¹⁸³SVVEX¹⁸⁸PSSSLGTQTYTCNV | 158 |
| IgG4 | AVLQSSGLX¹⁸⁰SLX¹⁸³SVVEX¹⁸⁸PSSSLGTKTYTCNV | 159 |
| IgG1 | AVLQSSGLX¹⁸⁰SLX¹⁸³SVVDX¹⁸⁸PSSSLGTQTYICNV | 160 |
| IgG2 | AVLQSSGLX¹⁸⁰SLX¹⁸³SVVDX¹⁸⁸PSSNFGTQTYTCNV | 161 |
| IgG3 | AVLQSSGLX¹⁸⁰SLX¹⁸³SVVDX¹⁸⁸PSSSLGTQTYTCNV | 162 |
| IgG4 | AVLQSSGLX¹⁸⁰SLX¹⁸³SVVDX¹⁸⁸PSSSLGTKTYTCNV | 163 |
| IgG1/IgG2/ | LX¹⁸⁰SLX¹⁸³SVVTX¹⁸⁸P | 164 |
| IgG3/IgG4 | | |
| IgG1/IgG2/ | LX¹⁸⁰SLX¹⁸³SVVEX¹⁸⁸P | 165 |
| IgG3/IgG4 | | |
| IgG1/IgG2/ | LX¹⁸⁰SLX¹⁸³SVVDX¹⁸⁸P | 166 |

IgG3/IgG4
X¹⁸⁰ is any amino acid but preferably Y, and it is preferably replaced by A or G or I or N or S or Tor V or W
X¹⁸³ is any amino acid but preferably S, and it is preferably replaced by N or T
X¹⁸⁸ is any amino acid but preferably V, and it is preferably replaced by G

In one embodiment, the secondary mutations in the CH1 domain at position 180 is at position X¹⁸⁰ and/or position 183 is at position X¹⁸³ and/or position 188 is at position X¹⁸⁸ in the amino acid sequence selected from Table F. In one embodiment, the secondary mutations in the CH1 domain at position 180 is at position X¹⁸⁰ and position 183 is at position X¹⁸³ and position 188 is at position X¹⁸⁸ in the amino acid sequence selected from Table F. In one embodiment, the secondary mutations in the CH1 domain at position 180 is at position X¹⁸⁰ and position 183 is at position X¹⁸³ in the amino acid sequence selected from Table F. In one embodiment, the secondary mutations in the CH1 domain at position 180 is at position X¹⁸⁰ and position 188 is at position X¹⁸⁸ in the amino acid sequence selected from Table F. In one embodiment, the secondary mutations in the CH1 domain at position 183 is at position X¹⁸³ and position 188 is at position X¹⁸⁸ in the amino acid sequence selected from Table F.

In a particular embodiment, the secondary mutation in the CH1 domain at position 180 is at position X¹⁸⁰ in the amino acid sequence selected from Table F. In an alternative particular embodiment, the secondary mutation in the CH1 domain at position 183 is at position X¹⁸³ in the amino acid sequence selected from Table F. In an alternative particular embodiment, the secondary mutation in the CH1 domain at position 188 is at position X¹⁸⁸ in the amino acid sequence selected from Table F. As will be appreciated, where a mutation is not present at one or two of positions X¹⁸⁰, X¹⁸³ and X¹⁸⁸ the amino acid in the same position from the corresponding wildtype amino acid sequence in Table C will be included.

In one embodiment, the amino acid sequence comprises a sequence comprising X¹⁸⁰ and/or X¹⁸³ and/or X¹⁸⁸ which has a 10% or more; 20% or more; 30% or more; 40% or more; 50% or more; 60% or more; 70% or more; 80% or more; 90% or more; or 95% or more sequence identity to an amino acid sequence selected from Table F.

As discussed above, it will be appreciated that although the positioning of the mutation in the amino acid sequences described herein is with reference to a numerical value, that is to allow a cross-reference between these particular embodiments and other embodiments further discussing those mutations. Therefore, it is encompassed that the mutations as described within the amino acid sequences disclosed may also be included in the context of that sequence but not at the overall position specified by the associated number. Accordingly, and as an example, by the secondary mutation in the CH1 domain at position 180 being at position X¹⁸⁰ and/or position 183 being at position X¹⁸³ and/or position 188 being at position X¹⁸⁸ in the amino acid sequence AVLQSSGLX¹⁸⁰SLX¹⁸³SVVEX¹⁸⁸PSSSLGTQTYICNV (SEQ ID NO: 156), it is also included that the secondary mutation can be one or more of positions X in the amino acid sequence AVLQSSGLXSLXSVVEXPSSSLGTQTYICNV (SEQ ID NO: 156).

As a reference, Mutation set 2—any individual and/or any combination of the mutations listed in set 2a and set 2b. Set 2a—mutations in the CH1: Y180A, Y180G, Y180I, Y180N, Y180S, Y180T, Y180V, or Y180W, and/or S183N or S183T, and/or V188G; preferably, Y180T and/or S183T. Set 2b—mutations in the CKappa domain: A111R, A111T, A111W or A111V, and/or T109P; preferably: T109P and/or A111V; and/or mutations in the variable domain light (VL): I126A, I126G, I126H, I126N, I126P, I126Q, I126S, or I126T.

In a further embodiment, the secondary mutations may further comprise position 185 in the CH1 domain (according to the EU numbering system).

In a further embodiment, the secondary mutations may further comprise V185X in the CH1 domain (according to the EU numbering system) *X refers to any amino acid.

### Improved Purity Mutations

The “improved purity mutations” described herein can also be referred to as the mutations of “optimised RUBY™”; in particular “mutation set 1”.

Although bispecific polypeptides in the “RUBY™ format” can be reproducibly produced with an excellent level of purity, bispecific polypeptides in the “optimised RUBY™ format” with the “improved purity mutations” can be reproducibly produced at an even higher level of purity.

It will be appreciated by the skilled person that the improved purity mutations could be used in a polypeptide (preferably bispecific polypeptide) of the invention, as well as in combination with any of the primary and/or secondary mutations described above.

In one embodiment of the invention the improved purity mutation is at position 65 in the VH domain (according to the IMGT numbering system).

In a preferred embodiment, the improved purity mutation is X65E/A/I in the VH domain. In an alternative preferred embodiment, the improved purity mutation is T65X in the VH domain.

In a further preferred embodiment, the improved purity mutation in the VH domain is T65E or T65A or T65I.

In a preferred embodiment, in the bispecific antibody described herein the improved purity at position 65 in the VH domain is in the “H1” chain as described herein and/or the “H2” chain as described here, preferably in the “H2” chain.

TABLE G
Improved purity mutation in the exemplary IGHV3 germline gene
family member IGHV3-23*01. Position 65 of the VH domain is at X⁶⁵,
according to the IMGT numbering system. CDR2 is underlined.
X^{cn} is an amino acid of CDR2 other than X⁶⁵, wherein X^{c} is any
amino acid and n is a number between 3 and 12, preferably
between 5 and 10, most preferably 7 - for example for CDR2
wherein n = 7, the sequence is X^{c}X^{c}X^{c}X^{c}X^{c}X^{c}X^{c}X⁶⁵.

| Amino acid sequence | SEQ ID NO: |
|---|---|
| EVQLLESGGGLVQPGGSLRLSCAASGFTFSSYAMSWVRQAPGKGLEWVS | 167 |
| AISGSGGSX⁶⁵YYADSVKGRFTISRDNSKNTLYLQMNSLRAEDTAVYYCAK | |
| PGKGLEWVSAISGSGGSX⁶⁵YYADSVKGRF | 168 |
| WVSAISGSGGSX⁶⁵YYADS | 169 |
| AISGSGGSX⁶⁵Y | 170 |
| ISGSGGSX⁶⁵ | 171 |
| EVQLLESGGGLVQPGGSLRLSCAASGFTFSSYAMSWVRQAPGKGLEWVS | 172 |
| AX^{cn}X⁶⁵YYADSVKGRFTISRDNSKNTLYLQMNSLRAEDTAVYYCAK | |
| PGKGLEWVSAX^{cn}X⁶⁵YYADSVKGRF | 173 |
| WVSAX^{cn}X⁶⁵YYADS | 173 |
| AX^{cn}X⁶⁵Y | 175 |

X⁶⁵ is any amino acid but preferably T, and it is preferably replaced by E or A or I.

In one embodiment, the improved purity mutation at position 65 in the VH domain is at position X⁶⁵ in an amino acid sequence selected from Table G.

In one embodiment, the amino acid sequence comprises a sequence comprising X⁶⁵ which has a 10% or more; 20% or more; 30% or more; 40% or more; 50% or more; 60% or more; 70% or more; 80% or more; 90% or more; or 95% or more sequence identity to an amino acid sequence selected from Table G.

In a third aspect, the invention provides a bispecific antibody comprising:
(a) an immunoglobulin molecule having specificity for a first antigen, the immunoglobulin molecule comprising two first heavy chain regions and two first light chain regions; and
(b) two Fab fragment having specificity for a second antigen, the Fab fragment comprising a second heavy chain region and a second light chain region;
wherein the second light chain region is fused to the C-terminus of the first heavy chain region;
and wherein the second heavy chain region comprises a VH domain, wherein the VH domain comprises a protein A binding site, and wherein the VH domain comprises a mutation that reduces or avoids the binding of protein A to the protein A binding site.

In a preferred embodiment of the third aspect invention, the polypeptide can comprise any of the mutations described in the second aspect of the invention, as well as any other features described therein.

A particular embodiment of the third aspect of the invention provides a bispecific antibody comprising:
(a) an immunoglobulin molecule having specificity for a first antigen, the immunoglobulin molecule comprising two first heavy chain regions and two first light chain regions; and
(b) two Fab fragment having specificity for a second antigen, the Fab fragment comprising a second heavy chain region and a second light chain region;
wherein the second light chain region is fused via a linker to the C-terminus of the first heavy chain region;
wherein the bispecific antibody comprises: two H1 chains, each comprising a first heavy chain region of the immunoglobulin, a linker and a second light chain region of a Fab; two L1 chains, each comprising a first light chain region of the immunoglobulin; and two H2 chains comprising a second heavy chain region of a Fab;
and wherein the two second heavy chain regions (i.e. H2) comprises a VH domain comprising a mutation at position 65 (according to the IMGT numbering system).

As a reference, mutation set 1—Mutations in the variable domain heavy (VH): T65E, T65A, T65I.

### Particularly Preferred Combinations of the Primary Mutations and Mutations of “Optimised RUBY™”

As discussed above, any of the primary mutations disclosed herein can be combined with the secondary mutations of optimised RUBY™ disclosed herein, and furthermore with the improved purity mutations of optimised RUBY™. However, the following are particularly preferred combinations of primary mutations and secondary mutations.

As discussed above, any combination of the primary mutations and secondary mutations can be used in the same polypeptide, such as any one or more both of the primary mutations in (a) and (b), being combined with any one or more of the following secondary mutations in (c) and (d), or variations described herein:
(a) T187E in the CH1 domain (according to EU numbering system); and/or
(b) S114A in the CKappa domain; and
(c) Y180T in the CH1 domain (according to EU numbering system); and/or
(d) T109P and/or A111V CKappa domain (according to EU or Kabat numbering systems).

Accordingly, in a particular embodiment, a polypeptide with combined primary mutations and secondary mutations could include the following mutations:
in the CH1 domain: Y180T and T187E (according to EU numbering system);
in the CKappa domain: T109P and/or A111V, and S114A (according to EU or Kabat numbering systems).

In one embodiment, the primary mutations are a primary mutation pair at position 114 of the CKappa domain and/or at position 187 of the CH1 domain;
and the secondary mutations are a secondary mutation pair selected from the list consisting of:
   position 126 of the VL domain and/or position 180 of the CH1 domain;
   position 126 of the VL domain and/or position 183 of the CH1 domain;
   position 126 of the VL domain and/or position 188 of the CH1 domain;
   position 109 of the CKappa domain and/or position 180 of the CH1 domain;
   position 109 of the CKappa domain and/or position 183 of the CH1 domain;
   position 109 of the CKappa domain and/or position 188 of the CH1 domain;
   position 111 of the CKappa domain and/or position 180 of the CH1 domain;
   position 111 of the CKappa domain and/or position 183 of the CH1 domain; and
   position 111 of the CKappa domain and/or position 188 of the CH1 domain.

In a preferred embodiment, the primary mutations are a primary mutation pair of S114A of the CKappa domain and/or of T187E of the CH1 domain;
and the secondary mutations are a secondary mutation pair selected from the list comprising of:
   mutation I126A in the VL domain and/or mutation Y180I in the CH1 domain;
   mutation I126A in the VL domain and/or mutation Y180W in the CH1 domain;
   mutation I126A in the VL domain and/or mutation S183T in the CH1 domain;
   mutation I126A in the VL domain and/or mutation V188G in the CH1 domain;
   mutation I126G in the VL domain and/or mutation Y180I in the CH1 domain;
   mutation I126G in the VL domain and/or mutation Y180W in the CH1 domain;
   mutation I126G in the VL domain and/or mutation S183T in the CH1 domain;
   mutation I126G in the VL domain and/or mutation V188G in the CH1 domain;
   mutation I126S in the VL domain and/or mutation Y180I in the CH1 domain;
   mutation I126S in the VL domain and/or mutation Y180W in the CH1 domain;
   mutation I126S in the VL domain and/or mutation S183T in the CH1 domain;
   mutation I126S in the VL domain and/or mutation V188G in the CH1 domain;
   mutation T109P in the CKappa domain and/or mutation Y180I in the CH1 domain;
   mutation T109P in the CKappa domain and/or mutation Y180W in the CH1 domain;
   mutation T109P in the CKappa domain and/or mutation S183T in the CH1 domain;
   mutation T109P in the CKappa domain and/or mutation V188G in the CH1 domain;
   mutation A111V in the CKappa domain and/or mutation Y180I in the CH1 domain;
   mutation A111V in the CKappa domain and/or mutation Y180W in the CH1 domain;
   mutation A111V in the CKappa domain and/or mutation S183T in the CH1 domain; and
   mutation A111V in the CKappa domain and/or mutation V188G in the CH1 domain, preferably:
   mutation I126S in the VL domain and/or mutation S183T in the CH1 domain
   mutation I126S in the VL domain and/or mutation V188G in the CH1 domain
   mutation T109P in the CKappa domain and/or mutation S183T in the CH1 domain;
   mutation T109P in the CKappa domain and/or mutation V188G in the CH1 domain;
   mutation A111V in the CKappa domain and/or mutation S183T in the CH1 domain; and
   mutation A111V in the CKappa domain and/or mutation V188G in the CH1 domain, most preferably
   mutation T109P in the CKappa domain and/or mutation S183T in the CH1 domain.

TABLE H
Combinations of primary and secondary mutations in CKappa and VL,
or CKappa/CLambda, amino acid sequences relevant to the invention.
Secondary mutation at position 109 of the CKappa/CLambda domain is at X¹⁰⁹,
secondary mutation at position 111 of the CKappa/CLambda domain is at X¹¹¹, primary
mutation at position 114 of the CKappa/CLambda domain is at X¹¹⁴ (according to the
EU or Kabat numbering systems), and secondary mutation at position 126 of the VL
domain is at X¹²⁶ (according to the IMGT numbering system). As explained for Table
A, the last VL domain amino acid is italicised and the first CKappa/CLambda domain
amino acid is emboldened.

| CKappa + VL, or | | SEQ ID |
|---|---|---|
| CLambda + VL | Amino acid sequence | NO: |
| CKappa + VL | TFGQGTKVX¹²⁶IKRX¹⁰⁹VX¹¹¹APX¹¹⁴VFIFPPSDEQL | 176 |
| CKappa + VL | TFGQGTKLX¹²⁶IKRX¹⁰⁹VX¹¹¹APX¹¹⁴VFIFPPSDEQL | 177 |
| CKappa + VL | TFGPGTKVX¹²⁶IKRX¹⁰⁹VX¹¹¹APX¹¹⁴VFIFPPSDEQL | 178 |
| CKappa + VL | TFGGGTKVX¹²⁶IKRX¹⁰⁹VX¹¹¹APX¹¹⁴VFIFPPSDEQL | 179 |
| CKappa + VL | TFGQGTRLX¹²⁶IKRX¹⁰⁹VX¹¹¹APX¹¹⁴VFIFPPSDEQL | 180 |
| CKappa + VL | TFGAGTKLX¹²⁶IKRX¹⁰⁹VX¹¹¹APX¹¹⁴VFIFPPSDEQL | 288 |
| CKappa + VL | VX¹²⁶IKRX¹⁰⁹VX¹¹¹APX¹¹⁴V | 181 |
| CKappa + VL | LX¹²⁶IKRX¹⁰⁹VX¹¹¹APX¹¹⁴V | 182 |
| CKappa | RX¹⁰⁹VX¹¹¹APX¹¹⁴V | 183 |
| CKappa | VX¹¹¹APX¹¹⁴V | 184 |
| CLambda + VL | VFGTGTKVTVLGQX¹⁰⁹KX¹¹¹NPX¹¹⁴VTLFPPSSEEL | 185 |
| CLambda + VL | VFGTGTKVTVLGQX¹⁰⁹KX¹¹¹APX¹¹⁴VTLFPPSSEEL | 186 |
| CLambda + VL | VFGGGTKLTVLGQX¹⁰⁹KX¹¹¹NPX¹¹⁴VTLFPPSSEEL | 187 |
| CLambda + VL | VFGGGTKLTVLGQX¹⁰⁹KX¹¹¹APX¹¹⁴VTLFPPSSEEL | 188 |
| CLambda + VL | VFGGGTQLIIZGQX¹⁰⁹KX¹¹¹APX¹¹⁴VTLFPPSSEEL | 189 |
| CLambda + VL | VFGGGTQLIILGQX¹⁰⁹KX¹¹¹APX¹¹⁴VTLFPPSSEEL | 190 |
| CLambda + VL | VFGEGTELTVLGQX¹⁰⁹KX¹¹¹NPX¹¹⁴VTLFPPSSEEL | 191 |
| CLambda + VL | VFGEGTELTVLGQX¹⁰⁹KX¹¹¹APX¹¹⁴VTLFPPSSEEL | 192 |
| CLambda + VL | VFGGGTQLTVLGQX¹⁰⁹KX¹¹¹NPX¹¹⁴VTLFPPSSEEL | 193 |
| CLambda + VL | VFGGGTQLTVLGQX¹⁰⁹KX¹¹¹APX¹¹⁴VTLFPPSSEEL | 194 |
| CLambda | GQX¹⁰⁹VX¹¹¹NPX¹¹⁴V | 195 |
| CLambda | GQX¹⁰⁹VX¹¹¹APX¹¹⁴V | 196 |
| CLambda | KX¹¹¹NPX¹¹⁴V | 197 |
| CLambda | KX¹¹¹APX¹¹⁴V | 198 |

X¹¹⁴ is any amino acid but preferably T or S, and it is preferably replaced by A.
X¹⁰⁹ is any amino acid but preferably T, and it is preferably replaced by P.
X¹¹¹ is any amino acid but preferably A, and it is preferably replaced by R or T or W or V.
X¹²⁶ is any amino acid but preferably I, and it is preferably replaced by A or G or H or N or P or Q or S or T.

In one embodiment, the primary mutation at position 114 is at position X¹¹⁴ and the secondary mutation at position 109 in the CKappa or CLambda domain is at position X¹⁰⁹ and/or the secondary mutation at position 111 in the CKappa or CLambda domain is at position X¹¹¹ and/or the secondary mutation at position 126 in the VL domain is at position X¹²⁶ in an amino acid sequence selected from Table H. In a particular embodiment, the primary mutation at position 114 is at position X¹¹⁴ and the secondary mutation at position 109 in the CKappa domain is at position X¹⁰⁹ and the secondary mutation at position 111 in the CKappa domain is at position X¹¹¹ and the secondary mutation at position 126 in the VL domain is at position X¹²⁶ in an amino acid sequence selected from Table H. In a further embodiment, the primary mutation at position 114 is at position X¹¹⁴ and the secondary mutation at position 109 in the CKappa or CLambda domain is at position X¹⁰⁹ and the secondary mutation at position 111 in the CKappa or CLambda domain is at position X¹¹¹ in an amino acid sequence selected from Table H. In a further embodiment, the primary mutation at position 114 is at position X¹¹⁴ and the secondary mutation at position 109 in the CKappa domain is at position X¹⁰⁹ and the secondary mutation at position 126 in the VL domain is at position X¹²⁶ in an amino acid sequence selected from Table H. In a further embodiment, the primary mutation at position 114 is at position X¹¹⁴ and the secondary mutation at position 111 in the CKappa domain is at position X¹¹¹ and the secondary mutation at position 126 in the VL domain is at position X¹²⁶ in an amino acid sequence selected from Table H. In a particular embodiment, the primary mutation at position 114 is at position X¹¹⁴ and the secondary mutation at position 109 in the CKappa or CLambda domain is at position X¹⁰⁹ in an amino acid sequence selected from Table H. In an alternative particular embodiment, the primary mutation at position 114 is at position X¹¹⁴ and the secondary mutation at position 111 in the CKappa or CLambda domain is at position X¹¹¹. In an alternative particular embodiment, the primary mutation at position 114 is at position X¹¹⁴ and the secondary mutation at position 126 in the VL domain is at position X¹²⁶ in an amino acid sequence selected from Table H. As will be appreciated, where a mutation is not present at one or two of positions X¹⁰⁹, X¹¹¹ and X¹²⁶ the amino acid in the same position from the corresponding wildtype amino acid sequence in Table A will be included.

In one embodiment, the amino acid sequence comprises a sequence comprising X¹¹⁴ and X¹⁰⁹ and/or X¹¹¹ and/or X¹²⁶ which has a 10% or more; 20% or more; 30% or more; 40% or more; 50% or more; 60% or more; 70% or more; 80% or more; 90% or more; or 95% or more sequence identity to an amino acid sequence selected from Table H. As will be appreciated, where a mutation is not present at one or two of positions X¹⁰⁹, X¹¹¹ and X¹²⁶ the amino acid in the same position from the corresponding wildtype amino acid sequence in Table A will be included.

TABLE I
Secondary mutations in CH1 amino acid sequences relevant to the
invention. Position 180 is at X¹⁸⁰ and position 183 is at X¹⁸³
and position 188 is at X¹⁸⁸, according to the EU numbering system.

| Fc Isotype | Amino acid sequence | SEQ ID NO: |
|---|---|---|
| IgG1 | AVLQSSGLX¹⁸⁰SLX¹⁸³SVVX¹⁸⁷X¹⁸⁸PSSSLGTQTYICNV | 199 |
| IgG2 | AVLQSSGLX¹⁸⁰SLX¹⁸³SVVX¹⁸⁷X¹⁸⁸PSSNFGTQTYTCNV | 200 |
| IgG3 | AVLQSSGLX¹⁸⁰SLX¹⁸³SVVX¹⁸⁷X¹⁸⁸PSSSLGTQTYTCNV | 201 |
| IgG4 | AVLQSSGLX¹⁸⁰SLX¹⁸³SVVX¹⁸⁷X¹⁸⁸PSSSLGTKTYTCNV | 202 |
| IgG1/IgG2/ | LX¹⁸⁰SLX¹⁸³SVVX¹⁸⁷X¹⁸⁸P | 203 |
| IgG3/IgG4 | | |

X¹⁸⁷ is any amino acid but preferably T, and it is preferably replaced by E or D
X¹⁸⁰ is any amino acid but preferably Y, and it is preferably replaced by A or G or I or N or S or Tor Vor W
X¹⁸³ is any amino acid but preferably S, and it is preferably replaced by N or T
X¹⁸⁸ is any amino acid but preferably V, and it is preferably replaced by G

In one embodiment, in the CH1 domain the primary mutation at position 187 is at position X¹⁸⁷ and the secondary mutations at position 180 is at position X¹⁸⁰ and/or position 183 is at position X¹⁸³ and/or position 188 is at position X¹⁸⁸ in the amino acid sequence selected from Table H. selected from Table H. In one embodiment, in the CH1 domain the primary mutation at position 187 is at position X¹⁸⁷ and the secondary mutations at position 180 is at position X¹⁸⁰ and position 183 is at position X¹⁸³ and position 188 is at position X¹⁸⁸ in the amino acid sequence selected from Table I. In one embodiment, in the CH1 domain the primary mutation at position 187 is at position X¹⁸⁷ and the secondary mutations at position 180 is at position X¹⁸⁰ and position 183 is at position X¹⁸³ in the amino acid sequence selected from Table I. In one embodiment, in the CH1 domain the primary mutation at position 187 is at position X¹⁸⁷ and the secondary mutations at position 180 is at position X¹⁸⁰ and position 188 is at position X¹⁸⁸ in the amino acid sequence selected from Table I. In one embodiment, in the CH1 domain the primary mutation at position 187 is at position X¹⁸⁷ and the secondary mutations at position 183 is at position X¹⁸³ and position 188 is at position X¹⁸⁸ in the amino acid sequence selected from Table I. As will be appreciated, where a mutation is not present at one or two of positions X¹⁸⁰, X¹⁸³ and X¹⁸⁸ the amino acid in the same position from the corresponding wildtype amino acid sequence in Table C will be included.

In one embodiment, the amino acid sequence comprises a sequence comprising X¹⁸⁷ and X¹⁸⁰ and/or X¹⁸³ and/or X¹⁸⁸ which has a 10% or more; 20% or more; 30% or more; 40% or more; 50% or more; 60% or more; 70% or more; 80% or more; 90% or more; or 95% or more sequence identity to the amino acid sequence selected from Table I. As will be appreciated, where a mutation is not present at one or two of positions X¹⁸⁰, X¹⁸³ and X¹⁸⁸ the amino acid in the same position from the corresponding wildtype amino acid sequence in Table C will be included.

As discussed above, it will be appreciated that although the positioning of the mutation in the amino acid sequences described herein is with reference to a numerical value, that is to allow a cross-reference between these particular embodiments and other embodiments further discussing those mutations. Therefore, it is encompassed that the mutations as described within the amino acid sequences disclosed may also be included in the context of that sequence but not at the overall position specified by the associated number. Accordingly, and as an example, by in the CH1 domain the primary mutation at position 187 is at position X¹⁸⁷ and the secondary mutations at position 180 is at position X¹⁸⁰ and/or position 183 is at position X¹⁸³ and/or position 188 is at position X¹⁸⁸ in the amino acid sequence AVLQSSGLX¹⁸⁰SLX¹⁸³SVVX¹⁸⁷X¹⁸⁸PSSSLGTQTYICNV (SEQ ID NO: 199), it is also included that the primary mutation is at position X^{P} and secondary mutation can be one or more of positions X⁵ in the amino acid sequence AVLQSSGLX^{s}SLX^{s}SVVX^{p}X^{s}PSSSLGTQTYICNV (SEQ ID NO: 199).

In one embodiment, the primary mutations are a primary mutation pair at position 114 of the CKappa domain and at position 187 of the CH1 domain;
the secondary mutations are at two or more of the following positions: 180 in the CH1 domain; 183 in the CH1 domain; 188 in the CH1 domain; 111 in the CKappa or CLambda (preferably CKappa) domain; 109 in the CKappa or CLambda (preferably CKappa) domain; and 126 in the VL domain;
and/or the polypeptide further comprises a mutation at position 65 in the VH domain.

In a fourth aspect, the invention provides a bispecific antibody comprising:
(a) an immunoglobulin molecule having specificity for a first antigen, the immunoglobulin molecule comprising two first heavy chain regions and two first light chain regions; and
(b) two Fab fragment having specificity for a second antigen, the Fab fragment comprising a second heavy chain region and a second light chain region;
wherein the second light chain region is fused to the C-terminus of the first heavy chain region;
wherein the polypeptide comprises one or more primary mutations to promote an association of the heavy chain region with the light chain region, and wherein such mutations increase the predicted risk of inducing immunogenic responses and/or increase the immunogenicity of the polypeptide; and
wherein the polypeptide comprises one or more secondary mutations to reduce or avoid the predicted risk of inducing immunogenic responses and/or immunogenicity caused by the one or more primary mutations.

In a preferred embodiment of the fourth aspect invention, the polypeptide can comprise any of the mutations described in the first and/or second aspect of the invention, as well as any other features described therein.

A particular embodiment of the fourth aspect of the invention provides a bispecific antibody comprising:
(a) an immunoglobulin molecule having specificity for a first antigen, the immunoglobulin molecule comprising two first heavy chain regions and two first light chain regions; and
(b) two Fab fragment having specificity for a second antigen, the Fab fragment comprising a second heavy chain region and a second light chain region;
wherein the second light chain region is fused via a linker to the C-terminus of the first heavy chain region;
wherein the bispecific antibody comprises: two H1 chains, each comprising a first heavy chain region of the immunoglobulin, a linker and a second light chain region of a Fab; two L1 chains, each comprising a first light chain region of the immunoglobulin; and two H2 chains comprising a second heavy chain region of a Fab;
wherein the bispecific antibody comprises a mutation at position 114 in the CKappa or CLambda (preferably CKappa) domain (according to the EU or Kabat numbering systems) of the two H1 chains and/or at position 187 in the CH1 domain (according to EU numbering system) of the two H2 chains; and
wherein the bispecific antibody comprises two or more mutations of the following positions: 180 in the CH1 domain of the two H2 chains; 183 in the CH1 domain of the two H2 chains; 188 in the CH1 domain of the two H2 chains; 111 in the CKappa or CLambda (preferably CKappa) domain of the two H1 chains; 109 in the CKappa or CLambda (preferably CKappa) domain of the two H1 chains; and 126 in the VL domain of the two H1 chains.

In a fifth aspect, the invention provides a bispecific antibody comprising:
(a) an immunoglobulin molecule having specificity for a first antigen, the immunoglobulin molecule comprising two first heavy chain regions and two first light chain regions; and
(b) two Fab fragment having specificity for a second antigen, the Fab fragment comprising a second heavy chain region and a second light chain region;
wherein the second light chain region is fused to the C-terminus of the first heavy chain region;
wherein the polypeptide comprises one or more primary mutations to promote an association of the heavy chain region with the light chain region, and wherein such mutations increase the predicted risk of inducing immunogenic responses and/or increase the immunogenicity of the polypeptide;
wherein the polypeptide comprises one or more secondary mutations to reduce or avoid the predicted risk of inducing immunogenic responses and/or immunogenicity caused by the one or more primary mutations;
and wherein the second heavy chain region comprises a VH domain, wherein the VH domain comprises a protein A binding site, and wherein the VH domain comprises a mutation that reduces or stops the binding of protein A to the protein A binding site.

In a fifth embodiment of the third aspect invention, the polypeptide can comprise any of the mutations described in the first and/or second aspect of the invention, as well as any other features described therein.

A particular embodiment of the fifth aspect of the invention provides a bispecific antibody comprising:
(a) an immunoglobulin molecule having specificity for a first antigen, the immunoglobulin molecule comprising two first heavy chain regions and two first light chain regions; and
(b) two Fab fragment having specificity for a second antigen, the Fab fragment comprising a second heavy chain region and a second light chain region;
wherein the second light chain region is fused via a linker to the C-terminus of the first heavy chain region;
wherein the bispecific antibody comprises: two H1 chains, each comprising a first heavy chain region of the immunoglobulin, a linker and a second light chain region of a Fab; two L1 chains, each comprising a first light chain region of the immunoglobulin; and two H2 chains comprising a second heavy chain region of a Fab;
wherein the bispecific antibody comprises a mutation at position 114 in the CKappa or CLambda (preferably CKappa) domain (according to the EU or Kabat numbering systems) of the two H1 chains and/or at position 187 in the CH1 domain (according to EU numbering system) of the two H2 chains;
wherein the bispecific antibody comprises two or more mutations of the following positions: 180 in the CH1 domain of the two H2 chains; 183 in the CH1 domain of the two H2 chains; 188 in the CH1 domain of the two H2 chains; 111 in the CKappa or CLambda (preferably CKappa) domain of the two H1 chains; 109 in the CKappa or CLambda (preferably CKappa) domain of the two H1 chains; and 126 in the VL domain of the two L1 chains;
and wherein the two second heavy chain regions (i.e. H2) comprises a VH domain comprising a mutation at position 65 (according to the IMGT numbering system).
   Additional Mutations that can be Combined with the Mutations Described Herein

In addition to the “primary mutations”, “secondary mutations” and “improved purity mutations” described herein, the following “additional mutations” can also be included in the polypeptides of the invention. These additional mutations are the mutations described for the “RUBY™ format” in pending UK patent application 1820556.7 and the PCT application WO 2020/127354, which are not the primary mutations described here.

In one embodiment, the additional mutations are one or more mutations that promote association of the heavy chain polypeptide of the immunoglobulin with the light chain polypeptide of the immunoglobulin and/or to promote association of the heavy chain polypeptide of the Fab with the light chain polypeptide of the Fab.

In one embodiment the one or more additional mutations prevent the formation of aggregates and a Fab by-product.

It will be appreciated by persons skilled in the art, that in one embodiment the additional mutations may prevent the formation of aggregates and/or a Fab by-product by generating steric hindrance and/or incompatibility between charges.

By “steric hindrance” we mean the slowing of a reaction due to steric bulk, i.e. the size of an amino acid molecule prevents association of two protein surfaces that may otherwise occur if a smaller amino acid is present.

By “incompatibility between charges” we mean that an unwanted product will not form as the charges are incompatible and prevent the product from forming, e.g. there may be two negatively charged portions which repel and prevent an unwanted product from forming.

As described above, said additional mutations limit the formation of a Fab by-product and/or aggregates by, for example, creating surfaces that limit the formation of aggregates or by-product Fab fragments. In one embodiment, the primary mutations prevent formation of a Fab by-product by generating steric hindrance and/or incompatibility between charges (leading to charge incompatibility of wrong chains).

The primary mutations may also promote interactions between correct chains (i.e. between the first heavy chain polypeptide and the first light chain polypeptide, and/or between the second heavy chain polypeptide and the second light chain polypeptide) by, for example, creating salt or disulphide bridges.

Thus, the additional mutations may favour formation of a bispecific polypeptide.

In one embodiment, the percentage of aggregates formed during manufacturing is less than or equal to 25%. Optionally the percentage of aggregates is less than or equal to 20%, 17.5%, 15%, 13.5% or 10%. Preferably the percentage of aggregates is less than 10%. Optionally these measurements are carried out when the chains of the bispecific polypeptide are transfected at equal ratios, e.g. at a ratio of 1:1:1 when 3 chains are used during production.

Alternatively, the chain transfection ratio may be optimised. Optionally the % of aggregates when the chain transfection ratio is optimised may be less than or equal to 3.5%, 3%, 2.5% or 2%.

In one embodiment, the bispecific polypeptide comprises one or more mutation pairs each comprising two functionally compatible mutations.

By “functionally compatible mutations” we mean the mutations have complementary functions, e.g. one mutation of the pair (in one chain) may be a mutation that forms a positively charged region, and the other mutation (in another chain) forms a negatively charged region. Together these mutations act in a functionally compatible way promoting association of the respective chains.

In one embodiment, the bispecific polypeptide comprises one or more additional mutation pairs in one or more of the following region groups:
(a) the CH1 and CKappa or CLambda region of the immunoglobulin; and/or
(b) the CH1 and CKappa or CLambda region of the Fab; and/or
(c) the VL and VH regions of the immunoglobulin; and/or
(d) the VL and VH regions of the Fab.

Thus, in one embodiment, the additional mutation pairs are in the CH1 and CKappa or CLambda regions of the Fab and/or the immunoglobulin, and the primary mutation pairs are selected from:
(a) cavity and protruding surface mutations (i.e. steric mutations); and/or
(b) hydrophobic swap mutations; and/or
(c) charged mutations (i.e. salt mutations); and/or
(d) mutations resulting in the formation of a disulphide bridge.

The additional mutation pairs may alternatively or additionally be in the VH and VL regions of the Fab and/or the immunoglobulin, the primary mutation pairs in the VH and VL regions are selected from:
(a) charged mutations (i.e. salt mutations); and/or
(b) double charged mutations; and/or
(c) mutations resulting in the formation of a disulphide bridge.

In one embodiment of the invention the additional mutations are at positions selected from the group consisting of:
(a) one or more of the following positions in the CH1 domain: 168, 170, and 145 (according to EU numbering system); and/or
(b) a position selected from the one or more of the following position ranges in the CKappa or CLambda domain: position 132 to 138, position 173 to 179, position 130 to 136, position 111 to 117 and position 134 to 140 (according to EU numbering system); and/or
(c) a position selected from one or more of the following position ranges in the VL: position 41 to 47, position 117 to 123 and position 46 to 52 (according to IMGT numbering system); and/or
(d) a position selected from one or more of the following position ranges in the VH: position 41 to 47, position 46 to 52 and position 117 to 123 (according to IMGT numbering system).

In one embodiment of the invention the additional mutations are at positions selected from the group consisting of:
(a) one or more of the following positions in the CH1 domain: 168, 170, and 145 (according to EU numbering system); and/or
(b) a position selected from the one or more of the following position ranges in the CKappa or CLambda domain: position 132 to 138, position 173 to 179, position 130 to 136, position 111 to 117 and position 134 to 140 (according to Kabat numbering system); and/or
(c) a position selected from one or more of the following position ranges in the VL: position 41 to 47, position 117 to 123 and position 46 to 52 (according to IMGT numbering system); and/or
(d) a position selected from one or more of the following position ranges in the VH: position 41 to 47, position 46 to 52 and position 117 to 123 (according to IMGT numbering system).

In one embodiment of the invention the additional mutations are at positions selected from the group consisting of:
(a) one or more of the following positions in the CH1 domain: 168, 170, and 145 (according to EU numbering system); and/or
(b) a position selected from the one or more of the following position ranges in the CKappa or CLambda domain: position 132 to 138, position 173 to 179, position 130 to 136, position 111 to 117 and position 134 to 140 (according to EU numbering system); and/or
(c) a position selected from one or more of the following position ranges in the VL: position 41 to 47, position 117 to 123 and position 46 to 52 (according to IMGT numbering system); and/or
(d) a position selected from one or more of the following position ranges in the VH: position 41 to 47, position 46 to 52 and position 117 to 123 (according to IMGT numbering system).

One mutation in each of the ranges given above will be the relevant functional mutation as it will be a position that makes contact with the amino acid in the corresponding domain/chain, and is therefore the relevant interface between chains.

It will therefore be appreciated by persons skilled in the art that additional mutations in the position ranges given above are suitable, as the relevant functional feature is whether the position contacts a corresponding position on the other chain, i.e. a position in the VH chain that contacts a corresponding position in a VL chain is the relevant position, or a position in a CLambda that contacts a position in a CH1 chain is the relevant position.

In a preferred embodiment of the invention the additional mutations are at positions selected from the group consisting of:
(a) one or more of the following positions in the CH1 domain: 168, 170, and 145 (according to EU numbering system); and/or
(b) one or more of the following positions in the CKappa or CLambda domain: 114, 133, 135, 137, and 176 (according to Kabat numbering system); and/or
(c) one or more of the following positions in the VL domain: 44, 49, and 120 (according to IMGT numbering system); and/or
(d) one or more of the following positions in the VH domain: 44, 49, and 120 (according to IMGT numbering system).

In one embodiment of the invention the additional mutations are at positions selected from the group consisting of:
(a) one or more of the following positions in the CH1 domain: 168, 170, and 145 (according to EU numbering system); and/or
(b) one or more of the following positions in the CKappa or CLambda domain: 114, 133, 135, 137, and 176 (according to EU numbering system); and/or
(c) one or more of the following positions in the VL domain: 44, 49, and 120 (according to IMGT numbering system); and/or
(d) one or more of the following positions in the VH domain: 44, 49, and 120 (according to IMGT numbering system).

In one embodiment the additional mutations are selected from the group consisting of:
VH X44R/E/D/K, X49C, X120K
VL X44R/E/D/K, X49D X120C
CH1 X168A/G, X170G/A, and X145Q
CKappa/CLambda X114A, X133T, X135Y/W, X137K/R/H, X176W/V/Y
*numbering according to IMGT system for VH/VL domains and according to EU numbering system for constant domains
*X refers to any amino acid

The use of “/” in the context of discussing mutations is to illustrate alternative possible amino acids; for example, “X44R/E/D/K” indicates that R or E or D or K can be included at position 44, as a substitute for the amino acid “X”.

In one embodiment the additional mutations are selected from the group consisting of:
VH X44R/E/D/K, X49C, X120K
VL X44R/E/D/K, X49D X120C
CH1 X168A/G, X170G/A, X145Q
CKappa/CLambda X114A, X133T, X135Y/W, X137K/R/H, X176W/V/Y
*numbering according to IMGT system for VH/VL domains and according to Kabat numbering system for constant domains
*X refers to any amino acid

In one embodiment the additional mutations are selected from the group consisting of:
VH Q44X, G49X, Q120X
VL Q44X, A49X, Q120X
CH1 H168X, F170X, L145X,
CKappa/CLambda S/T114X, V133X, L135X, N/S137X, S176X
*numbering according to IMGT system for VH/VL domains and according to EU numbering system for constant domains
*X refers to any amino acid

In one embodiment the additional mutations are selected from the group consisting of:
VH Q44X, G49X, Q120X
VL Q44X, A49X, Q120X
CH1 H168X, F170X, L145X
CKappa/CLambda S/T114X, V133X, L135X, N/S137X, S176X
*numbering according to IMGT system for VH/VL domains and according to Kabat numbering system for constant domains
*X refers to any amino acid

In one embodiment of the invention, the additional mutations are at positions selected from the group consisting of:
(a) one or more of the following positions in the CH1 domain: H168, F170, and L145 (according to EU numbering system); and/or
(b) one or more of the following positions in the CKappa domain: L135, S176, V133, and N137 (according to EU numbering system) and/or one or more of the following positions in the CLambda domain: L135, S176, V133, and S137 (according to Kabat numbering system); and/or
(c) one or more of the following positions in the VL domain: Q44, Q120 and A49 (according to IMGT numbering system); and/or
(d) one or more of the following positions in the VH domain: Q44, G49 and Q120 (according to IMGT numbering system).

For example, the additional mutations may be selected from the group consisting of:
(a) one or more of the following mutations in the CH1 domain: H168A, F170G, and L145Q (according to EU numbering system); and/or
(b) one or more of the following mutations in the CKappa domain: L135Y, S176W, V133T, S176V, and N137K (according to EU numbering system) and/or one or more of the following mutations in the CLambda domain: L135Y, S176W, V133T, S176V, and S137K (according to Kabat numbering system); and/or
(c) one or more of the following mutations in the VL domain: Q44R, Q44E, Q120C, Q44D and A49D (according to IMGT numbering system); and/or
(d) one or more of the following mutations in the VH domain: Q44E, Q44R, G49C, Q44K and Q120K (according to IMGT numbering system).

In a preferred embodiment, in the bispecific antibody described herein the additional mutation at position 44 in the VH domain is in the “H1” chain as described herein and/or the “H2” chain as described here.

In a preferred embodiment, in the bispecific antibody described herein the additional mutation at position 44 in the VL domain is in the “H1” chain as described herein and/or the “L1” chain as described herein.

In a preferred embodiment, in the bispecific antibody described herein the additional mutation at position 49 in the VH domain is in the “H1” chain as described herein and/or the “H2” chain as described here.

In a preferred embodiment, in the bispecific antibody described herein the additional mutation at position 49 in the VL domain is in the “H1” chain as described herein and/or the “L1” chain as described herein.

In a preferred embodiment, in the bispecific antibody described herein the additional mutation at position 120 in the VH domain is in the “H1” chain as described herein and/or the “H2” chain as described here.

In a preferred embodiment, in the bispecific antibody described herein the additional mutation at position 120 in the VL domain is in the “H1” chain as described herein and/or the “L1” chain as described herein.

In a preferred embodiment, in the bispecific antibody described herein the additional mutation at position 168 in the CH1 domain is in the “H1” chain as described herein and/or the “H2” chain as described here.

In a preferred embodiment, in the bispecific antibody described herein the additional mutation at position 170 in the CH1 domain is in the “H1” chain as described herein and/or the “H2” chain as described here.

In a preferred embodiment, in the bispecific antibody described herein the additional mutation at position 145 in the CH1 domain is in the “H1” chain as described herein and/or the “H2” chain as described here.

In a preferred embodiment, in the bispecific antibody described herein the additional mutation at position 133 in the CKappa or CLambda (preferably CKappa) domain is in the “H1” chain as described herein and/or the “L1” chain as described herein.

In a preferred embodiment, in the bispecific antibody described herein the additional mutation at position 135 in the CKappa or CLambda (preferably CKappa) domain is in the “L1” chain as described herein and/or the “H1” chain as described here.

In a preferred embodiment, in the bispecific antibody described herein the additional mutation at position 176 in the CKappa or CLambda (preferably CKappa) domain is in the “L1” chain as described herein and/or the “H1” chain as described here.

In a preferred embodiment, in the bispecific antibody described herein the additional mutation at position 137 in the CKappa or CLambda (preferably CKappa) domain is in the “H1” chain as described herein and/or the “L1” chain as described here.

In particular embodiment, the additional mutations are the following:
at position 44 (such as Q44R and/or Q44E) of the VH domain;
at position 44 (such as Q44R and/or Q44E) of the VL domain;
at position 168 and 170 (such as H168A and F170G) of the CH1 domain; and
at positions 137, 135 and 176 (such as N137K or S137K, L135Y and S176W) of the CKappa or CLambda (preferably CKappa) domain.

In a particularly preferred embodiment, in the bispecific antibody described herein the additional mutations are the following:
at position 44 (such as Q44R) of the VH domain of the “H1” chain;
at position 44 (such as Q44R) of the VL domain of the “H1” chain;
at position 44 (such as Q44E) of the VH domain of the “H2” chain;
at position 44 (such as Q44E) of the VL domain of the “L1” chain;
at position 168 and 170 (such as H168A and F170G) of the CH1 domain of the “H1” chain;
at position 137 (such as N137K or S137K) of the CKappa or CLambda (preferably CKappa) domain of the “H1” chain; and
at position 135 and 176 (such as L135Y and S176W) of the CKappa or CLambda (preferably CKappa) domain of the “L1” chain.

### Polypeptide Binding Targets

In one embodiment, the bispecific polypeptide is tetravalent, capable of binding bivalently to each of the two antigens.

In one embodiment, the bispecific polypeptide comprises an immunoglobulin arranged as an antibody with two arms and therefore two binding sites for the first antigen, and two of the Fab fragments, each providing a binding site for the second antigen. Thus, there are two binding sites for the first antigen and two binding sites for the second antigen.

In one embodiment, the antigen (such as the first and/or second antigen) is an immunomodulator.

By “immunomodulator” we mean a target which is capable of modifying the immune response or the functioning of the immune system. In one embodiment, the immunomodulator is a checkpoint molecule. By “checkpoint molecule” we mean a regulator of the immune system.

It will be appreciated by persons skilled in the art that the checkpoint molecule (such as CD40) may either be a stimulatory or inhibitory checkpoint molecule.

In one embodiment, the antigen (such as the first and/or second antigen) is a tumour cell-associated antigen (TAA).

Accordingly, the tumour cell-associated antigen may be selected from the group consisting of:
a) products of mutated oncogenes and tumour suppressor genes;
b) overexpressed or aberrantly expressed cellular proteins;
c) tumour antigens produced by oncogenic viruses;
d) oncofetal antigens;
e) altered cell surface glycolipids and glycoproteins;
f) cell type-specific differentiation antigens;
g) hypoxia-induced antigens;
h) tumour peptides presented by MHC class I;
i) epithelial tumour antigens;
j) haematological tumour-associated antigens;
k) cancer testis antigens; and
I) melanoma antigens.

In one embodiment, the TAA is EpCAM, 5T4 and/or CEACAM5.

### Variants

The polypeptide or constituent binding domains thereof (such as the EpCAM- or CD40-binding domains) described herein may comprise a variant or a fragment of any of the specific amino acid sequences recited herein, provided that the polypeptide or binding domain retains binding to its target. In one embodiment, the variant of an antibody or antigen-binding fragment may retain the CDR sequences of the sequences recited herein.

A fragment of any one of the heavy or light chain amino acid sequences recited herein may comprise at least 7, at least 8, at least 9, at least 10, at least 12, at least 15, at least 18, at least 20, at least 25, at least 50, at least 60, at least 70, at least 80, at least 90 or at least 100 consecutive amino acids from the said amino acid sequence.

A variant of any one of the heavy or light chain amino acid sequences recited herein may be a substitution, deletion or addition variant of said sequence. A variant may comprise 1, 2, 3, 4, 5, up to 10, up to 20, up to 30 or more amino acid substitutions and/or deletions from the said sequence. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala, A | aliphatic, hydrophobic, neutral | Met, M | hydrophobic, neutral |
| Cys, C | polar, hydrophobic, neutral | Asn, N | polar, hydrophilic, neutral |
| Asp, D | polar, hydrophilic, charged (−) | Pro, P | hydrophobic, neutral |
| Glu, E | polar, hydrophilic, charged (−) | Gln, Q | polar, hydrophilic, neutral |
| Phe, F | aromatic, hydrophobic, neutralArg, | R | polar, hydrophilic, charged (+) |
| Gly, G | aliphatic, neutral | Ser, S | polar, hydrophilic, neutral |
| His, H | aromatic, polar, hydrophilic, | Thr, T | polar, hydrophilic, neutral |
| | charged (+) | | |
| Ile, I | aliphatic, hydrophobic, neutral | Val, V | aliphatic, hydrophobic, neutral |
| Lys, K | polar, hydrophilic, charged(+) | Trp, W | aromatic, hydrophobic, neutral |
| Leu, L | aliphatic, hydrophobic, neutral | Tyr, Y | aromatic, polar, hydrophobic |

Amino acids herein may be referred to by full name, three letter code or single letter code.

Preferred “derivatives” or “variants” include those in which instead of the naturally occurring amino acid the amino acid which appears in the sequence is a structural analogue thereof. Amino acids used in the sequences may also be derivatised or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably, variants have an amino acid sequence which has more than 60%, or more than 70%, e.g. 75 or 80%, preferably more than 85%, e.g. more than 90 or 95% amino acid identity to a sequence as shown in the sequences disclosed herein. This level of amino acid identity may be seen across the full length of the relevant SEQ ID NO sequence or over a part of the sequence, such as across 20, 30, 50, 75, 100, 150, 200 or more amino acids, depending on the size of the full-length polypeptide.

In connection with amino acid sequences, “sequence identity” refers to sequences which have the stated value when assessed using ClustalW (Thompson et al., 1994, Nucleic Acids Res. 22(22):4673-80; the disclosures of which are incorporated herein by reference) with the following parameters: Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatised.

### Functional Properties

It will also be appreciated by persons skilled in the art that the polypeptides of the invention may be defined in relation to its functional properties and effects.

In one embodiment, the polypeptide of the invention is capable of inducing antibody-dependent cell cytotoxicity (ADCC), antibody-dependent cellular phagocytosis (ADCP), complement-dependent cytotoxicity (CDC), and/or apoptosis.

In one embodiment, the polypeptide is capable of inducing:
(a) activation of B cells; and/or
(b) activation of dendritic cells; and/or
(c) activation of cytotoxic T cells, i.e. CD8+ T cells; and/or
(d) activation of helper T cells, i.e. CD4+ T cells; and/or
(e) improved tumour antigen cross-presentation by dendritic cells; and/or
(f) expansion of tumour antigen-specific T cells; and/or
(g) direct tumour cell killing via ADCC and/or via inhibition of tumour growth and survival signals; and/or
(h) anti-angiogenic effects via interaction with endothelial and/or stromal cells; and/or
(i) activation of natural killer cells; and/or
(j) Treg depletion; and/or
(k) reprograming of Tregs into effector T cells; and/or
(l) depletion of tumour myeloid cell populations; and/or
(m) reprogramming of tumour myeloid cell populations; and/or
(n) internalisation of tumour debris by antigen-presenting cells; and/or
(o) internalisation of tumour extracellular vesicles, e.g. exosomes, by antigen-presenting cells; and/or
(p) localization to tumour tissue by binding to tumour cells.

Methodologies for determining whether the polypeptide is capable of inducing the above listed functional effects are known to those skilled in the art and are also exemplified in the Examples of the present application.

The polypeptide may modulate the activity of a cell expressing a T cell target, wherein said modulation is an increase or decrease in the activity of said cell. The cell is typically a T cell. The antibody may increase the activity of a CD4+ or CD8+ effector cell, or may decrease the activity of a regulatory T cell (Treg). In either case, the net effect of the antibody will be an increase in the activity of effector T cells.

Methods for determining a change in the activity of effector T cells are well known and include, for example, measuring for an increase in the level of T cell IL-2 or IFN-γ production or an increase in T cell proliferation in the presence of the antibody relative to the level of T cell IL-2 or IFN-γ production and/or T cell proliferation in the presence of a control. Assays for cell proliferation and/or IL-2 or IFN-γ production are well known and assays are also exemplified in the Examples. Additional methods of determining a change in the activity of effector T cells include measuring the change in expression of e.g. CD25, CD69, ICOS, EOMES, CD107a and/or granzyme B by the T cells, or by measuring the change in proliferation of the T cells by use of e.g. CFSE or by measuring levels of Ki67, both of which can be achieved using flow cytometry-based methods.

Methods for determining a change in the activity of Treg cells are well known and include, for example, measuring the reprogramming of Treg cells to Th cells in in vitro assays by evaluating their production of TGF-β and IFN-γ, or by measuring the Treg cell suppression capacity in in vitro assays where CD3/CD28-stimulated CD4+ T cells are co-cultured with the Treg cells and the proliferation of the CD4+ T cells is evaluated by use of CFSE or other similar proliferation dyes, or by measuring the level of Treg cell differentiation in in vitro assays where CD4+ T cells are cultured in conditions promoting polarization to inducible Treg (iTreg) cells and where the frequency of CD127low FoxP3+ iTreg cells is measured. Additional methods for determining a change in the activity of Treg cells induced by the antibody include ADCC reporter assays to determine the depletion of Treg cells or other ADCC reporter assays where Treg cells are co-cultured with NK cells or macrophages and LDH release or various viability stains are used to determine the depletion of Treg cells.

Methods for determining a change in the activity of NK cells are well known and include, for example, measuring the change in expression of e.g. CD25, CD107a, granzyme B or NKG2D by flow cytometry-based methods or in in vitro assays where the effect of the antibody on the functionality of the NK cells can be measured by the release of cytokines and lytic enzymes, e.g. IFN-γ, TNF-α or perforin.

In one embodiment, the polypeptide is capable of inducing an increase in the activity of an effector T cell, optionally wherein said increase is at least 1.5-fold, 4.5-fold or 7-fold higher than the increase in activity of an effector T cell induced by a combination of the immunoglobulin molecule and Fab fragment administered to the T cell as separate molecules. This can be tested in vitro in T cell activation assays, e.g. by measuring. IL-2 or IFN-γ production. Activation of effector T cells would imply that a tumour-specific T cell response can be achieved in vivo. Further, an anti-tumour response in an in vivo model, such as a mouse model would imply that a successful immune response towards the tumour has been achieved. Thus, this would indicate that the bispecific antibody is capable of inducing tumour immunity.

In one embodiment, the polypeptide induces an increase in the activation of an antigen-presenting cell, such as a B cell or dendritic cell.

It will be appreciated by persons skilled in the art, that said increase in activation may be an increase in the expression of the co-stimulatory molecules CD80 or CD86, or increased IL-12 production, or increased ability to present antigens, e.g. tumor antigens, on MHC class I or II (also by so called cross presentation, whereby an antigen taken up by internalization induced by the bispecific antibody (for example, as induced by the exemplary bispecific CD40-EPCAM antibody) ends up being presented on an MHC class I molecule) to T-cells, generating an enhanced activation of T-cells recognizing said antigen, by the antigen-presenting cell.

In one embodiment, the polypeptide induces an increase in the uptake of tumour debris or tumour extracellular vesicles by an antigen-presenting cell, such as a B cell or dendritic cell.

It will be appreciated by persons skilled in the art, that said increase in uptake may be measured by the co-localization or internalization of the tumour debris or tumour extracellular vesicles by the antigen-presenting cell. The increased uptake of tumour debris or tumour extracellular vesicles by the antigen-presenting cells would subsequently result in a broader T cell repertoire and, thus, more effective T cell-mediated tumour eradication. Methods for determining the expansion of tumour-antigen specific T cells are well known and include, for example, the use of MHC-peptide multimers, e.g. tetramers or pentamers. Such expansion may be measured by inoculating mice with tumours expressing a specific tumour antigen or tumours transfected with a tumour model antigen (e.g. ovalbumin), alternatively by inoculating mice with the same cells that have been heat shocked to induce necrosis, followed by measuring the expansion of tumour antigen-specific T cells by use of various MHC-tumour (model) antigen peptide tetramers or pentamers by flow cytometry-based methods.

In one embodiment, the polypeptide binds to the antigen (such as the first and/or the second antigen) with a K_{D} of less than 100×10⁻⁹M or less than 50×10⁻⁹M or less than 25×10⁻⁹M, preferably less than 10, 9, 8, 7, or 6×10⁻⁹M, more preferably less than 5, 4, 3, 2, or 1×10⁻⁹M, most preferably less than 9×10⁻¹⁰M.

Standard assays to evaluate the binding ability of ligands towards targets are well known in the art, including for example, ELISAs, Western blots, RIAs, and flow cytometry analysis. The binding kinetics (e.g., binding affinity) of the bispecific antibody can also be assessed by standard assays known in the art, such as by Surface Plasmon Resonance analysis (SPR). Such assays are also demonstrated within the Examples of the present application.

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of this dissociation constant can be determined directly by well-known methods and can be computed even for complex mixtures by methods such as those, for example, set forth in Caceci et al. (Byte 9:340-362, 1984; the disclosures of which are incorporated herein by reference). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ system analysis.

A competitive binding assay can be conducted in which the binding of the polypeptide to the target is compared to the binding of the target by another known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 of polypeptides are set out in the Examples.

A polypeptide of the invention is preferably capable of binding to one of its targets with an affinity that is at least two-fold, 10-fold, 50-fold, 100-fold or greater than its affinity for binding to another non-target molecule.

### Polynucleotides, Vectors and Cells

The invention also relates to polynucleotides that encode all or part of a polypeptide of the invention. Thus, a polynucleotide of the invention may encode any polypeptide chain as described herein. The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

A polypeptide of the invention may thus be produced from or delivered in the form of a polynucleotide which encodes and is capable of expressing it.

Polynucleotides of the invention can be synthesised according to methods well known in the art, as described by way of example in Green & Sambrook (2012, Molecular Cloning—a laboratory manual, 4th edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art (see Green & Sambrook, supra).

The invention also includes cells that have been modified to express a bispecific antibody or component polypeptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a polypeptide of the invention include mammalian HEK293T, CHO, HeLa, NS0 and COS cells. Preferably the cell line selected will be one which is not only stable, but also allows for mature glycosylation and cell surface expression of a polypeptide.

Such cell lines of the invention may be cultured using routine methods to produce a polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

In a further aspect, the invention provides a method of manufacturing any of the polypeptides and/or bispecific antibodies disclosed herein.

### Pharmaceutical Formulations, Therapeutic Uses and Patient Groups

In another aspect, the present invention provides compositions comprising molecules of the invention, such as the polypeptides, polynucleotides, vectors and cells described herein. For example, the invention provides a composition comprising one or more molecules of the invention, such as one or more polypeptides of the invention, and at least one pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion). Depending on the route of administration, the polypeptide may be coated in a material to protect the polypeptide from the action of acids and other natural conditions that may inactivate or denature the polypeptide.

Preferred pharmaceutically acceptable carriers comprise aqueous carriers or diluents. Examples of suitable aqueous carriers that may be employed in the compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

A composition of the invention also may include a pharmaceutically acceptable anti-oxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent (e.g. polypeptide) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Particularly preferred compositions are formulated for systemic administration or for local administration. Local administration may be at the site of a tumour or into a tumour-draining lymph node. The composition may preferably be formulated for sustained release over a period of time. Thus, the composition may be provided in or as part of a matrix facilitating sustained release. Preferred sustained release matrices may comprise a montanide or γ-polyglutamic acid (PGA) nanoparticles. Localised release of a polypeptide of the invention, optionally over a sustained period of time, may reduce potential autoimmune side-effects associated with administration of a CTLA-4 antagonist.

Compositions of the invention may comprise additional active ingredients as well as a polypeptide of the invention. As mentioned above, compositions of the invention may comprise one or more polypeptides of the invention. They may also comprise additional therapeutic or prophylactic agents.

Also within the scope of the present invention are kits comprising polypeptides or other compositions of the invention and instructions for use. The kit may further contain one or more additional reagents, such as an additional therapeutic or prophylactic agent as discussed above.

The polypeptides in accordance with the present invention may be used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

In particular, polypeptides of the invention may be useful in the treatment or prevention of cancer. Accordingly, the invention provides a polypeptide of the invention for use in the treatment or prevention of cancer. The invention also provides a method of treating or preventing cancer comprising administering to an individual a polypeptide of the invention. The invention also provides a polypeptide of the invention for use in the manufacture of a medicament for the treatment or prevention of cancer.

The cancer may be prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

A polypeptide of the present invention, or a composition comprising said polypeptide, may be administered via one or more routes of administration using one or more of a variety of methods known in the art. As will be appreciated by the skilled artisan, the route and/or mode of administration will vary depending upon the desired results. Systemic administration or local administration are preferred. Local administration may be at the site of a tumour or into a tumour-draining lymph node. Preferred modes of administration for polypeptides or compositions of the invention include intravenous, intramuscular, intradermal, intraperitoneal, subcutaneous, spinal or other parenteral modes of administration, for example by injection or infusion. The phrase “parenteral administration” as used herein means modes of administration other than enteral and topical administration, usually by injection. Alternatively, a polypeptide or composition of the invention can be administered via a non-parenteral mode, such as a topical, epidermal or mucosal mode of administration.

A suitable dosage of a polypeptide of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular polypeptide employed, the route of administration, the time of administration, the rate of excretion of the polypeptide, the duration of the treatment, other drugs, compounds and/or materials used in combination with the particular compositions employed, the age, sex, weight, condition, general health and prior medical history of the patient being treated, and like factors well known in the medical arts.

A suitable dose of a polypeptide or composition of the invention may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day.

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Polypeptides or compositions may be administered in a single dose or in multiple doses. The multiple doses may be administered via the same or different routes and to the same or different locations. Alternatively, polypeptides or compositions can be administered as a sustained release formulation as described above, in which case less frequent administration is required. Dosage and frequency may vary depending on the half-life of the polypeptide in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Combined administration of two or more agents may be achieved in a number of different ways. In one embodiment, the polypeptide and the other agent may be administered together in a single composition. In another embodiment, the polypeptide and the other agent may be administered in separate compositions as part of a combined therapy. For example, the modulator may be administered before, after or concurrently with the other agent.

A polypeptide or composition of the invention may also be used in a method of increasing the activation of a population of cells expressing the first and second antigen, the method comprising administering to said population of cells a polypeptide or composition of the invention under conditions suitable to permit interaction between said cell and a polypeptide of the invention. The population of cells typically comprises at least some cells which express the first antigen, and at least some cells which express the second antigen. The method is typically carried out ex vivo.

In addition as used in this specification and the appended claims, the singular forms “a”, “an”, and “the” include plural referents unless the content clearly dictates otherwise. Thus, for example, reference to “an antibody” includes “antibodies”, reference to “an antigen” includes two or more such antigens, reference to “a subject” includes two or more such subjects, and the like.

All publications, patents and patent applications cited herein, whether supra or infra, are hereby incorporated by reference in their entirety.

The listing or discussion of an apparently prior-published document in this specification should not necessarily be taken as an acknowledgement that the document is part of the state of the art or is common general knowledge.

The present invention is further illustrated by the following examples which should not be construed as further limiting. The contents of all figures and all references, patents and published patent applications cited throughout this application are expressly incorporated herein by reference.

### BRIEF DESCRIPTION OF THE FIGURES

Preferred, non-limiting examples which embody certain aspects of the invention will now be described, with reference to the following figures:

FIG. 1—Dual ELISA of the single mutant bsAb variants. Mutants evaluated to reduce the in silico predicted MHC class II binding of peptides carrying the S114A mutation are shown in panels A-D and mutants evaluated to reduce the in silico predicted MHC class II binding of peptides carrying the T187E mutation in E-H. As reference the wild-type bsAb AC_05293 was included.

FIG. 2—Dual ELISA of the double mutant bsAb variants with reduced predicted MHC class II binding of peptides compared to bsAbs carrying S114A mutation in constant light chain and/or T187E mutation in the CH1 constant heavy chain. As reference the WT bsAb, AC_05293 was included.

FIG. 3—Melting (Tm1) and aggregation temperatures (Tagg) for the different double mutant bsAb variants. As reference the WT bsAb, AC_05293, was included.

FIG. 4—Aggregation (% HMWs) after incubation at 40° C. for 1, 2 and 4 weeks compare to before incubation. Values measured using SEC-HPLC.

FIG. 5—Freeze/thawing stability evaluation. Aggregation measured as change in % of HMWs was studied after 1 and 3 rounds of freeze/thawing.

FIG. 6—Shear stress stability evaluation for all the variants. A280 measurements were taken before incubation at 2000 rpm for 30 minutes on MixMate followed by centrifugation at 3000×g for 10 minutes before re-measuring A280.

FIG. 7—Colloidal stability measured as % PEG needed to obtain 50% protein precipitation. All variants required PEG concentrations >8-9) indicating high colloidal stability.

FIG. 8—Antigen binding of RUBY™ bsAbs, as determined by dual target ELISA. Simultaneous binding of parental bsAb AC_05332 (A), AC_05330 (B), AC_5302 (C) and mutant versions thereof to 5T4 and CD40.

FIG. 9—Binding to 5T4 expressed on CHO cells (CHO-5T4) and CHO wt cells by AC_05332 (A), AC_05330 (B), AC_5302 (C) and mutant versions thereof, as determined using flow cytometry.

FIG. 10—Binding to CD40 expressed on B16 cells (B16-CD40) and B16 wt cells by AC_05332 (A), AC_05330 (B), AC_5302 (C) and mutant versions thereof, as determined using flow cytometry.

FIG. 11—This shows an example composition of a bispecific antibody construct, which is referred to as in the RUBY™ format. The bispecific antibody of this figure is made up of three types of polypeptide chains: (1) IgG heavy chains (white) fused to Fab light chains (chequered) via a polypeptide linker. (2) IgG light chains (bricked) and (3) Fab heavy chains (black). Mutations are introduced in the interface between heavy and light chains.

FIG. 12—CD40×TAA bsAbs mediate localization of tumor debris to antigen presenting cells. The number of CEA+ tumor debris clustering with CD40+ cells was quantified after 8 hrs of culture using live cell imaging software. The graphs show the mean (+SD) of duplicate wells in one representative experiment of four (CEA).

FIG. 13—Dissociated cells from human colorectal cancer tumors were analyzed for: (left) their CEA-expression (gated on total viable cells), (middle) ability to provide cross-linking to CD40×CEA Neo-X-Prime bsAb in a CD40 reporter assay, and (right) CD83 upregulation following stimulation of the tumor infiltrating immune cells (gated on viable CD45+CD3−CD56− cells) using CD40×CEA Neo-X-Prime bsAb or isotypexCD40 bsAb (data from 1 representative experiment out of three).

FIG. 14—Simultaneous binding of CD40 and CEA by CD40×CEA bsAbs mediates activation of tonsillar cancer APCs in vitro. Human CD45+ HLA-DR+ CD3− cells from a tonsillar cancer biopsy were co-cultured with UV-irradiated CHO cells transfected with human CEA in the presence of CD40×CEA bsAb, CD40 mAb or isotype control. After 13 h culture, cells were harvested and the frequencies of CD86+ CD40+ cells were investigated using flow cytometry of CD19+ CD20+ B cells, CD14+ macrophages, CD1c+ cDC2s and XCR1+ cDC1s.

FIG. 15—MC38-CEACAM5 2 tumor growth and survival. hCD40tg mice inoculated with MC38-CEACAM5 2 tumors were dosed with the indicated treatments on days 7, 10, and 13 post-inoculation. Tumors were frequently measured, and the graphs shows the mean tumor volume (+SD) of each group until the first mouse in any of the treatment groups reached a tumor volume above the ethical limit, and the % surviving mice in each treatment group.

FIG. 16—Dissociated cells from human gastric cancer tumors were analyzed for: (left) their CEA-expression (gated on total viable cells), (middle) ability to provide cross-linking to CD40×CEA Neo-X-Prime bsAb (ffAC_05337) in a CD40 reporter assay, and (right, 1 nM CD40×CEA) CD83 upregulation following stimulation of the tumor infiltrating immune cells (gated on viable CD45+CD3−CD56− cells) using CD40×CEA Neo-X-Prime bsAb or isotypexCD40 bsAb (data from 1 representative experiment out of four).

FIG. 17—Effect of the bispecific antibody ffAC_05337on CD40 reporter cells when co-cultured with tumor cells with different CEA receptor density in the presence and absence of soluble CEA. MKN45, CEA high expressing cells (A), LS174T, CEA intermediate expressing cells (B), HT29 and LOVO, CEA low expressing cells (C-D). The response was calculated as fold induction to background.

FIG. 18—Effect of the bispecific antibody ffAC_05337 on CD40 reporter cells co-cultured with CEA expressing CHO cells and titrated antibodies in the presence or absence of soluble CEA. The response was calculated as fold induction to background.

### EXAMPLES

### Example 1—Identification of Mutations that Reduce in Silico Predicted MHC Class II Binding. Studies on Single Mutants

### Aim and Background

The objectives of this study were to identify and evaluate mutations that could remove predicted MHC class II binding of core peptides in bispecific antibodies (bsAbs) (carrying S114A mutation in constant light chain and/or T187E mutation in the CH1 constant heavy chain domain—so called ‘primary’ mutations as described herein) without negatively impacting manufacturability or target binding.

### Materials and Methods

### Core Peptide Predictions

Core peptides of selected peptides including the mutations S114A in the constant light chain and T187E in the constant heavy chain were predicted using the NetMHCIIpan 3.4 tool. The 20 most common alleles in the World Average population defined by the AbEpiAnalyzer tool (EIR Sciences) was used for predictions together with default software settings.

Design of Mutant Variants with Potentially Reduced MCH II Binding

Based on the identified core peptides a vast set of mutant peptides were designed for covering the S114A and T187E mutations. All 20 amino acids were introduced in the contact residues 1, 4, 6, and 9 of the core peptide encompassing the T187E mutation and position 1, 4 and 6 of the core peptide encompassing the S114 A mutation (S114 is in p9 of the core peptide and could therefore not be varied).

### In Silico Peptide-MHC Class II Binding Prediction Tool

The in silico predictions were performed by submitting the amino acid sequences to the AbEpiAnalyzer tool. Briefly, for each 15-mer peptide in the submitted sequence, a percentile rank is generated by comparing the predicted affinity against the affinity of a large set of 15-mers randomly selected. The rank threshold is preferred over affinity as different alleles shows biases towards high or low binding affinity. Peptides with a percentile rank <10 were included in the binding predictions, as recommended by IEDB. AbEpiAnalyzer analyses the potential immunogenicity of antibodies and sequence-modified proteins by first predicting the binding of each 15-mer peptide using the NetMHCII3.1 algorithm to a large panel of human MHC class II alleles and subsequently calculates a position-specific risk score. The score reflects the frequency of the HLA-DR, HLA-DP and HLA-DQ binding sub-peptides overlapping a given position in the defined population. The position-specific risk scores are summed over each sequence. A cutoff value of 0.1 is set for the for position-specific risk scores (i.e. all position-specific risk scores <0.1 are set to 0).

For analysis of antibodies, the program uses blastp to identify and align germline genes from a database of heavy and light V-genes. Complementary determining regions (CDRs) are identified by using a light and heavy chain hidden macro model developed based on a multiple alignment of heavy and light chains respectively. For sequence-modified proteins, the wildtype sequence is submitted separately by the user. By subtracting the HLA binding properties of the germline or wildtype protein, potential T-cell neo-epitopes are identified.

The world average population of HLA alleles was used, where the North American population has a weight of 52.7%, European 25.3%, North East Asian 9.2%, South East Asian 5.9%, South Central American 1.7%, Oceanian 1.3%, Western Asian 1.3%, Sub-Saharan African 1.0%, South Asian 0.7%, North African 0.6%, and Australian 0.1%. Results are presented as position-specific risk scores and as heat plots for identification of alleles binding and presence of high-frequency (i.e. promiscuous) neo-epitopes.

### Amino Acid Sequences

Single mutants were generated using the RUBY™ bsAb AC_05293 as the protein backbone. In addition to the potentially deimmunizing mutations, this antibody carries a set of RUBY™ specific mutations (Table 1).

TABLE 1
RUBY ™ specific mutations in AC 05293

| RUBY ™ | RUBY ™ mutations in variable | RUBY ™ mutations in constant |
|---|---|---|
| ID | domains* | domains** |
| AC_05293 | Q44R in the VH and VL of chain | H168A + F170G in CH1 of chain H1 |
| | H1 | T187E in CH1 of chain H2 |
| | Q44E in the VL of chain L1 | S114A + N137K in CKappa of chain |
| | Q44E in the VH of chain H2 | H1 |
| | | L135Y + S176W in CKappa of chain |
| | | L1 |

*According to the IMGT numbering system
**According to the EU or Kabat numbering systems

### Structural Review

3D structures of CH1 and CK of antibodies was performed using PyMOL (version 1.8.2.0) and antibody structures 5I18 and 3ZHK downloaded from the RCSB Protein Data Bank.

### Gene Synthesis and Cloning

Gene fragments incorporating the identified deimmunizing mutations of interest were obtained from Twist Bioscience and were cloned into Alligator's pCDNA3.4 expression vector together with gene fragments encoding additional parts of the bsAb AC_05293 using standard Type IIS cloning.

### Protein Production and Purification

The antibodies were expressed using transient Expi293 (Life technologies) cultures according to manufacturer's instructions. Purification of supernatants was made on Predictor MabSelectSure 50 μl 96 well plates (GE Healthcare). Cells were transfected with three different vectors encoding separately for each of the three co-expressed polypeptides chains that make up the bsAb.

Dual ELISA Plates were coated with 0.5 μg/mL CD40 in PBS over night at 4° C. After washing in PBS/0.05% Tween 20 (PBST), the plates were blocked with PBS/2% BSA for at least 30 minutes at room temperature before being washed again. Samples serially diluted in PBS/0.5% BSA were then added and allowed to bind for at least 1 hour at room temperature. After washing, plates were incubated with 0.5 μg/mL biotinylated EpCAM for at least 1 hour at room temperature. Dual complexed bsAb with CD40 and EpCAM were detected with HRP-labelled streptavidin. SuperSignal Pico Luminescent was used as substrate and luminescence signals were measured using Fluostar Optima.

### Results and Conclusions

### Identification of Core Peptides

Using in silico predictions it was possible to identify mutations that could lead to peptides with increase MHC II binding. These were peptides encompassing mutations S114A in the light chain of the appended Fab and T187E in the heavy chain of the appended Fab. Further in silico analysis could identify which of amino acids on the predicted MHC II peptides that likely are core amino acids (i.e. are involved in the interact with either the MHC II complex or the T cell receptor (TCR)) and from this the residues that are likely to interact with the MHC class II complex could be identified (position 1, 4, 6 and 9 in the core peptide). The core amino acids predicted to be in contact with the MHC class II complex are marked with an X in Table 2.

TABLE 2
Core peptide sequences for the RUBY mutations S114A and T187E

| RUBY | Sequence | |
|---|---|---|
| mutation | type | Sequence and core amino acids |
| T187E | Seq A* | AVLQSSGLXSLXSXVEXPSSSLGTQTYICNV (SEQ ID NO: 204) |
| | Seq B^{≠} | AVLQSSGLYSLSSVVTVPSSSLGTQTYICNV (SEQ ID NO: 76) |
| S114A | Seq A | TFGAGTKLEXKRXVXAPAVFIFPPSDEQL (SEQ ID NO: 205) |
| | Seq B | TFGAGTKLEIKRTVAAPSVFIFPPSDEQL (SEQ ID NO: 206) |

*Seq A sequences are the RUBY derived sequence. Underlined is the mutation needed for optimal chain pairing in RUBY. X marks the core amino acids, which are predicted to interact with the MHC II protein and can be mutated to reduce immunogenicity risks.
^{≠}Seq B are the wild-type (WT) reference sequence

The sequences of Seq A and Seq B include both the CKappa domain and the VL domain—with reference to Seq B TFGAGTKLEIKRTVAAPSVFIFPPSDEQL (SEQ ID NO: 206), the CKappa domain is underlined and VL emboldened.

### Identification of Deimmunized RUBY Variants

Using the predicted core amino acids, potential mutants with lower predicted MHC class II binding were evaluated using in silico MHC class II peptide binding analysis. The results revealed several mutations that reduced the predicted risk score to or close to 0 of the S114A mutation in the light chain of the appended Fab and T187E in the heavy chain of the appended Fab.

A detailed review of each identified mutation with potential to reduce the immunogenicity of RUBY was carried out to remove mutations that would introduce amino acids or sites with unfavourable manufacturability characteristics as well as mutations that based on their location in the 3D structure were very unlikely to generate functional and stable proteins. The full list of remaining deimmunizing mutations that were selected for further evaluation can be found in Table 3.

TABLE 3
Sequence ID and mutations selected for
protein production and evaluations.

| | SEQ_ID | Mutations |
|---|---|---|
| Mutations to | 4109 | I126A** in VL |
| reduce predicted | 4110 | I126G** in VL |
| MHCII binding | 4111 | I126H** in VL |
| caused by S114A* | 4112 | I126N** in VL |
| on the CKappa | 4113 | I126P** in VL |
| light chain | 4114 | I126Q** in VL |
| | 4115 | I126S** in VL |
| | 4116 | I126T** in VL |
| | 4117 | T109P* in CKappa |
| | 4118 | A111R* CKappa |
| | 4119 | A111T* CKappa |
| | 4120 | A111V* CKappa |
| | 4121 | A111W* CKappa |
| Mutations to | 4124 | Y180A* CKappa |
| reduce predicted | 4125 | Y180G* CKappa |
| MHCII binding | 4126 | Y180I* CKappa |
| caused by T187E* | 4127 | Y180N* CKappa |
| on CH1 | 4128 | Y180S* CKappa |
| | 4129 | Y180T* CKappa |
| | 4130 | Y180V* CKappa |
| | 4131 | Y180W* CKappa |
| | 4132 | S183N* CKappa |
| | 4133 | S183T* CKappa |
| | 4134 | V188G* CKappa |

*According to the EU or Kabat numbering systems
**According to the IMGT numbering system

### Evaluation of Single Site Mutants

Cloning and Production RUBY variants carrying one of the additional mutations listed in Table 3 were engineered and produced in high throughput 96-well format (HT production). Productivity from transient cultures of the mutant variants were in general good and at levels comparable to the wild-type bsAb (data not shown).

### Dual ELISA

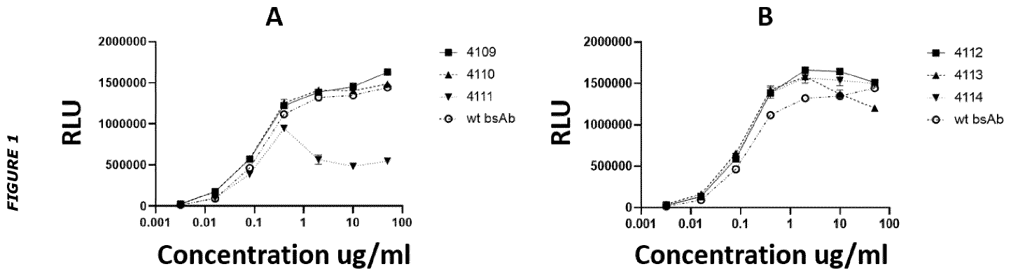

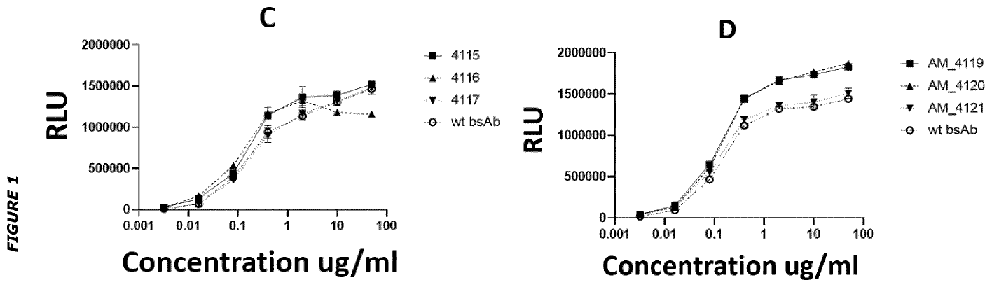

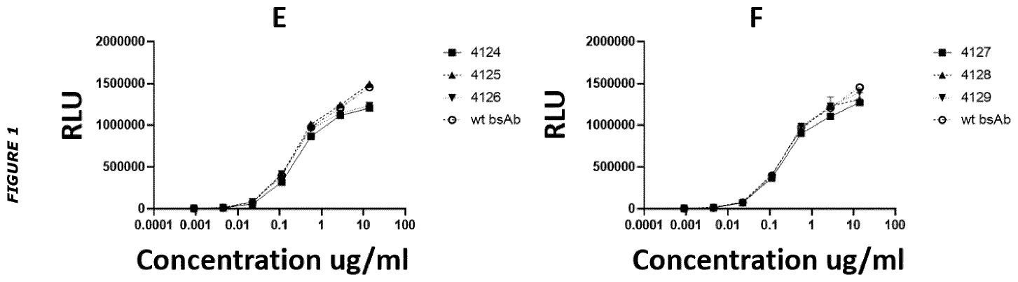

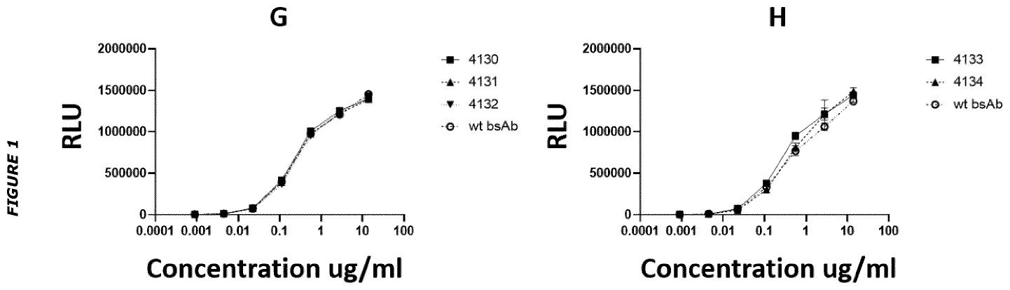

Simultaneous binding to both targets (CD40 and EpCAM) was evaluated using a dual ELISA setup. As can be seen in FIG. 1, a vast majority of the evaluated mutants retained binding to both targets at levels comparable to the wild-type bsAb. The results from the dual ELISA were further confirmed by mono ELISAs against both targets (data not shown).

In all, it could be concluded that a diverse set of single amino acid mutations that reduced the in silico predicted immunogenicity risk of RUBY™ bsAbs derived peptides carrying mutations S114A or T187E without negatively impacting manufacturability or target binding could be identified.

### Example 2—Evaluation of Mutation Combinations that Reduce in Silico Predicted MHC Class II Binding—Studies on Double Mutants

### Aim and Background

The objectives of this study were to evaluate combination of mutations that reduce the in silico predicted MHC class II binding and a identify pair of mutations that could reduce predicted MHC class II binding of core peptides in RUBY™ bsAbs (carrying S114A mutation in constant light chain domain and/or T187E mutation in the CH1 constant heavy chain domain) without negatively impacting antigen binding, stability or developability.

### Materials and Methods

### Structural Review

3D structures of CH1 and CK of antibodies was performed using PyMOL (version 1.8.2.0) and antibody structures 5118 and 3ZHK downloaded from the RCSB Protein Data Bank.

### Amino Acid Sequences

Double mutants were generated using AC_05293 bsAb as the protein backbone. In addition to the potentially deimmunizing mutations, this antibody carries a set of RUBY™ specific mutations (Table 4). All 20 possible double mutant combinations of Set A and Set B mutations listed in Table 5 were generated.

TABLE 4
RUBY ™ specific mutations in AC 05293

| RUBY ™ | RUBY ™ mutations in variable | RUBY ™ mutations in constant |
|---|---|---|
| ID | domains* | domains** |
| AC_05293 | Q44R in the VH and VL of chain | H168A + F170G in CH1 of chain H1 |
| | H1 | T187E in CH1 of chain H2 |
| | Q44E in the VL of chain L1 | S114A + N137K in CKappa of chain |
| | Q44E in the VH of chain H2 | H1 |
| | | L135Y + S176W in CKappa of chain |
| | | L1 |

*According to the IMGT numbering system
**According to the EU or Kabat numbering systems

### Gene Synthesis and Cloning

Gene fragments incorporating the identified deimmunizing mutations of interest were obtained from Twist Bioscience and were cloned into Alligator's pCDNA3.4 expression vector together with gene fragments encoding additional parts of the bsAb AC_05293 using standard Type IIS cloning.

### Protein Production and Purification

The antibodies were expressed using transient Expi293 (Life technologies) cultures at different volumes (high throughput 1.2 mL or 30 mL) according to manufacturer's instructions. Purification of supernatants from 30 mL cultures was made on protein A using the NGC system (BioRad) or Predictor MabSelectSure 50 μl 96 well plates (GE Healthcare) for supernatants from high throughput expressions. Cells were transfected with three different vectors encoding separately for each of the three co-expressed polypeptides chains that make up the bsAb.

### SEC-HPLC

Aggregation was measured with SE-HPLC in a 1260 Infinity II system (Agilent Technologies) using a TSK gel Super SW mAB HTP 4 μm, 4.6×150 mm column (TOSOH Bioscience) and 100 mM Sodium Phosphate, pH 6.8, 300 mM NaCl as mobile phase at ambient temperature and a flow rate of 0.35 ml/min.

### LabCHIP

Analysis of all samples was performed on the LabChip GXII (PerkinElmer) as described by the manufacturer and following our routine guideline DOCID-371837740-15. Briefly, 2 μL of each protein sample (reduced or non-reduced) were denatured at 90° C. for 5 minutes, centrifuged and loaded into the microfluidic chip.

### Mono ELISA

Plates were coated with 0.5 μg/mL of either CD40 or EpCAM in PBS over night at 4° C. After washing in PBS/0.05% Tween 20 (PBST), the plates were blocked with PBS/2% BSA for at least 30 minutes at room temperature before being washed again. Samples serially diluted in PBS/0.5% BSA were then added and allowed to bind for at least 1 hour at room temperature. After washing, plates were incubated with a secondary anti kappa antibody for at least 1 hour at room temperature. After washing, SuperSignal Pico Luminescent was used as substrate and luminescence signals were measured using Fluostar Optima.

### Dual ELISA

Plates were coated with 0.5 μg/mL CD40 in PBS over night at 4° C. After washing in PBS/0.05% Tween 20 (PBST), the plates were blocked with PBS/2% BSA for at least 30 minutes at room temperature before being washed again. Samples serially diluted in PBS/0.5% BSA were then added and allowed to bind for at least 1 hour at room temperature. After washing, plates were incubated with 0.5 μg/mL biotinylated EpCAM for at least 1 hour at room temperature. Dual complexed bsAb with CD40 and EpCAM were detected with HRP-labelled streptavidin. SuperSignal Pico Luminescent was used as substrate and luminescence signals were measured using Fluostar Optima.

### Octet

Kinetic measurements were performed using the Octet RED96 platform (ForteBIo). Biotinylated antigens were coupled to Streptavidin sensors (ForteBio) at antigen concentrations of 0.25 μg/mL CD40 or 0.5 μg/mL EpCAM. Antibodies were serially diluted ½ in 1× Kinetic buffer (ForteBio) with start concentrations of 10 nM CD40 or 25 nM EpCAM and analysed for binding to antigen-coupled sensors. Association was followed for 300 seconds followed by dissociation in 1x Kinetic buffer for 300 seconds. Sensor tips were regenerated using 10 mM glycine, pH 1.7. Data generated were referenced by subtracting a parallel buffer blank, the baseline was aligned with the γ-axis, inter-step correlation by alignment against dissociation was performed and the data were smoothed by a Savitzky-Golay filter in the data analysis software (v.9.0.0.14). The processed data were fitted using a 1:1 Langmuir binding model with X2 as a measurement of fitting accuracy.

### Thermostability

The melting temperatures (Tm1) and aggregation temperature (Tagg) were measured with the UNCle system (UNchained labs). The intrinsic fluorescence was measured during linear temperature ramping from 20° C. to 95° C. at a rate of 0.4° C./minute. The data analysis was performed with the UNcle Analysis software version 2.0 using default settings.

### Stability

Samples in non-optimized buffer (PBS) were incubated at 2-8° C. and 40° C. for 1, 2 and 4 weeks or subjected to 3 rounds (24, 48 and 72 hours) of freeze thawing. Visual inspection was performed and protein degradation was measured with SE-HPLC, LabChip and dual ELISA.

### Shear Stress Stability

Samples in triplicates were subjected to shear stress in form of heavy agitation at 2000 rpm on the 96-well plate shaker MixMate (Eppendorf) for 30 minutes. Protein precipitates were removed by centrifugation at 3000 g for 10 minutes and the absorbance at 280 nm was measured using BigLunatic.

### Colloidal Stability

Samples were mixed with PBS/PEG solutions with different PEG concentrations ranging from 4%-36%, added to 96-well filter plates in duplicates and incubated over night at room temperature. Filtrates obtained by centrifugation at 12000 g for 15 minutes were collected and spun down before the absorbance at 280 nm was measured using BigLunatic (to determine loss of protein) and compared to controls.

### Results and Conclusions

Selection of Mutation to Combine into Novel Deimmunized RUBY Variants

Based on the very promising results obtained from the production and ELISA evaluation of the single site mutants, a set of promising mutations for each risk site was selected after review of the 3D structure surrounding these sites. The biophysical properties, such as hydrophobicity, charge and size, together with the likelihood of beneficial/detrimental interactions with surrounding amino acid side chains was considered and the selected sets of mutants are listed in Table 5.

TABLE 5
Sequence ID and mutations selected for
protein production and evaluations.

| | | ID No. | Mutations |
|---|---|---|---|
| Set A | Mutations to | 4109 | I126A** |
| | reduce predicted | 4110 | I126G** |
| | MHCII binding | 4115 | I126S** |
| | caused by S114A* | | |
| | on the CKappa | 4117 | T109P* |
| | light chain | 4120 | A111V* |
| Set B | Mutations to | 4126 | Y180I* |
| | reduce predicted | | |
| | MHCII binding | 4131 | Y180W* |
| | caused by T187E* | 4133 | S183T* |
| | on CH1 | 4134 | V188G* |

*According to the EU or Kabat numbering systems
** According to the IMGT numbering system

### Manufacturability

BsAb double mutants were produced from transient cultures. Expression was carried out in 30 mL cultures of Expi293. Expression yields were calculated using A280 measurements determined by BigLunatic after MabSelectSure purification. SEC-HPLC analysis was performed to ascertain protein size, aggregation and fragmentation. In addition, LabCHIP (reduced and un-reduced conditions) was performed to further assess aggregation and fragmentation. 16 of the 20 variants had yields above 100 mg/L (Table 6) and purity above 98%. Overall, good yields and purity were obtained comparable to the wild type bsAb.

TABLE 6
Expression yields, and SEC-HPLC data for double bsAb mutants.

| | | | Main | |
|---|---|---|---|---|
| | Yield | HMWS* | peak | LMWS ** |
| BsAb variant | (mg/L) | (%) | area (%) | (%) |
| AM_4109-4126 | 68 | 2 | 98 | 0.1 |
| AM_4109-4131 | 121 | 2 | 98 | 0.2 |
| AM_4109-4133 | 148 | 1 | 99 | 0.1 |
| AM_4109-4134 | 123 | 2 | 98 | 0.2 |
| AM_4110-4126 | 118 | 2 | 98 | 0.2 |
| AM_4110-4131 | 136 | 2 | 97 | 0.2 |
| AM_4110-4133 | 125 | 2 | 98 | 0.1 |
| AM_4110-4134 | 93 | 3 | 97 | 0.2 |
| AM_4115-4126 | 73 | 5 | 95 | 0.2 |
| AM_4115-4131 | 79 | 5 | 95 | 0.2 |
| AM_4115-4133 | 128 | 2 | 98 | 0.1 |
| AM_4115-4134 | 110 | 3 | 97 | 0.1 |
| AM_4117-4126 | 160 | 2 | 98 | 0.1 |
| AM_4117-4131 | 152 | 2 | 98 | 0.1 |
| AM_4117-4133 | 155 | 1 | 99 | 0.1 |
| AM_4117-4134 | 155 | 2 | 98 | 0.1 |
| AM_4120-4126 | 147 | 1 | 99 | 0.1 |
| AM_4120-4131 | 141 | 1 | 99 | 0.1 |
| AM_4120-4133 | 153 | 1 | 99 | 0.1 |
| AM_4120-4134 | 120 | 2 | 98 | 0.1 |
| wt bsAb (AC_05293) | 159 | 1 | 99 | 0.1 |

*High molecular weight species
**Low molecular weight species

### Dual ELISA

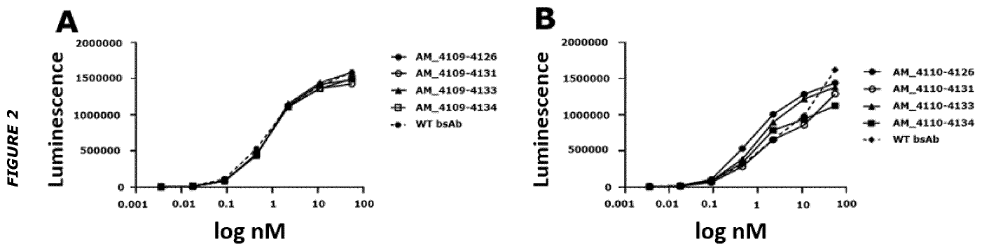

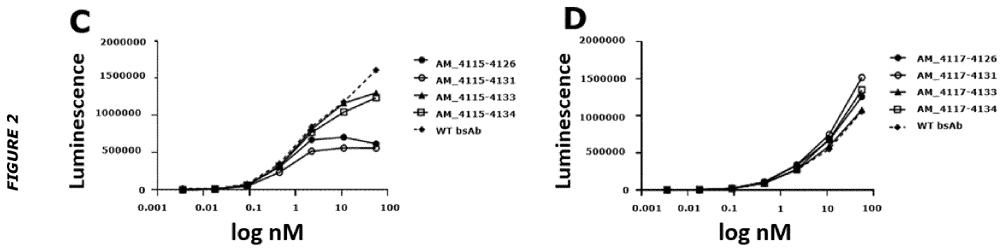

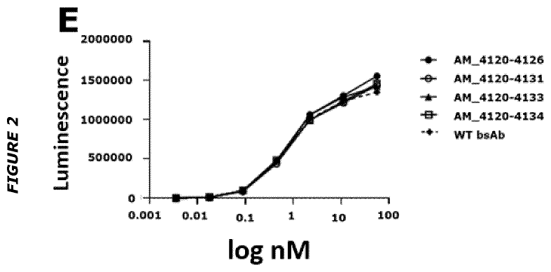

Mono and dual ELISAs were performed to assess binding to targets of the different double mutant variants. Mono ELISA data showed good binding for all the variants to their single targets (CD40 or EpCAM) (data not shown). A similar trend was observed with dual ELISA except for variants AM_4115-4126 and AM_4115-4131 (FIG. 2) that showed reduced binding.

### Octet

Binding affinities of all the variants were determined using Octet (Pall ForteBio). Biotinylated antigens (CD40 or EpCAM) were immobilized on Streptavidin biosensors and binding to variants and wild type bsAb in solution was measured. Similar KID was observed for all variants and wild type bsAb binding to CD40. A similar trend was displayed when binding to EpCAM except for all mutants carrying the 8126S mutation (ID No. 4115) that showed slightly higher KID (Table 7). The reduction in EpCAM affinity for I126S mutants is in compliance with the results obtained in ELISA (FIG. 2).

TABLE 7
Binding affinity measurements by Octet.

| | CD40 | | | EpCAM | | |
|---|---|---|---|---|---|---|
| BsAb variant | KD (M) | kon(1/Ms) | koff(1/s) | KD (M) | kon(1/Ms) | koff(1/s) |
| AM_4109-4126 | 1.2E−09 | 4.8E+05 | 6.0E−04 | 4.3E−09 | 1.8E+05 | 7.7E−04 |
| AM_4109-4131 | 8.9E−10 | 8.0E+05 | 7.2E−04 | 4.4E−09 | 1.8E+05 | 8.1E−04 |
| AM_4109-4133 | 7.3E−10 | 1.4E+06 | 1.0E−03 | 4.6E−09 | 2.0E+05 | 9.2E−04 |
| AM_4109-4134 | 8.3E−10 | 1.1E+06 | 9.2E−04 | 4.7E−09 | 2.1E+05 | 9.8E−04 |
| AM_4110-4126 | 1.5E−09 | 3.5E+05 | 5.1E−04 | 5.1E−09 | 1.7E+05 | 9.0E−04 |
| AM_4110-4131 | 1.2E−09 | 4.5E+05 | 5.6E−04 | 4.7E−09 | 2.0E+05 | 9.5E−04 |
| AM_4110-4133 | 1.1E−09 | 6.8E+05 | 7.5E−04 | 4.9E−09 | 1.9E+05 | 9.6E−04 |
| AM_4110-4134 | 8.8E−10 | 8.6E+05 | 7.5E−04 | 5.3E−09 | 2.0E+05 | 1.1E−03 |
| AM_4115-4126 | 1.1E−09 | 7.4E+05 | 8.5E−04 | 6.9E−09 | 1.7E+05 | 1.1E−03 |
| AM_4115-4131 | 9.7E−10 | 9.2E+05 | 8.9E−04 | 7.2E−09 | 1.8E+05 | 1.3E−03 |
| AM_4115-4133 | 1.0E−09 | 1.1E+06 | 1.1E−03 | 7.0E−09 | 1.8E+05 | 1.3E−03 |
| AM_4115-4134 | 1.1E−09 | 9.3E+05 | 1.0E−03 | 6.9E−09 | 1.9E+05 | 1.3E−03 |
| AM_4117-4126 | 1.2E−09 | 4.0E+05 | 4.8E−04 | 4.6E−09 | 2.2E+05 | 1.0E−03 |
| AM_4117-4131 | 1.0E−09 | 6.3E+05 | 6.6E−04 | 4.5E−09 | 2.6E+05 | 1.2E−03 |
| AM_4117-4133 | 9.3E−10 | 7.5E+05 | 7.0E−04 | 4.6E−09 | 2.6E+05 | 1.2E−03 |
| AM_4117-4134 | 9.7E−10 | 7.4E+05 | 7.2E−04 | 5.2E−09 | 2.3E+05 | 1.2E−03 |
| AM_4120-4126 | 8.6E−10 | 1.2E+06 | 1.0E−03 | 5.6E−09 | 2.3E+05 | 1.3E−03 |
| AM_4120-4131 | 1.1E−09 | 9.2E+05 | 9.7E−04 | 5.3E−09 | 2.8E+05 | 1.5E−03 |
| AM_4120-4133 | 9.0E−10 | 1.2E+06 | 1.1E−03 | 5.7E−09 | 2.4E+05 | 1.4E−03 |
| AM_4120-4134 | 9.1E−10 | 1.2E+06 | 1.1E−03 | 5.8E−09 | 2.5E+05 | 1.4E−03 |
| wt bsAb | 9.3E−10 | 1.1E+06 | 1.0E−03 | 4.8E−09 | 2.3E+05 | 1.2E−03 |
| (AC_05293) | | | | | | |

### Stability

To assess the stability of the variants, melting temperature (Tm), shear stress, colloidal stability and storage stability at different temperatures were evaluated.

### Thermostability

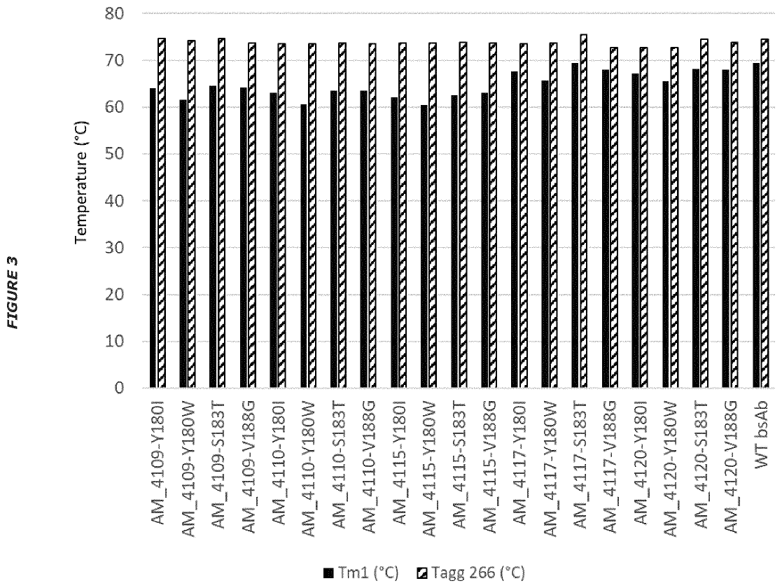

Analysis was performed using UNcle instrument (UNchained Biolabs) to determine the variants' melting (Tint) and aggregation temperatures (Tagg). Variants carrying mutations I126A (4109), I126G (4110) and I126S (4115) had slightly lowered Tint (<65° C.), as compared to the wt bsAb, while T109P (4117) and A111V (4120) variants had Tint >65° C. (FIG. 3). The aggregation temperatures on the other hand were very similar for all bsAb double mutants and comparable to the WT bsAb, i.e. around 75° C., ±1-2° C.

### Elevated Temperature and Freeze/Thawing Stability

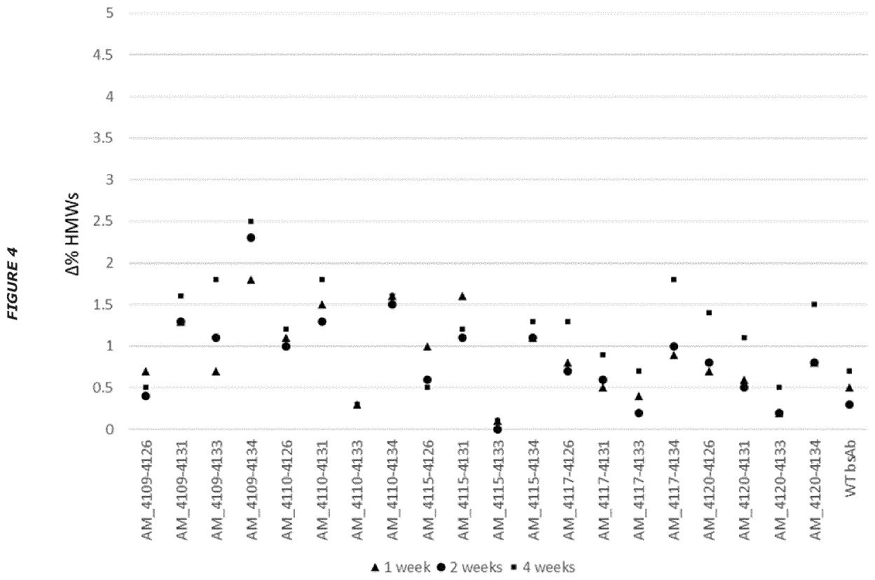

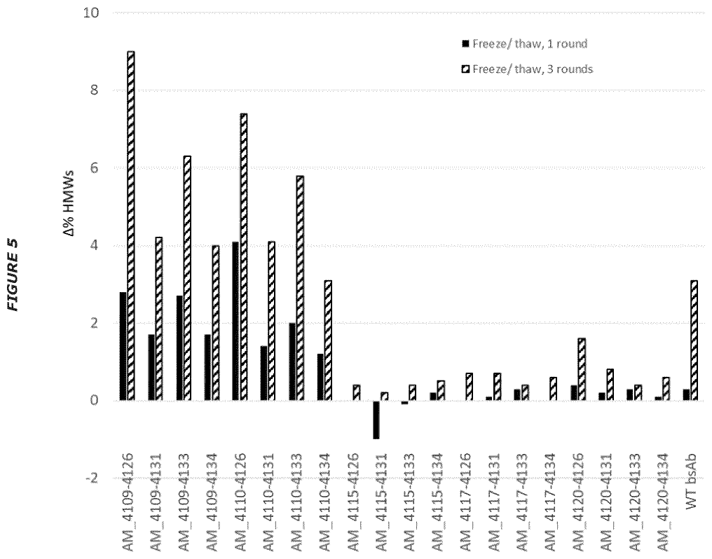

Stability at elevated temperature was performed by incubating the proteins at 40° C. for up to 4 weeks. LabCHIP and dual ELISA analyses were performed for all samples after 4 weeks incubation while SEC-HPLC analyses were performed for samples at weeks 1, 2 and 4. Freeze-thawing stability was evaluated by incubating proteins at −80° C. and thawing them in a water bath set at 37° C. after 24, 48 and 72 hours. SEC-HPLC analysis was performed only on samples thawed after 24 and 72 hours. In addition, all samples were visually inspected for any particles at each of the three-time points. Minimal aggregation (<3% change in HMWs) was observed for all the variants after storage at 40° C. even after 4 weeks incubation indicating very high temperature stability for all double mutants (FIG. 4). Slight aggregation after three rounds of freeze/thawing was observed for I126A (4109) and I126G (4110) compared to the WT bsAb. Surprisingly all mutants carrying I126S (4115), T109P (4117) and A111V (4120) showed an improved freeze-thawing compared to the WT bsAb (FIG. 5) indicating that the mutations not only lead to potential decreased predicted MHCII binding but also improved stability.

### Shear Stress

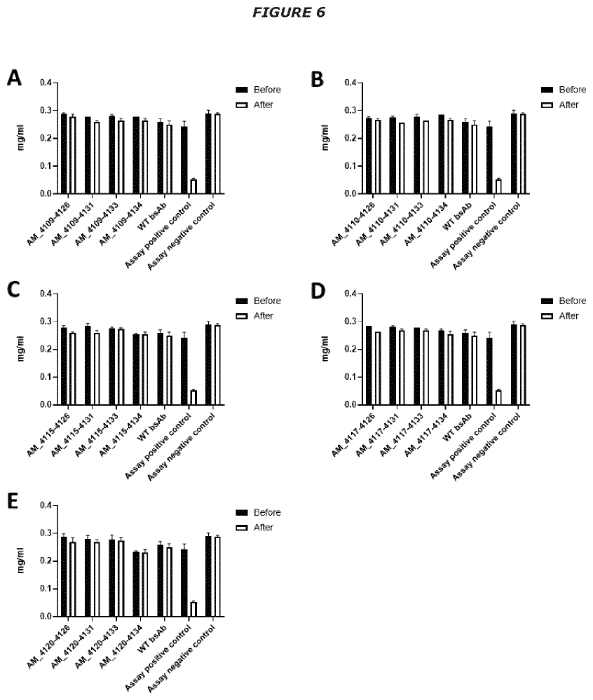

Evaluation of the ability of all the variants to withstand degradation due to shear stress was performed using Alligator's in-house developed shear stress assay. The assay is based on agitation at a given shaking speed and time. The amount of protein loss is determined by calculating the changes in A280 before and after agitation. All the variants were shown to be highly shear stress stable (FIG. 6) since the differences between protein concentration before after agitation were small. Overall, less than 10% protein loss due to agitation was registered for all the constructs.

### Colloidal Stability

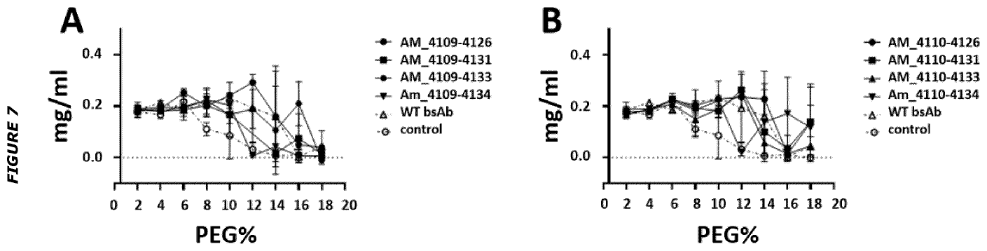

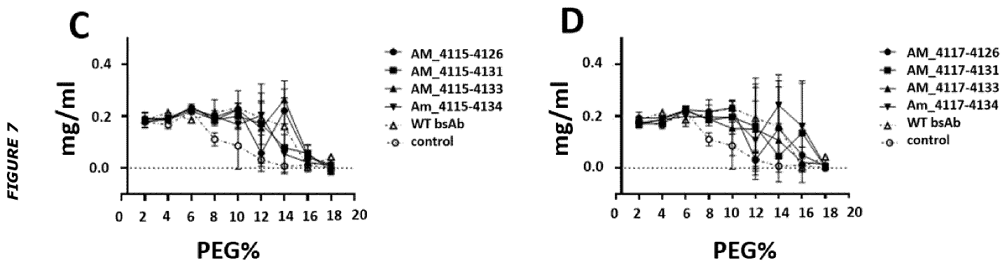

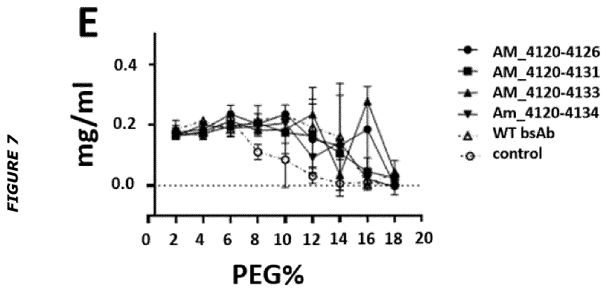

The variants were further assessed for colloidal stability using a PEG precipitation assay. Addition of PEG at different concentrations to the protein solution leads to precipitation as a result of exclusion volume effects. PEG being a long-chain polymer occupies more space in the solution than a protein of a similar molecular mass and this lowers protein solubility resulting into protein precipitation. The assay gives a relative measurement of the colloidal stability measured as the concentration of PEG needed to precipitate 50% of the protein amount in a sample. The higher the PEG concentration needed the higher the colloidal stability. Values are compared to a control, that is considered to have high colloidal stability and for which it is known that 8-9% PEG is required to obtain 50% protein precipitation. All the double mutant variants showed colloidal stability comparable to the WT bsAb and performed better than the control (FIG. 7).

All in all, several of the tested double variants displayed in general good binding, stability and manufacturability in transient cultures. Mutations I126S, T109P, A111V in the constant light chain and mutation S183T and V188G in the constant heavy chain domain CH1 were particularly beneficial in terms of stability and combination of mutations T109P, A111V in the constant light chain and mutation S183T and V188G in the constant heavy chain domain CH1 resulted in an overall good target binding, high thermostability, colloidal stability and general high stress stability (including stresses such as elevated temperatures, freeze/thawing and shear stress).

### Example 3—Identification of MHC Class II Binding Peptides Using MHC Associated Peptide Proteomics (MAPPs) Assay

### Aim

The aim of the current study was to identify non-self peptides derived from RUBY™ bispecific antibodies (bsAbs), with or without optimizing mutations S183T in the CH1 of chain H2 and T109P in the CKappa of chain H1, that are naturally processed and presented on MHC class II by antigen presenting cells. Such non-self peptides that are displayed on MHC class II have the potential to provoke immunogenic responses and induction of undesired anti-drug antibodies. A reduced number of such peptides in a protein drug, such as a bispecific antibody, is thus beneficial and could translate into a better safety profile in clinical settings.

### Materials & Methods

The two RUBY™ bsAbs included in the assay and the set of optimizing mutations included are described in Table 8. In addition to the mutations listed in Table 8, both RUBY™ bsAbs carry mutations Q44R in the variable heavy (VH) domain and variable light (VL) domain of chain H1, Q44E in the VL domain of chain L1 and Q44E in the VH of chain H2. However, only RUBY™ specific mutations incorporated in constant domains of the bsAbs are taken into consideration in this evaluation as any peptides encompassing mutations the variable domains are likely to be affected by the specific amino acid sequence of that specific variable domain and not an intrinsic feature of the RUBY™ bsAb format.

TABLE 8
Summary of mutations in constant domains of RUBY ™ bsAbs
evaluated in MAPPs assay

| RUBY ™ | RUBY ™ mutations in constant | Additional optimizing |
|---|---|---|
| ID | domains | mutations |
| AC_05293 | H168A + F170G in CH1 of chain H1 | — |
| Multi46 | T187E in CH1 of chain H2 | S183T in the CH1 of chain H2 |
| | S114A + N137K in CKappa of chain | T109P in the CKappa of chain |
| | H1 | H1 |
| | L135Y + S176W in CKappa of chain | |
| | L1 | |

In brief, Monocyte-de rived Dendritic cells (MoDCs) were derived from a cohort of 10 healthy donors (HLA typing available in Table 8) and incubated with the test samples. MoDCs were then lysed, HLA-DR/peptide complexes immunocaptured, peptides eluted and sequenced by Mass Spectrometry (MS). MS data analysis identified peptides unique to individual donors and common across multiple donors. The identified peptides were further analyzed to filter out self-peptides sequences from the final data set. Self-peptide sequences are considered unlikely to contain T cell epitopes capable of inducing anti-drug antibody (ADA) responses, due to either thymic selection or peripheral T cell tolerance.

TABLE 9
HLA haplotypes identified in donors included in MAPPs assay.

| Donor | Haplotype | | | | | | | | | |
|---|---|---|---|---|---|---|---|---|---|---|
| no | DRB1 | DRB1 | DRB2 | DRB3 | DRB4 | DRB5 | DQA1 | DQA1 | DQB1 | DQB1 |
| 1 | 04:05 | 07:01 | | | 01:03 | | 02:01 | 03:03 | 02:02 | 03:02 |
| 2 | 01:01 | 04:01 | | | 01:03 | | 01:01 | 03:03 | 03:01 | 05:01 |
| 3 | 07:01 | 15:02 | | | 01:03 | 01:02 | 01:03 | 02:01 | 02:02 | 06:01 |
| 4 | 03:01 | 13:02 | 01:01 | 03:01 | | | 01:02 | | | |
| 5 | 04:01 | 11:01 | 02:02 | | 01:03 | | 03:03 | 05:05 | 03:01 | |
| 6 | 11:01 | | 02:02 | | | | 05:05 | | 03:01 | |
| 7 | 04:07 | 08:01 | | | 01:03 | | 03:03 | 04:01 | 03:01 | 04:02 |
| 8 | 03:01 | 04:01 | 01:01 | | 01:03 | | 03:01 | 05:01 | 02:01 | 03:02 |
| 9 | 09:01 | 13:01 | 02:02 | | 01:03 | | 01:03 | 03:02 | 03:03 | 06:03 |
| 10 | 03:01 | 11:04 | 02:02 | | | | 05:01 | 05:05 | 02:01 | 03:01 |

In addition, an in silico analysis of all identified non-self peptides were performed using the iTope-AI algorithm (Abzena Ltd), were binding to the 46 most common MHC class II alleles is predicted. A “Position Risk Score” is calculated, representing the sum of predicted binding to each allotype.

### Results and Conclusions

Results from the MAPPs assay and in silico prediction for AC_05293 and Multi46 are summarized in Table 10 and Table 11. In general, the number of identified peptides created by RUBY™ specific mutations was low and the frequency of donors presenting such peptides no more than 30%. For AC_05293, no peptides encompassing mutations H168A or H170G in the CH1 of the H1 chain or mutation L135Y in the CKappa of the L1 chain was identified in any of the donors. Varying numbers of peptides containing mutations S114A and N137L in the CKappa of chain H1, T187E in the CH1 of H2 as well as S176W in the CKappa of L1 was identified in 1 to 3 donors and the total Position Risk Score of these peptides ranged from 4 to 81 (summed bsAb Position Risk Score=122). No new peptide clusters were identified in the analysis of Multi46, but with the introduction of optimizing mutations T109P in the CKappa of chain H1 and S183T in the CH1 of chain H2 both the number of identified peptides and total Position Risk Scores were reduced. After introduction of optimizing mutation T109P no peptides covering RUBY™ specific mutation S114A in the CKappa of H1 was identified in any of the 10 donors, compared to the 2 peptides covering this mutation identified in two donors in the analysis of AC_05293. Although the introduction of optimizing mutation S183T did not reduce the number of peptides presented covering mutation T187E in the CH1 of chain H2, the frequency of donors presenting such peptides was reduced from 3 to 2 and the total Position Risk Score for these peptides was reduced from 12 to 8. In total, the summed bsAb Position Risk score was dramatically decreased from 122 to 37 by the introduction of the two optimizing mutations to the CKappa of the H1 chain and CH1 of the H2 chain.

TABLE 10
Summary of results from MAPPs and in silico analysis of AC_05293

| | | | | | Total |
|---|---|---|---|---|---|
| | | | Donor(s) | | Position |
| | | | presenting | Frequency | Risk |
| Chain | Mutation | Peptide sequence | peptide | (%) | Score |
| H1 | H168A | — | — | — | |
| | (CH1) | | | | |
| | F170G | — | — | — | |
| | (CH1) | | | | |
| | S114A | TKLEIKRTVAAPAVFI (SEQ | 2, 4 | 20 | 81 |
| | (CKappa) | ID NO: 207) | | | |
| | | TKLEIKRTVAAPAVFIFPP | 4 | | |
| | | (SEQ ID NO: 208) | | | |
| | N137L | SGTASVVCLLKNFYPREA | 10 | 10 | 4 |
| | (CKappa) | (SEQ ID NO: 209) | | | |
| | | GTASVVCLLKNFYPR (SEQ ID | 10 | | |
| | | NO: 210) | | | |
| | | TASVVCLLKNFYPR (SEQ ID | 10 | | |
| | | NO: 211) | | | |
| H2 | T187E | SSGLYSLSSVVEVPS (SEQ ID | 2, 4, 9 | 30 | 12 |
| | (CH1) | NO: 212) | | | |
| | | SSGLYSLSSVVEVPSS (SEQ | 2, 4, 9 | | |
| | | ID NO: 213) | | | |
| | | SGLYSLSSVVEVPS (SEQ ID | 2, 4, 9 | | |
| | | NO: 214) | | | |
| | | SGLYSLSSVVEVPSS (SEQ ID | 2, 4 | | |
| | | NO: 215) | | | |
| L1 | L135Y | — | — | — | |
| | (CKappa) | | | | |
| | S176W | EQDSKDSTYSLWSTLTLSK | 2 | 20 | 25 |
| | (CKappa) | (SEQ ID NO: 216) | | | |
| | | EQDSKDSTYSLWSTLTLSKA | 2 | | |
| | | (SEQ ID NO: 217) | | | |
| | | EQDSKDSTYSLWSTLTLSKAD | 2 | | |
| | | (SEQ ID NO: 218) | | | |
| | | QDSKDSTYSLWSTLTLSKA | 2 | | |
| | | (SEQ ID NO: 219) | | | |
| | | DSKDSTYSLWSTLTLSK | 2 | | |
| | | (SEQ ID NO: 220) | | | |
| | | DSKDSTYSLWSTLTLSKA | 2, 3 | | |
| | | (SEQ ID NO: 221) | | | |
| | | DSKDSTYSLWSTLTLSKAD | 3 | | |
| | | (SEQ ID NO: 222) | | | |
| | | DSKDSTYSLWSTLTLSKADY | 3 | | |
| | | (SEQ ID NO: 223) | | | |
| | | SKDSTYSLWSTLTLSKA (SEQ | 2 | | |
| | | ID NO: 224) | | | |
| | | SKDSTYSLWSTLTLSKAD | 3 | | |
| | | (SEQ ID NO: 225) | | | |
| | | KDSTYSLWSTLTL (SEQ ID | 3 | | |
| | | NO: 226) | | | |
| | | KDSTYSLWSTLTLSK (SEQ | 2, 3 | | |
| | | ID NO: 227) | | | |
| | | KDSTYSLWSTLTLSKA (SEQ | 2, 3 | | |
| | | ID NO: 228) | | | |
| | | KDSTYSLWSTLTLSKAD | 3 | | |
| | | (SEQ ID NO: 229) | | | |
| | | KDSTYSLWSTLTLSKADY | 3 | | |
| | | (SEQ ID NO: 230) | | | |
| | | DSTYSLWSTLTLSK (SEQ ID | 2, 3 | | |
| | | NO: 231) | | | |
| | | DSTYSLWSTLTLSKA (SEQ | 2, 3 | | |
| | | ID NO: 232) | | | |
| | | DSTYSLWSTLTLSKAD (SEQ | 3 | | |
| | | ID NO: 233) | | | |

TABLE 11
Summary of results from MAPPs and in silico analysis of Multi46

| | Mutation | | | | Total |
|---|---|---|---|---|---|
| | (Optimizing | | Donor(s) | | Position |
| | mutations | | presenting | Frequency | Risk |
| Chain | in bold) | Peptide sequence | peptide | (%) | Score |
| H1 | H168A | — | — | — | |
| | (CH1) | | | | |
| | F170G | — | — | — | |
| | (CH1) | | | | |
| | T109P + | — | — | — | |
| | S114A | | | | |
| | (CKappa) | | | | |
| | N137L | SGTASVVCLLKNFYPR (SEQ ID | 10 | 20 | 4 |
| | (CKappa) | NO: 234) | | | |
| | | SGTASVVCLLKNFYPRE (SEQ | 10 | | |
| | | ID NO: 235) | | | |
| | | SGTASVVCLLKNFYPREA (SEQ | 10 | | |
| | | ID NO: 236) | | | |
| | | SGTASVVCLLKNFYPREAKVQW | 3 | | |
| | | KV (SEQ ID NO: 237) | | | |
| | | GTASVVCLLKNFYPR (SEQ ID | 10 | | |
| | | NO: 238) | | | |
| | | TASVVCLLKNFYPR (SEQ ID | 10 | | |
| | | NO: 239) | | | |
| H2 | S183T + | SSGLYSLSSVVEVPS (SEQ ID | 2, 4 | 20 | 8 |
| | T187E | NO: 240) | | | |
| | (CH1) | SSGLYSLSSVVEVPSS (SEQ ID | 2, 4 | | |
| | | NO: 241) | | | |
| | | SSGLYSLSSVVEVPSSS (SEQ | 4 | | |
| | | ID NO: 242) | | | |
| | | SGLYSLSSVVEVPS (SEQ ID | 2, 4 | | |
| | | NO: 243) | | | |
| L1 | L135Y | — | — | — | |
| | (CKappa) | | | | |
| | S176W | EQDSKDSTYSLWSTLTLSK | 2 | 20 | 25 |
| | (CKappa) | (SEQ ID NO: 244) | | | |
| | | EQDSKDSTYSLWSTLTLSKA | 2 | | |
| | | (SEQ ID NO: 245) | | | |
| | | EQDSKDSTYSLWSTLTLSKAD | 2 | | |
| | | (SEQ ID NO: 246) | | | |
| | | DSKDSTYSLWSTLTLSK (SEQ | 2 | | |
| | | ID NO: 247) | | | |
| | | DSKDSTYSLWSTLTLSKA (SEQ | 2, 3 | | |
| | | ID NO: 248) | | | |
| | | DSKDSTYSLWSTLTLSKAS | 3 | | |
| | | (SEQ ID NO: 249) | | | |
| | | DSKDSTYSLWSTLTLSKADY | 3 | | |
| | | (SEQ ID NO: 250) | | | |
| | | SKDSTYSLWSTLTLSKA (SEQ | 2 | | |
| | | ID NO: 251) | | | |
| | | SKDSTYSLWSTLTLSKAD (SEQ | 3 | | |
| | | ID NO: 252) | | | |
| | | KDSTYSLWSTLTLSK (SEQ ID | 2, 3 | | |
| | | NO: 253) | | | |
| | | KDSTYSLWSTLTLSKA (SEQ ID | 2, 3 | | |
| | | NO: 254) | | | |
| | | KDSTYSLWSTLTLSKAD (SEQ | 3 | | |
| | | ID NO: 255) | | | |
| | | KDSTYSLWSTLTLSKADY (SEQ | 3 | | |
| | | ID NO: 256) | | | |
| | | DSTYSLWSTLTLSK (SEQ ID | 2, 3 | | |
| | | NO: 257) | | | |
| | | DSTYSLWSTLTLSKA (SEQ ID | 2, 3 | | |
| | | NO: 258) | | | |
| | | DSTYSLWSTLTLSKAD (SEQ ID | 3 | | |
| | | NO: 259) | | | |
| | | TYSLWSTLTLSKA (SEQ ID | 3 | | |
| | | NO: 260) | | | |

All in all, the MAPPs and in silico predictions performed on the two RUBY™ bsAbs, with or without optimizing mutations T109P in the CKappa of the H1 chain and S183T in CH1 of the H2 chain, clearly demonstrates that the introduction of these mutations reduced total number of displayed peptides, frequency of donors displaying these peptides as well as the predicted risk score of the displayed peptides. The addition of such optimizing mutations to a RUBY™ bsAb thus holds the potential to reduce the risk of induction of immunogenic responses.

### Example 4—Mutational Strategies to Reduce Levels of Co-Purified Fab Fragments in RUBY™ Bispecific Antibody Purification Processes

### Background & Aim

Although the intricate design of the RUBY™ bispecific antibody (bsAb) format does not allow for formation of any correct sized biproducts, formation of Fab fragments by miss-pairing of chain 2 (L1) and chain 3 (H2) can occur. When such soluble Fab fragments carry a heavy chain variable (VH) domain with origin in the IGHV3 germline gene family they naturally have a protein A binding site located in the VH and this site allows for unwanted co-purification of such Fab fragments. This protein A binding site has been pinpointed to conserved parts of framework regions 1, 3 and the CDRH2 of the VH (Bach, Lewis et al. 2015). To further improve the manufacturability of RUBY™ it would be desirable to engineer molecules were these unwanted Fab products do not have the ability to bind to Protein A, thereby inhibiting its capacity to become co-purified with the correct sized RUBY™ product.

The aim of this study was to evaluate a set (n=3) of mutations (see Table 12) with the potential to disrupt the protein A binding site found in H2 chains carrying VHs with IGHV3 origin. The set of mutations were introduced to 3 different RUBY™ bsAbs with such IGHV3 originating VHs in their H2 chains and evaluated in terms of manufacturability and antigen binding.

### Materials & Methods

Production and Purification of RUBY™ bsAbs

The set of RUBY™ bsAbs listed in Table 12 was expressed together with the 3 parental bsAbs AC_05332, AC_05330 and AC_05302. All RUBY™ bsAbs included in this study also carries a set of format specific mutations, listed in Table 13. The total set of 12 bsAbs were expressed using transient Expi293 HEK (Life Technologies) cells in 30 or 60 mL cultures according to manufacturer's protocol. Purification of produced RUBY bsAbs was performed on the NGC10 (BioRad) system using MabSelect SuRe, 1 mL, columns (GE Healthcare) according to manufacturer's protocol.

TABLE 12
List of parental and mutated RUBY ™ bsAbs
produced and evaluated.

| | H2 chain | | |
|---|---|---|---|
| Parental RUBY ™ | mutation* | New RUBY ID | Specificities |
| AC_05332 | T65E | AC_05370 | 5T4xCD40 |
| AC_05332 | T65A | AC_05371 | 5T4xCD40 |
| AC_05332 | T65I | AC_05372 | 5T4xCD40 |
| AC_05330 | T65E | AC_05373 | CD40x5T4 |
| AC_05330 | T65A | AC_05374 | CD40x5T4 |
| AC_05330 | T65I | AC_05375 | CD40x5T4 |
| AC_05302 | T65E | AC_05376 | CD40x5T4 |
| AC_05302 | T65A | AC_05377 | CD40x5T4 |
| AC_05302 | T65I | AC_05378 | CD40x5T4 |

*According to IGMT numbering

TABLE 13
RUBY ™ specific mutations included in the set of evaluated bsAbs

| RUBY ™ mutations in variable | RUBY ™ mutations in constant |
|---|---|
| domains* | domains** |
| Q44R in the VH and VL of chain H1 | H168A + F170G in CH1 of chain H1 |
| Q44E in the VL of chain L1 | T187E in CH1 of chain H2 |
| Q44E in the VH of chain H2 | S114A + N137K in CKappa of chain H1 |
| | L135Y + S176W in CKappa of chain L1 |

*According to the IMGT numbering system
**According to the EU or Kabat numbering systems

### Evaluation of Manufacturability

Expression yields were calculated using A280 measurements determined by Lunatic (UNchained Labs) after protein A purification. Aggregation and presence of low molecular weight (MW) contaminants, such as soluble Fab fragments, was measured with SE-HPLC in a 1260 Infinity II system (Agilent Technologies) using a TSK gel Super SW mAB HTP 4 μm, 4.6×150 mm column (TOSOH Bioscience) and 100 mM Sodium Phosphate, pH 6.8, 300 mM NaCl as mobile phase at ambient temperature and a flow rate of 0.35 ml/min⁶. Protein quality was assessed using LabChip GXII Touch Capillary Electrophoresis System according to the manufacturer's instructions.

### Evaluation of Antigen Binding Using Dual Target ELISA and Flow Cytometry

Antigen binding was evaluated by dual target ELISA. In brief, relevant antigens were coated at 0.5 μg/ml in PBS in ELISA plates over night at 4° C. After washing in PBS/0.05% Tween 20 (PBST), the plates were blocked with PBST/2% BSA for at least 30 minutes at room temperature before being washed again. Samples serially diluted in PBST/0.5% BSA were then added and allowed to bind for at least 1 hour at room temperature. After washing, plates were incubated with 0.5 μg/mL biotinylated second antigen for at least 1 hour at room temperature. Dual complexed bsAb were detected with HRP-labelled streptavidin. SuperSignal Pico Luminescent was used as substrate and luminescence signals were measured using Fluostar Optima. Binding was compared between parental and mutated RUBY pairs.

Binding to respective targets expressed on cells were evaluated using flow cytometry. I brief, CHO cells modified to express 5T4, B16 cells modified to express CD40 as well as the wildtype CHO and B16 cell lines were incubated with titrated concentrations of RUBY™ bsAbs. Binding of the antibodies was detected using fluorochrome-conjugated anti-human IgG and analyzed using flow cytometry.

### Results & Conclusions

The 12 RUBY™ bsAbs produced at varying levels with yields ranging from 14 to 181 mg/L (Table 14). Except from AC_05375 (T65I) and AC_05376 (T65E), that both produced significantly lower than parental variants, only minor differences could be observed between parental and mutated bsAbs. All mutated RUBY™ bsAbs contained significantly lower amounts of low molecular weight species, such as Fab fragments, as compared to their parental bsAbs after protein A purification. Only small differences in aggregation levels (HMW species) could be observed between parental and mutated bsAbs.

TABLE 14
Summary of manufacturability data of the 12 RUBY ™ bsAbs.

| | | Monomeric | LMWS* | HMWS* |
|---|---|---|---|---|
| BsAb ID | Yield (mg/L) | fraction (%) | (%) | (%) |
| AC_05332 | 173 | 81.8 | 13.1 | 5.1 |
| AC_05370 | 181 | 92.8 | 0.5 | 6.7 |
| AC_05371 | 144 | 92.4 | 0.7 | 7 |
| AC_05372 | 124 | 89.2 | 2.4 | 8.4 |
| AC_05330 | 68 | 85 | 14.5 | 0.5 |
| AC_05373 | 77 | 99.1 | 0 | 0.9 |
| AC_05374 | 69 | 98.9 | 0 | 1.1 |
| AC_05375 | 14 | 95.9 | 0 | 4.1 |
| AC_05302 | 169 | 71.1 | 26.5 | 2.4 |
| AC_05376 | 23 | 94.6 | 0.2 | 5.2 |
| AC_05377 | 106 | 95 | 1.4 | 3.6 |
| AC_05378 | 109 | 95.8 | 0.3 | 3.9 |

*LMWS = Low molecular weight species; HMWS = High molecular weight species

Next, protein quality and levels of co-purified Fab fragments were assesses using LabChip analysis. As seen in Table 15, the introduction of any of the T65E, T65A or T65I mutations dramatically decreased the levels of co-purified Fab fragments after protein a purification. Changes in levels of intact RUBY™ bsAbs (main peak levels, corrected for changes in Fab fragments) induced by the introduction of the protein A site disrupting mutations were in general small, with the exceptions of mutations T65A and T65I to AC_05332 that had dramatic impact on the quality of produced bsAbs.

TABLE 15
LabChip data summarizing levels of intact RUBY ™ bsAbs
and co-purifies soluble Fab fragments.

| | | Fab |
|---|---|---|
| | Main peak | fragments |
| | (~250 kDa, | (~50 kDa, |
| Protein ID | %) | %) |
| AC_05332 | 80.1 | 11.6 |
| AC_05370 | 92.1 | 0 |
| AC_05371 | 62.6 | 0.2 |
| AC_05372 | 11.8 | 0.2 |
| AC_05330 | 86.1 | 14.0 |
| AC_05373 | 92.5 | 0 |
| AC_05374 | 88.3 | 0 |
| AC_05375 | 98.9 | 1.2 |
| AC_05302 | 71.4 | 27.1 |
| AC_05376 | 95.0 | 0 |
| AC_05377 | 98.6 | 1.4 |
| AC_05378 | 97.8 | 0.6 |

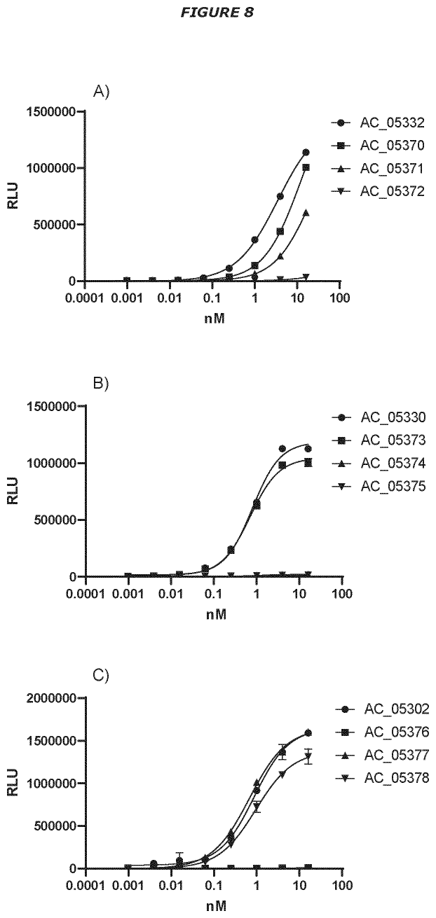

Results from the dual target ELISA analysis are summarized in FIG. 8. The simultaneous binding to both target antigens (5T4 and CD40) of AC_05332 reduced by introduction of all three evaluated mutations, however to much less extent by T65E (AC_05370) and T65A (AC_05371) than T65I (AC_05372). These differences seen between the parental AC_05332 and its mutated variants may also in part be attributed to the reduced protein quality seen for especially the T65A and T65E mutants (see Table 15). Antigen binding capacity of AC_05330 seemed unaffected by the introduction of mutations T65E (AC_05373) while mutations introduced in AC_05374 and AC_05374 abolished the ability of these bsAbs to bind both target antigens simultaneously. AC_05302 did retain antigen binding after introduction of the mutations T65A (AC_05377) and T65I (AC_05378), but not after the introduction of the T65E (AC_05376) mutation.

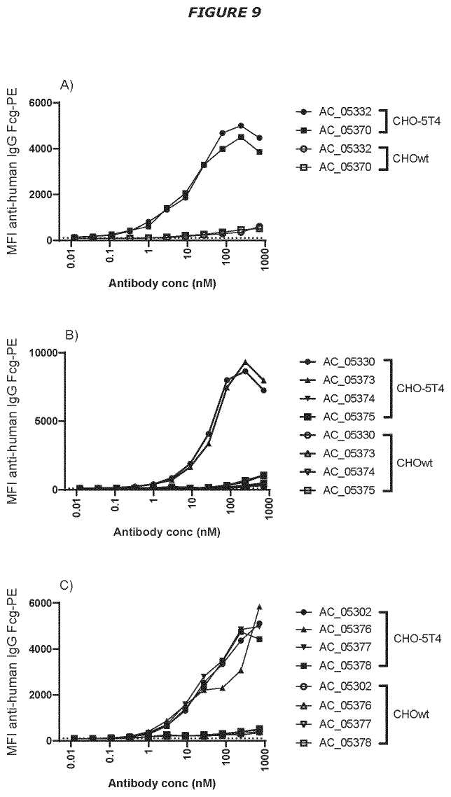

To further assess the impact on target binding by the introduction of the protein A site disrupting mutations binding to cells expressing the two targets were evaluated using flow cytometry (AC_05371 and AC_05372 excluded due to suboptimal protein quality). As can be seen in FIG. 9, binding to 5T4 expressing CHO cells is, as could be expected, not affected by the introduction of mutations in the CD40 binding domains of AC_05332. The binding to 5T4 on cells of AC_05330, AC_05302 and mutant versions thereof correlate with the dual ELISA results described above, with the exception that in this assay all variants of AC_05302 display retained 5T4 binding. AC_05330 mutants on the other hand also in this assay seems to retain antigen binding only with the T65E mutation. Binding to CHOwt cells, not expressing 5T4 is not affected by introduction of any of the studied mutations.

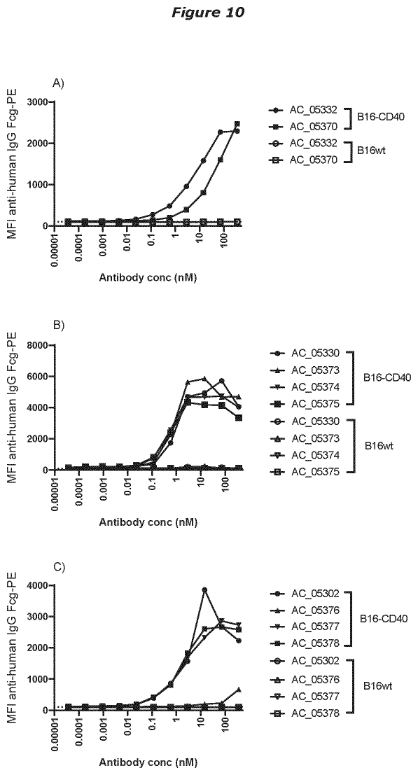

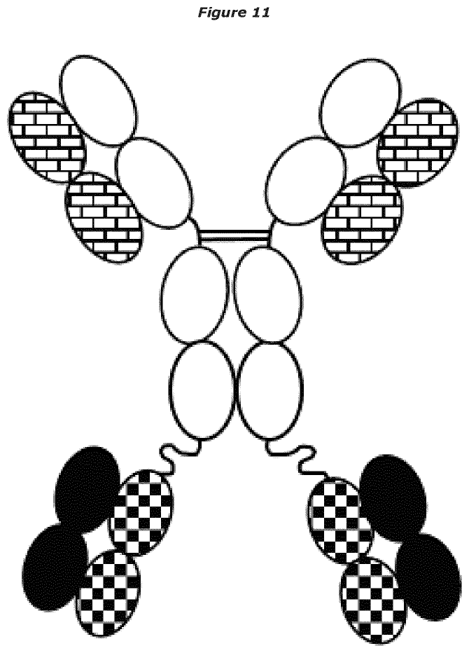

As see in FIG. 10, binding to CD40 expressing B16 cells for AC_05332 and the T65E variant thereof correlate well with ELISA results, with a slight decrease in target binding introduced by the T65E mutation. As expected, all AC_05330 and mutant versions thereof display similar binding to B16 cells, as no mutations in the CD40 binding domains of these RUBY™ bsAbs have been introduced. Apart from an unexpected lack of CD40 binding by AC_05376 (T65E mutant of AC_05302), also AC_05302 and its T65I and T65A mutant versions are unaffected by the introductions of protein A site disrupting mutations in the 5T4 binding domains of these bsAbs. Binding to wtB16 cells, not expressing CD40, is not detected for any of the evaluated RUBY bsAbs.

Importantly, it can be concluded that all three mutations (T65E, T65A and T65I in the VH of the RUBY™ H2 chain) dramatically reduces amounts of copurified Fab fragments in all three bsAbs evaluated in this study, confirming that these mutations indeed do disrupt the protein A binding site in the VH of these molecules. In general, impact on productivity, amount of aggregates and protein quality is limited, with the exception of AC_05332 where the T65A and T65I have a significant impact on protein quality. Impact on target binding, as evaluated using both dual target ELISA and cell binding with FACS, varies between the different mutations introduced to AC_05332, AC_05330 and AC_05302, but for all three bsAbs at least one mutation could be identified that had little or none impact on target binding while significantly reducing the amounts of co-purified Fab fragments.

All in all, these studies demonstrates that the evaluated protein A site disrupting mutations efficiently reduces the amounts of co-purified Fab fragments in the purification step of RUBY bsAbs. The evaluations also demonstrate that it is possible to introduce such mutations without significantly impacting productivity, protein quality or target binding.

### Example 5

A CD40×CEACAM5 bispecific antibody was developed, to test the optimised RUBY™ mutations in assays of therapeutic efficacy, as shown in Table 16.

TABLE 16
Summary of mutations of RUBY ™ ffAC 05337
evaluated in assays for therapeutic efficacy

| | RUBY ™ mutations in constant | Additional optimizing |
|---|---|---|
| RUBY ™ ID | domains | mutations |
| AC_05337 | H168A + F170G in CH1 of chain | — |
| | H1 | |
| ffAC_05337 | T187E in CH1 of chain H2 | S183T in the CH1 of chain H2 |
| | T114A + S137K in CLambda of | |
| | chain H1 | |
| | L135Y + S176W in CKappa of | |
| | chain L1 | |

### Materials and Methods

MB49 CEA overexpressing cells were labeled with the fluorescent dye PKH26 (Sigma-Aldrich) according to manufacturer's instructions. Labeled MB49-CEA cells were heat-shocked at 45° C. for 10 min to induce necrosis, followed by incubation at 37° C. over night. The heat-shocked cells were then centrifuged and the supernatant containing necrotic tumor cell line debris was collected. Raji cells were labeled with the nuclear dye Hoechst 33342 (Thermo Scientific) and cultured with necrotic debris and titrated antibodies (ffAC_05337 or 1132). Images were captured using a Cytation 5 live cell imager (BioTek) and the number of PKH26-stained tumor debris co-localized with Hoechst-stained Raji cells was quantified using Gen5 software (BioTek).

### Results

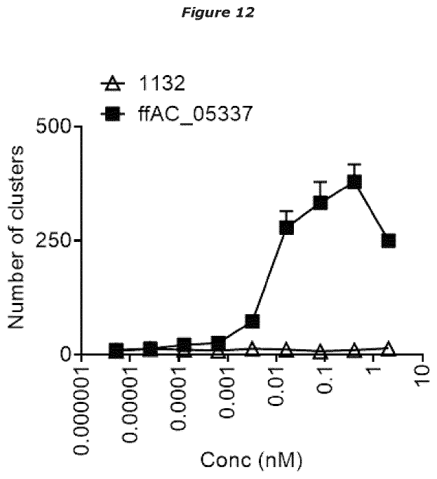

A dose-dependent increase in clusters of necrotic debris from a CEA-transfected MB49 tumor cell line with Raji cells was seen when the CD40×CEA targeting ffAC_05337 bsAb was added, but not with the monospecific CD40 Ab 1132 (as shown in FIG. 12).

### Example 6—Functional Assays Using Cells Obtained from Primary Human Colorectal Cancer Patients

Dissociated primary cells from colorectal cancer patients were purchased from Discovery Life Sciences (Huntsville, AL). Directly after thawing, DTCs were counted using NucleoCounter® NC-200™ (Chemometec, Denmark) and 20,000 viable cells were pipetted into each well. The cancer cells were used to assess functionality in the CD40 bioassay, or alternatively the ability of the primary cancer cells to activate the immune cells in the same tumor sample was analyzed. 200,000 viable cells were pipetted into a Nunc UpCell 96-well plate (Thermo Scientific, 174897). Next, ATOR-4066 or controls were added into the wells. The plate was incubated for 48 hours in a 37° C., 5% CO2 incubator. Next, the cells were harvested, and analyzed by flow cytometry.

TABLE 17
FACs panel for activation staining (22 tubes were stained)

| | | | |
|---|---|---|---|
| CD40 | APC | 555591 | BD Pharmingen |
| CD86 | PE | 555658 | BD Pharmingen |
| CD45 | V450 | 560367 | BD Horizon |
| CD14 | PerCP-Cy5.5 | 562692 | BD Pharmingen |
| CD83 | PE-Cy7 | 561132 | BD Pharmingen |
| CD3 | FITC | 555332 | BD Pharmingen |
| CD56 | FITC | 562794 | BD Pharmingen |

### Results

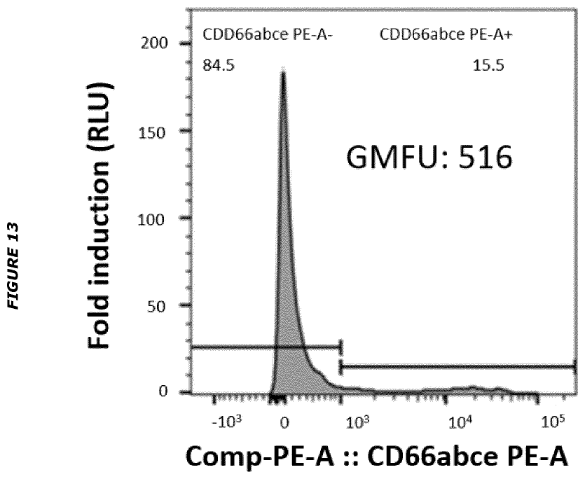

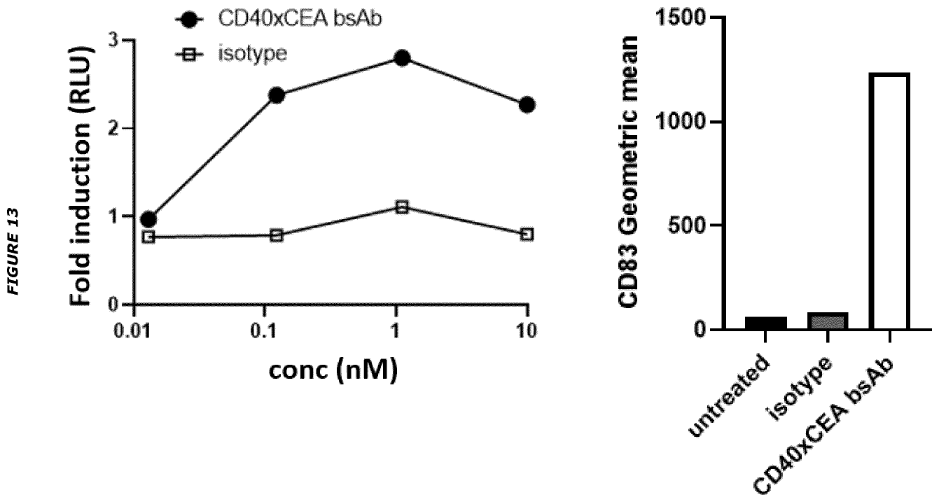

First, it was demonstrated that the CEA densities in patient derived tumors were sufficient to provide cross-linking and induce CD40 stimulation using a reporter cell assay. The results demonstrated patient derived cancer cells can induce similar cross-linking and CD40 activation as the cell lines (FIG. 13). Secondly, when culturing dissociated cells from patient derived colon tumors, it was demonstrated that a CD40×CEA bsAb (ffAC_05337) could activate tumor infiltrating immune cells (FIG. 13).

### Example 7

### Materials and Methods

### Human Samples

The collection of the tonsillar cancer sample at Lund University Hospital was approved by the Swedish Ethical Review Authority (ref. no. 2017/580), and the participating patient granted written informed consent.

### Cell Isolation and Coculture

The tonsillar cancer biopsy was cut into small fragments in RPMI 1640 medium (ThermoFisher Scientific) supplemented with 0.1 mg/mL gentamycin (Sigma-Aldrich). The tissue fragments were enzymatically digested with Collagenase IV (Sigma-Aldrich) (2.0 mg/mL) and DNase I (Sigma Aldrich) (200 Kunits/mL) for 20 minutes at 37° C. Cells were filtered using a 70 μm cell strainer (BD Biosciences) and stained with CD3− PerCPCy5.5, VS620-PECF594, CD45-APCH7 and HLA-DR-BV711 for cell sorting using FACSAria IIu (BD Biosciences). 104 viable CD45+ HLA-DR+ CD3− cells were sorted directly into 96-well flat-bottom plates (Nunc UpCell, ThermoFisher Scientific) pre-seeded with 6×104 UV-irradiated CHO-CEA cells, per well. 19 nM of CD40×CEA bsAbs, CD40 mAbs or isotype controls were added to the cocultures for 13 h, after which the supernatants were collected, and the cells were washed and blocked for non-specific binding with ChromPure mouse IgG (Jackson ImmunoResearch) for 15 min at room temperature. Cells were immediately stained with fluorochrome-coupled antibodies (Supplementary Table 1) for flow cytometric analysis using a FACSAria IIu instrument (BD Biosciences). Cytokine analysis was performed using Bio-Plex Pro Human Cytokine 17-plex Assay on the Bio-PlexR 200 system (Bio-Rad Lab).

TABLE 18
Antibodies used for flow cytometry.

| | | | |
|---|---|---|---|
| Antigen | Fluorophore | Clone | Company |

Human cells

| | | | |
|---|---|---|---|
| CD45 | APCH7 | 2D1 | BD Biosciences |
| HLA-DR | BV711 | G46-6 | BD Biosciences |
| CD11c | BV510 | B-ly6 | BD Biosciences |
| XCR1 | PE | S15046E | BioLegend |
| CD1C | BV786 | L161 | BioLegend |
| CD14 | FITC | TUK4 | Invitrogen |
| CCR2 | APC | K036C2 | BioLegend |
| CD123 | AF700 | 6H6 | BioLegend |
| CD19 | PerCP Cy5.5 | HIB19 | BD Biosciences |
| CD20 | PerCP Cy5.5 | 2H7 | BD Biosciences |
| CD86 | BV605 | IT2.2 | BioLegend |
| CD40 | PE-Cy7 | 5C3 | BD Biosciences |
| CCR7 | BV421 | 2-L1-A | BD Biosciences |

### Results

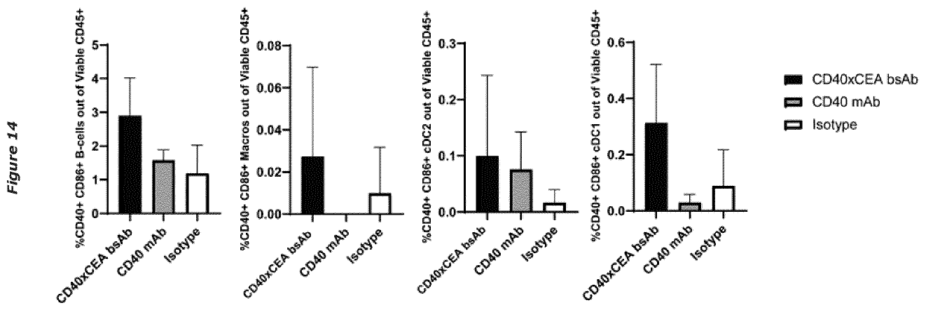

As shown in FIG. 14, simultaneous binding of CD40 and CEA by CD40×CEA bsAbs (ffAC_05337) mediates activation of tonsillar cancer APCs in vitro.

### Example 8: Anti-Tumor Effect of CD40-CEACAM5 Bispecific Antibody ffAC_05337

### Background and Aim

ffAC_05337 is a CD40-CEA bispecific antibody in the RUBY™ format. The antibody has been LALA-mutated to silence Fcg receptor binding.

The aim of this study was to evaluate the anti-tumor effect of ffAC_05337 and a CD40 mAb in human CD40 transgenic (hCD40tg) mice inoculated with a CEACAM5-transfected murine tumor cell line called MC38-CEACAM5 2.

### Materials and Methods

Female hCD40tg mice of 9 weeks of age were inoculated with 1×10⁶ MC38-CEACAM5 2 cells (obtained from Kerafast) s.c. in the right flank. On days 7, 10, and 13 after inoculation, the mice were administered i.p. with 100 μg of wildtype CD40 monospecific antibody, 1132, or 167 μg of the CD40-CEACAM5 bsAb ffAC_05337. A group of vehicle-treated mice was also included. The tumors were frequently measured with a caliper in width (w), length (l) and height (h) and the tumor volume was calculated using the formula: (w/2×l/2×h/2×n×(4/3)).

### Results and Conclusions

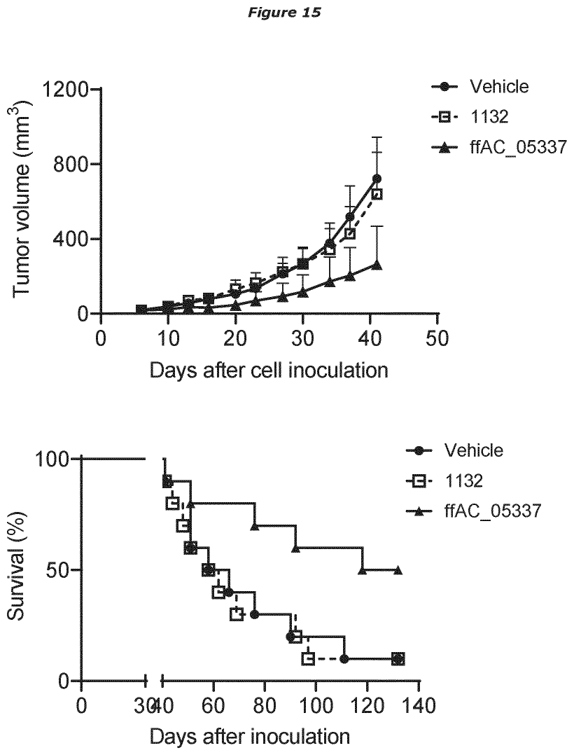

The data demonstrated that treatment with the CD40-CEACAM5 bsAb ffAC_05337 but not the CD40 mAb 1132 reduced the MC38-CEACAM5 tumor volume compared to vehicle-treated mice (FIG. 15). Further, treatment with ffAC_05337 but not 1132 led to improved survival compared to vehicle-treated mice (FIG. 15).

### Example 9—Functional Assays Using Cells Obtained from Primary Human Gastric Cancer Patients

Dissociated primary cells from gastric cancer patients were purchased from Discovery Life Sciences (Huntsville, AL). Directly after thawing, DTCs were counted using NucleoCounter® NC-200™ (Chemometec, Denmark) and 20,000 viable cells were pipetted into each well. The cancer cells were used to assess functionality in the CD40 bioassay, or alternatively the ability of the primary cancer cells to activate the immune cells in the same tumor sample was analyzed. 200,000 viable cells were pipetted into a Nunc UpCell 96-well plate (Thermo Scientific, 174897). Next, CD40×CEA bsAb (ffAC_05337) or controls were added into the wells. The plate was incubated for 48 hours in a 37° C., 5% CO2 incubator. Next, the cells were harvested, and analyzed by flow cytometry.

TABLE 19
FACs panel for activation staining

| | | | |
|---|---|---|---|
| CD40 | APC | 555591 | BD Pharmingen |
| CD86 | PE | 555658 | BD Pharmingen |
| CD45 | V450 | 560367 | BD Horizon |
| CD14 | PerCP-Cy5.5 | 562692 | BD Pharmingen |
| CD83 | PE-Cy7 | 561132 | BD Pharmingen |
| CD3 | FITC | 555332 | BD Pharmingen |
| CD56 | FITC | 562794 | BD Pharmingen |

### Results

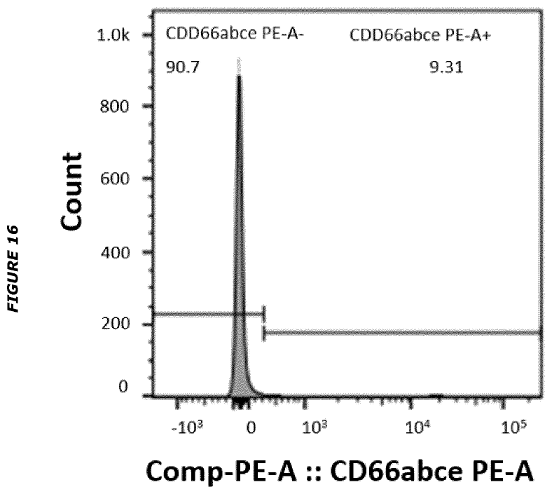

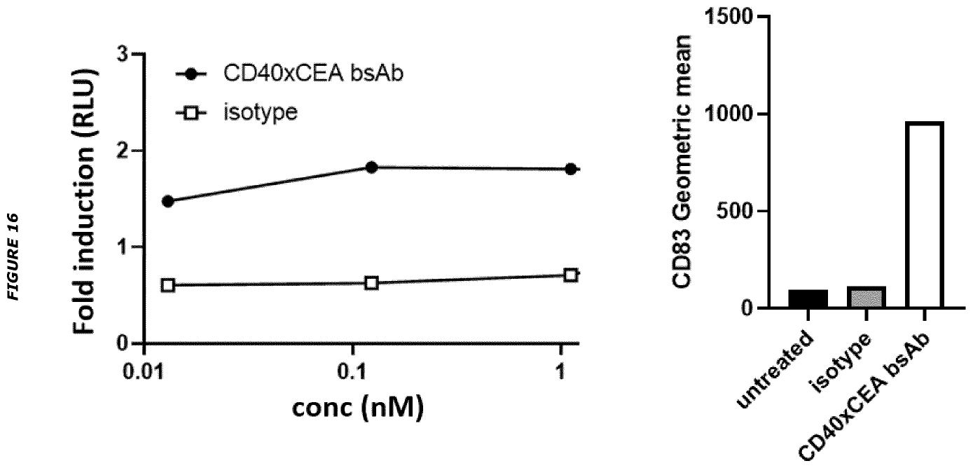

First, it was demonstrated that the CEA densities in patient derived tumors were sufficient to provide cross-linking and induce CD40 stimulation using a reporter cell assay. The results demonstrated that patient derived cancer cells can induce similar cross-linking and CD40 activation as the cell lines (FIG. 32). Secondly, when culturing dissociated cells from patient derived gastric tumors, it was demonstrated that a CD40×CEA bsAb (ffAC_05337) could activate tumor infiltrating immune cells (FIG. 16).

### Example 10—Evaluation of the CD40 Agonistic Function in the Presence of Soluble CEA Using the CD40 Reporter Assay

### Aim and Background

The aim of this study was to assess the CD40 agonistic function of the bispecific antibody ffAC_05337 using the CD40 reporter assay in the presence of CEA expressing cells and physiological relevant soluble CEA concentrations. CD40 crosslinking will be mediated by simultaneous binding of CD40, expressed on CD40 reporter cells, and CEA expressed on CHO cells or CEA expressing human tumor cells. In addition, since high levels of soluble CEA can be detected systemically in cancer patients, the agonistic function was assessed in the presence of physiological relevant concentrations of soluble CEA.

### Materials and Methods

Agonistic function of the ffAC_05337 was evaluated using a CD40 reporter assay (Promega, CD40 Bioassay Kit CS JA2155). The assay was performed according to the manufacturer's protocol. In brief, CD40 reporter cells and titrating concentrations of ffAC_05337 were diluted in RPMI containing 10% FCS and added to the assay plates before the addition of CEA transfected CHO, CHO wt or CEA expressing human tumor cells. In addition, the assay was performed in the presence of 0.5, 1, 5, 25 or 50 μg/ml soluble CEA. The assay plates were incubated for 6 h at 37° C. until addition of Bio-Glo™ Luciferase Assay Detection solution and analyzed in the BMG ELISA plate reader.

### RESULTS AND CONCLUSIONS

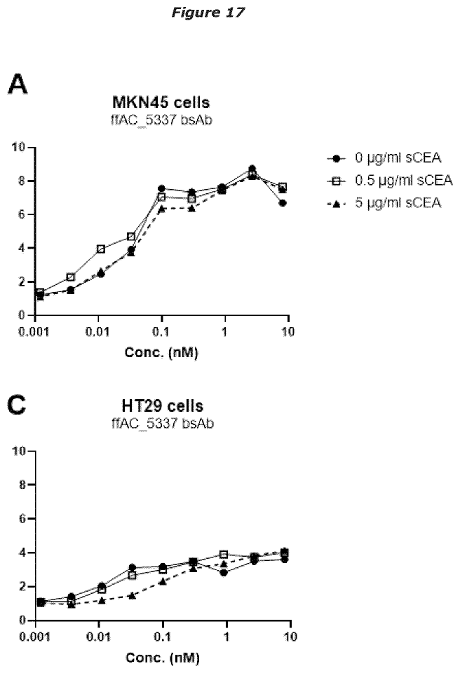

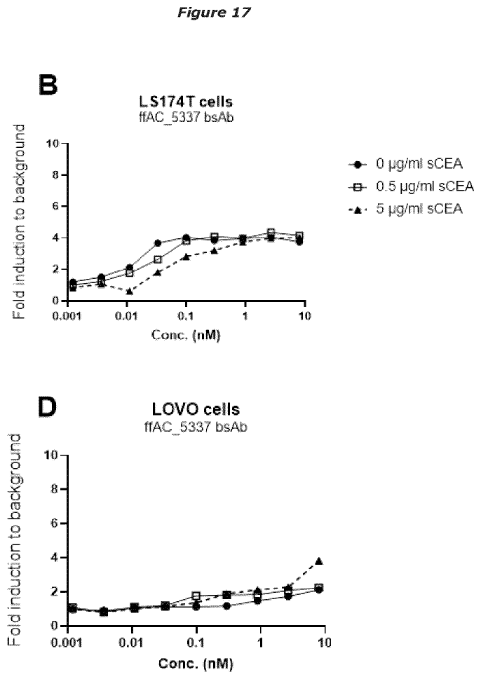

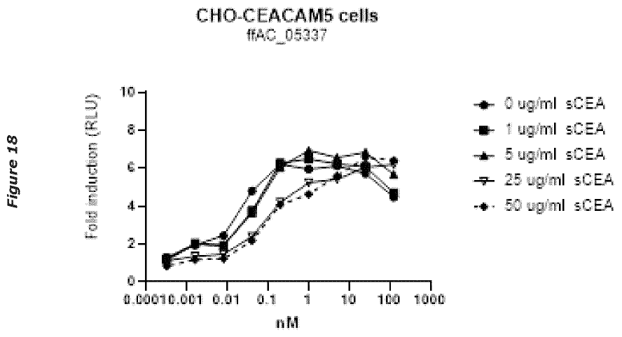

The results show that the bispecific antibody ffAC_05337 induce CD40 activation in the presence of CEA and the potency and efficacy is unaffected by the presence of soluble CEA in the cultures (FIGS. 17 and 18). A minor decrease in the efficacy of ffAC_05337 can be observed in the presence of 25 and 50 μg/ml soluble CEA (FIG. 18).

Additional sequences relevant to the invention
AC_05293
Chain H1
(SEQ ID NO: 261)
EVQLLESGGGLVQPGGSLRLSCAASGFTFSSYAMSWVRRAPGKGLEWVSGIGSYGGGTYYAD
SVKGRFTISRDNSKNTLYLQMNSLRAEDTAVYYCARYVNFGMDYWGQGTLVTVSSASTKGPSV
FPLAPSSKSTSGGTAALGCLVKDYFPEPVTVSWNSGALTSGVATGPAVLQSSGLYSLSSVVTVP
SSSLGTQTYICNVNHKPSNTKVDKKVEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLS
PGKGGGGSGGGGSGGGGSELVMTQSPSSLTVTAGEKVTMSCKSSQSLLNSGNQKNYLTWYQ
RKPGQPPKLLIYWASTRESGVPDRFTGSGSGTDFTLTISSVQAEDLAVYYCQNDYSYPLTFGAGT
KLEIKRTVAAPAVFIFPPSDEQLKSGTASVVCLLKNFYPREAKVQWKVDNALQSGNSQESVTEQ
DSKDSTYSLSSTLTLSKADYEKHKVYACEVTHQGLSSPVTKSFNRGEC
Chain H2
(SEQ ID NO: 262)
EVQLLEQSGAELVRPGTSVKISCKASGYAFTNYWLGWVKERPGHGLEWIGDIFPGSGNIHYNE
KFKGKATLTADKSSSTAYMQLSSLTFEDSAVYFCARLRNWDEPMDYWGQGTTVTVSSASTKGP
SVFPLAPSSKSTSGGTAALGCLVKDYFPEPVTVSWNSGALTSGVHTFPAVLQSSGLYSLSSVVE
VPSSSLGTQTYICNVNHKPSNTKVDKKVEPKSC
Chain L1
(SEQ ID NO: 263)
DIQMTQSPSSLSASVGDRVTITCRASQSISSYLNWYQEKPGKAPKLLIYAASSLQSGVPSRFSG
SGSGTDFTLTISSLQPEDFATYYCQQYGRNPPTFGQGTKLEIKRTVAAPSVFIFPPSDEQLKSGTA
SVVCYLNNFYPREAKVQWKVDNALQSGNSQESVTEQDSKDSTYSLWSTLTLSKADYEKHKVYA
CEVTHQGLSSPVTKSFNRGEC
Multi46
Chain H1
(SEQ ID NO: 264)
EVQLLESGGGLVQPGGSLRLSCAASGFTFSSYAMSWVRRAPGKGLEWVSGIGSYGGGTYYAD
SVKGRFTISRDNSKNTLYLQMNSLRAEDTAVYYCARYVNFGMDYWGQGTLVTVSSASTKGPSV
FPLAPSSKSTSGGTAALGCLVKDYFPEPVTVSWNSGALTSGVATGPAVLQSSGLYSLSSVVTVP
SSSLGTQTYICNVNHKPSNTKVDKKVEPKSCDKTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLS
PGKGGGGSGGGGSGGGGSDIQMTQSPSSLSASVGDRVTITCRASQSISSYLNWYQRKPGKAP
KLLIYAASSLQSGVPSRFSGSGSGTDFTLTISSLQPEDFATYYCQQSSHGPLLTFGQGTKLEIKRP
VAAPAVFIFPPSDEQLKSGTASVVCLLKNFYPREAKVQWKVDNALQSGNSQESVTEQDSKDST
YSLSSTLTLSKADYEKHKVYACEVTHQGLSSPVTKSFNRGEC
Chain H2
(SEQ ID NO: 265)
QVQLVQSGAEVKKPGSSVKVSCKASGGTFGYYAIHWVREAPGQGLEWMGGIGSIFGTANYAQ
KFQGRVTITADESTSTAYMELSSLRSEDTAVYYCARAWSSDHMDYWGQGTLVTVSSASTKGPS
VFPLAPSSKSTSGGTAALGCLVKDYFPEPVTVSWNSGALTSGVHTFPAVLQSSGLYSLTSVVEVP
SSSLGTQTYICNVNHKPSNTKVDKKVEPKSC
Chain L1
(SEQ ID NO: 266)
DIQMTQSPSSLSASVGDRVTITCRASQSISSYLNWYQEKPGKAPKLLIYAASSLQSGVPSRFSG
SGSGTDFTLTISSLQPEDFATYYCQQYGRNPPTFGQGTKLEIKRTVAAPSVFIFPPSDEQLKSGTA
SVVCYLNNFYPREAKVQWKVDNALQSGNSQESVTEQDSKDSTYSLWSTLTLSKADYEKHKVYA
CEVTHQGLSSPVTKSFNRGEC
AC_05332
Chain H1
(SEQ ID NO: 267)
EVQLLESGGGLVQPGGSLRLSCAASGFTFSSYAMSWVRRAPGKGLEWVSAISGSGGSTYY
ADSVKGRFTISRDNSKNTLYLQMNSLRAEDTAVYYCARYYGGYYSAWMDYWGQGTLVTVS
SASTKGPSVFPLAPSSKSTSGGTAALGCLVKDYFPEPVTVSWNSGALTSGVATGPAVLQS
SGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGKGGGGSGGGGSGGGGSDIQMTQSPSSLSAS
VGDRVTITCRASQSISSYLNWYQRKPGKAPKLLIYAASSLQSGVPSRFSGSGSGTDFTLT
ISSLQPEDFATYYCQQYGRNPPTFGQGTKLEIKRTVAAPAVFIFPPSDEQLKSGTASVVC
LLKNFYPREAKVQWKVDNALQSGNSQESVTEQDSKDSTYSLSSTLTLSKADYEKHKVYAC
EVTHQGLSSPVTKSFNRGEC
Chain L1
(SEQ ID NO: 268)
DIQMTQSPSSLSASVGDRVTITCRASQSISSYLNWYQEKPGKAPKLLIYAASSLQSGVPS
RFSGSGSGTDFTLTISSLQPEDFATYYCQQTYGYLHTFGQGTKLEIKRTVAAPSVFIFPP
SDEQLKSGTASVVCYLNNFYPREAKVQWKVDNALQSGNSQESVTEQDSKDSTYSLWSTLT
LSKADYEKHKVYACEVTHQGLSSPVTKSFNRGEC
Chain H2
(SEQ ID NO: 269)
EVQLLESGGGLVQPGGSLRLSCAASGFTFSSYAMSWVREAPGKGLEWVSGIGSYGGGTYY
ADSVKGRFTISRDNSKNTLYLQMNSLRAEDTAVYYCARYVNFGMDYWGQGTLVTVSSAST
KGPSVFPLAPSSKSTSGGTAALGCLVKDYFPEPVTVSWNSGALTSGVHTFPAVLQSSGLY
SLSSVVEVPSSSLGTQTYICNVNHKPSNTKVDKKVEPKSC
AC_05330
Chain H1
(SEQ ID NO: 270)
EVQLLESGGGLVQPGGSLRLSCAASGFTFSTYGMHWVRRAPGKGLEWLSYISGGSSYIFY
ADSVRGRFTISRDNSENALYLQMNSLRAEDTAVYYCARILRGGSGMDLWGQGTLVTVSSA
STKGPSVFPLAPSSKSTSGGTAALGCLVKDYFPEPVTVSWNSGALTSGVATGPAVLQSSG
LYSLSSVVTVPSSSLGTQTYICNVNHKPSNTKVDKKVEPKSCDKTHTCPPCPAPEAAGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQ
QGNVFSCSVMHEALHNHYTQKSLSLSPGKGGGGSGGGGSGGGGSDIQMTQSPSSLSASVG
DRVTITCRASQSISSYLNWYQRKPGKAPKLLIYAASSLQSGVPSRFSGSGSGTDFTLTIS
SLQPEDFATYYCQQTYGYLHTFGQGTKLEIKRTVAAPAVFIFPPSDEQLKSGTASVVCLL
KNFYPREAKVQWKVDNALQSGNSQESVTEQDSKDSTYSLSSTLTLSKADYEKHKVYACEV
THQGLSSPVTKSFNRGEC
Chain L1
(SEQ ID NO: 271)
QSVLTQPPSASGTPGQRVTISCTGSSSNIGAGYNVYWYQELPGTAPKLLIYGNINRPSGV
PDRFSGSKSGTSASLAISGLRSEDEADYYCAAWDKSISGLVFGGGTKLTVLGQPKAAPSV
TLFPPSSEELQANKATLVCYISDFYPGAVTVAWKADSSPVKAGVETTTPSKQSNNKYAAW
SYLSLTPEQWKSHRSYSCQVTHEGSTVEKTVAPTECS
Chain H2
(SEQ ID NO: 272)
EVQLLESGGGLVQPGGSLRLSCAASGFTFSSYAMSWVREAPGKGLEWVSAISGSGGSTYY
ADSVKGRFTISRDNSKNTLYLQMNSLRAEDTAVYYCARYYGGYYSAWMDYWGQGTLVTVS
SASTKGPSVFPLAPSSKSTSGGTAALGCLVKDYFPEPVTVSWNSGALTSGVHTFPAVLQS
SGLYSLSSVVEVPSSSLGTQTYICNVNHKPSNTKVDKKVEPKSC
AC_05302
Chain H1
(SEQ ID NO: 273)
EVQLLESGGGLVQPGGSLRLSCAASGFTFSSYAMSWVRRAPGKGLEWVSGIGSYGGGTYY
ADSVKGRFTISRDNSKNTLYLQMNSLRAEDTAVYYCARYVNFGMDYWGQGTLVTVSSAST
KGPSVFPLAPSSKSTSGGTAALGCLVKDYFPEPVTVSWNSGALTSGVATGPAVLQSSGLY
SLSSVVTVPSSSLGTQTYICNVNHKPSNTKVDKKVEPKSCDKTHTCPPCPAPEAAGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQG
NVFSCSVMHEALHNHYTQKSLSLSPGKGGGGGGGGSGGGGSDIQMTQSPSSLSASVGDR
VTITCRASQSISSYLNWYQRKPGKAPKLLIYAASSLQSGVPSRFSGSGSGTDFTLTISSL
QPEDFATYYCQQTYGYLHTFGQGTKLEIKRTVAAPAVFIFPPSDEQLKSGTASVVCLLKN
FYPREAKVQWKVDNALQSGNSQESVTEQDSKDSTYSLSSTLTLSKADYEKHKVYACEVTH
QGLSSPVTKSFNRGEC
Chain L1
(SEQ ID NO: 274)
DIQMTQSPSSLSASVGDRVTITCRASQSISSYLNWYQEKPGKAPKLLIYAASSLQSGVPS
RFSGSGSGTDFTLTISSLQPEDFATYYCQQYGRNPPTFGQGTKLEIKRTVAAPSVFIFPP
SDEQLKSGTASVVCYLNNFYPREAKVQWKVDNALQSGNSQESVTEQDSKDSTYSLWSTLT
LSKADYEKHKVYACEVTHQGLSSPVTKSFNRGEC
Chain H2
(SEQ ID NO: 275)
EVQLLESGGGLVQPGGSLRLSCAASGFTFSSYAMSWVREAPGKGLEWVSAISGSGGSTYY
ADSVKGRFTISRDNSKNTLYLQMNSLRAEDTAVYYCARYYGGYYSAWMDYWGQGTLVTVS
SASTKGPSVFPLAPSSKSTSGGTAALGCLVKDYFPEPVTVSWNSGALTSGVHTFPAVLQS
SGLYSLSSVVEVPSSSLGTQTYICNVNHKPSNTKVDKKVEPKSC
ffAC_05337
Chain H1
(SEQ ID NO: 276)
EVQLLESGGGLVQPGGSLRLSCAASGFTFSSSYMGWVRRAPGKGLEWVSSIGSGSYSTSY
ADSVKGRFTISRDNSKNTLYLQMNSLRAEDTAVYYCARYPSVPFPLHLDYWGQGTLVTVS
SASTKGPSVFPLAPSSKSTSGGTAALGCLVKDYFPEPVTVSWNSGALTSGVATGPAVLQS
SGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGKGGGGSGGGGSGGGGSQSVLTQPPSASGTP
GQRVTISCTGSSSNIGAGYNVYWYQRLPGTAPKLLIYGNINRPSGVPDRFSGSKSGTSAS
LAISGLRSEDEADYYCAAWDKSISGLVFGGGTKLTVLGQPKAAPAVTLFPPSSEELQANK
ATLVCLIKDFYPGAVTVAWKADSSPVKAGVETTTPSKQSNNKYAAWSYLSLTPEQWKSHR
SYSCQVTHEGSTVEKTVAPTECS
Chain L1
(SEQ ID NO: 277)
DIQMTQSPSSLSASVGDRVTITCRASQSISSYLNWYQEKPGKAPKLLIYAASSLQSGVPS
RFSGSGSGTDFTLTISSLQPEDFATYYCQQAGNPHTFGQGTKLEIKRTVAAPSVFIFPPS
DEQLKSGTASVVCYLNNFYPREAKVQWKVDNALQSGNSQESVTEQDSKDSTYSLWSTLTL
SKADYEKHKVYACEVTHQGLSSPVTKSFNRGEC
Chain H2
(SEQ ID NO: 278)
EVQLLESGGGLVQPGGSLRLSCAASGFTFSTYGMHWVREAPGKGLEWLSYISGGSSYIFY
ADSVKGRFTISRDNSKNTLYLQMNSLRAEDTAVYYCARILRGGSGMDLWGQGTLVTVSSA
STKGPSVFPLAPSSKSTSGGTAALGCLVKDYFPEPVTVSWNSGALTSGVHTFPAVLQSSG
LYSLTSVVEVPSSSLGTQTYICNVNHKPSNTKVDKKVEPKSC
AC_05337 (parental of ffAC_05337)
Chain H1
(SEQ ID NO: 279)
EVQLLESGGGLVQPGGSLRLSCAASGFTFSSSYMGWVRRAPGKGLEWVSSIGSGSYSTSY
ADSVKGRFTISRDNSKNTLYLQMNSLRAEDTAVYYCARYPSVPFPLHLDYWGQGTLVTVS
SASTKGPSVFPLAPSSKSTSGGTAALGCLVKDYFPEPVTVSWNSGALTSGVATGPAVLQS
SGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSR
WQQGNVFSCSVMHEALHNHYTQKSLSLSPGKGGGGSGGGGSGGGGSQSVLTQPPSASGTP
GQRVTISCTGSSSNIGAGYNVYWYQRLPGTAPKLLIYGNINRPSGVPDRFSGSKSGTSAS
LAISGLRSEDEADYYCAAWDKSISGLVFGGGTKLTVLGQPKAAPAVTLFPPSSEELQANK
ATLVCLIKDFYPGAVTVAWKADSSPVKAGVETTTPSKQSNNKYAAWSYLSLTPEQWKSHR
SYSCQVTHEGSTVEKTVAPTECS
Chain L1
(SEQ ID NO: 280)
DIQMTQSPSSLSASVGDRVTITCRASQSISSYLNWYQEKPGKAPKLLIYAASSLQSGVPS
RFSGSGSGTDFTLTISSLQPEDFATYYCQQAGNPHTFGQGTKLEIKRTVAAPSVFIFPPS
DEQLKSGTASVVCYLNNFYPREAKVQWKVDNALQSGNSQESVTEQDSKDSTYSLWSTLTL
SKADYEKHKVYACEVTHQGLSSPVTKSFNRGEC
Chain H2
(SEQ ID NO: 281)
EVQLLESGGGLVQPGGSLRLSCAASGFTFSTYGMHWVREAPGKGLEWLSYISGGSSYIFY
ADSVRGRFTISRDNSENALYLQMNSLRAEDTAVYYCARILRGGSGMDLWGQGTLVTVSSA
STKGPSVFPLAPSSKSTSGGTAALGCLVKDYFPEPVTVSWNSGALTSGVHTFPAVLQSSG
LYSLSSVVEVPSSSLGTQTYICNVNHKPSNTKVDKKVEPKSC
1132
Heavy chain
(SEQ ID NO: 282)
EVQLLESGGGLVQPGGSLRLSCAASGFTFSSYAMSWVRQAPGKGLEWVSGIGSYGGGTYYAD
SVKGRFTISRDNSKNTLYLQMNSLRAEDTAVYYCARYVNFGMDYWGQGTLVTVSSASTKGPSV
FPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPG
K
Light chain
(SEQ ID NO: 283)
DIQMTQSPSSLSASVGDRVTITCRASQSISSYLNWYQQKPGKAPKLLIYAASSLQSGVPSRFSG
SGSGTDFTLTISSLQPEDFATYYCQQYGRNPPTFGQGTKLEIKRTVAAPSVFIFPPSDEQLKSGTA
SVVCLLNNFYPREAKVQWKVDNALQSGNSQESVTEQDSKDSTYSLSSTLTLSKADYEKHKVYA
CEVTHQGLSSPVTKSFNRGEC

1: A bispecific antibody comprising:
(a) an immunoglobulin molecule having specificity for a first antigen, the immunoglobulin molecule comprising a first heavy chain region and a first light chain region; and
(b) at least one appended Fab fragment having specificity for a second antigen, the appended Fab fragment comprising a second heavy chain region and a second light chain region;
wherein the second light chain region is fused to the C-terminus of the first heavy chain region;
wherein the immunoglobulin molecule and the appended Fab fragment comprise an antibody VL region, an antibody VH region, a CH1 region and a CKappa or CLambda region, and
i. wherein the bispecific antibody comprises one or more primary mutations to promote an association of the first heavy chain region with the first light chain region and the second heavy chain region and the second light chain region, and wherein such mutations increase the predicted risk of inducing immunogenic responses and/or increase the immunogenicity of the bispecific antibody; and
ii. wherein the bispecific antibody comprises one or more secondary mutations to reduce or avoid the predicted risk of inducing immunogenic responses and/or immunogenicity caused by the one or more primary mutations.

2: The bispecific antibody according to claim 1, wherein the increase in the immunogenicity is characterised as a higher predicted immunogenicity risk when compared to the predicted immunogenicity risk of a primary mutation control polypeptide and/or a higher immunogenicity when compared to the immunogenicity of a primary mutation control polypeptide, preferably the primary mutation control polypeptide does not comprise the one or more primary mutations, optionally wherein the primary mutation control polypeptide comprises an amino acid sequence that is the same as the polypeptide prior to the one or more primary mutations being introduced.

3. (canceled)

4: The bispecific antibody according to claim 1, wherein the reduction in the immunogenicity is characterised as a lower predicted immunogenicity risk when compared to the predicted immunogenicity risk of a first secondary mutation control polypeptide and/or a lower immunogenicity when compared to the immunogenicity of a first secondary mutation control polypeptide, preferably the first secondary mutation control polypeptide comprises the one or more primary mutations but does not comprise the one or more secondary mutations.

5: The bispecific antibody according to claim 4, wherein the first secondary mutation control polypeptide comprises an amino acid sequence that is the same as the polypeptide comprising one or more of the primary mutations prior to the one or more secondary mutations being introduced.

6: The bispecific antibody according to claim 1, wherein the avoidance of immunogenicity is characterised as a similar, or the same, predicted immunogenicity risk when compared to the predicted immunogenicity risk of a second secondary mutation control polypeptide and/or immunogenicity when compared to the immunogenicity of a second secondary mutation control polypeptide, preferably the second secondary mutation control polypeptide does not comprise of the one or more primary mutations or the one or more secondary mutations.

7: The bispecific antibody according to claim 6, wherein the second secondary mutation control polypeptide comprises an amino acid sequence that is the same as the polypeptide prior to the one or more primary mutations being introduced and prior to the one or more secondary mutations being introduced.

8: The bispecific antibody according to claim 1, wherein the immunogenicity is antigen-presenting cell (APC) dependent immunogenicity and/or T-cell dependent immunogenicity, optionally wherein the APC is a dendritic cell.

9: The bispecific antibody according to claim 1, wherein the secondary mutations reduce or avoid the bispecific antibody binding to an MHC class II and/or a T-cell receptor (TCR).

10-16. (canceled)

17: The bispecific antibody according to claim 1, wherein the heavy chain region comprises a CH1 domain, wherein the secondary mutation is at position 180, 183, and/or 188 in the CH1 domain.

18: The bispecific antibody according to claim 1, wherein the light chain region comprises a CKappa or Clambda (preferably CKappa) domain, wherein the secondary mutation is at position 111 and/or 109 in the CKappa or Clambda (preferably CKappa) domain.

19. (canceled)

20: The bispecific antibody according to claim 1, wherein the secondary mutations are at one or more (preferably two or more) of the following positions: 180 in the CH1 domain; 183 in the CH1 domain; 188 in the CH1 domain; 111 in the CKappa or CLambda (preferably CKappa) domain; 109 in the CKappa or CLambda (preferably CKappa) domain; and 126 in the VL domain.

21-23. (canceled)

24: The bispecific antibody according to claim 1, wherein the secondary mutation is: S183T and/or T109P.

25-39. (canceled)

40: The bispecific antibody according to according to claim 1, wherein the immunoglobulin molecule comprises two copies of a first heavy chain polypeptide and/or two copies of a first light chain polypeptide.

41: The bispecific antibody according to according to claim 1, wherein the antibody comprises two Fab fragments according to (b).

42: The bispecific antibody according to claim 1, wherein the immunoglobulin molecule comprises two copies of a first heavy chain polypeptide and two copies of a first light chain polypeptide,
and the bispecific antibody further comprises two Fab fragments according to (b),
and the first Fab fragment is fused to the C-terminus of the first copy of the first heavy chain polypeptide via the light chain polypeptide of the Fab fragment;
and the second Fab fragment is fused to the C-terminus of the second copy of the first heavy chain polypeptide via the light chain polypeptide of the Fab fragment.

43: The bispecific antibody according to claim 42, wherein the first heavy chain region and/or second heavy chain region comprise a CH1 domain, and wherein the CH1 domain comprises the primary mutations and secondary mutations.

44-58. (canceled)

59: A composition comprising a bispecific antibody as defined in claim 1, and at least one pharmaceutically acceptable carrier.

60. (canceled)

61. (canceled)

62: A method of treating an individual in need thereof comprising administering to the individual a therapeutically-effective amount of the bispecific antibody of the invention polypeptide as defined in claim 1.

63: A method of treating or preventing cancer in an individual in need thereof comprising administering to the individual a therapeutically-effective amount of the bispecific antibody as defined in claim 1.

64-66. (canceled)

67: The bispecific antibody of claim 1, wherein the primary mutations comprise mutations at positions T/S114 in the CKappa or CLambda (according to EU or Kabat numbering) and T187 in the CH1 domain (according to EU numbering) of the immunoglobulin molecule or appended Fab and wherein said secondary mutations comprise mutations at positions T109 in the CKappa or CLambda (according to EU or Kabat numbering) and S183 in the CH1 domain (according to EU numbering) of the immunoglobulin molecule or appended Fab.

68: The bispecific antibody of claim 1, wherein the secondary mutations are T109P in the CKappa or CLambda (according to EU or Kabat numbering) and S183T in the CH1 domain (according to EU numbering) of the immunoglobulin molecule or appended Fab.

69: The bispecific antibody of claim 1, comprising the following primary mutations:
wherein the VL region of the immunoglobulin molecule comprises a mutation at position Q44E (according to IMGT numbering),
wherein the VH region of the immunoglobulin molecule comprises a mutation at position Q44R (according to IMGT numbering),
wherein the VL region of the Fab fragment comprises a mutation at position Q44R (according to IMGT numbering),
wherein the VH region of the Fab fragment comprises a mutation at position Q44E (according to IMGT numbering),
wherein the CH1 region of the immunoglobulin molecule comprises a mutation at position H168A and at position F170G (according to EU numbering), and
wherein the CKappa or CLambda region of the immunoglobulin molecule comprises a mutation at position L135Y and at position S176W (according to Kabat numbering),
wherein the CH1 region of the Fab fragment comprises a mutation at position T187E (according to EU numbering), and
wherein the CKappa or CLambda region of the Fab fragment comprises a mutation at position S114A and at position N137K (according to Kabat numbering),
and comprising the following secondary mutations:
wherein the CKappa or CLambda region of the appended Fab fragment comprises a T109P mutation (according to EU or Kabat numbering), and
wherein the CH1 region of the appended Fab fragment comprises an S183T mutation (according to EU numbering).
